(19) United States
(12) Patent Application Publication
(10) Pub. No.: US 2024/0342723 A1
(43) Pub. Date: Oct. 17, 2024

(54) AGRICULTURAL SAMPLE HANDLING SYSTEM AND RELATED METHODS

(71) Applicant: Precision Planting LLC, Tremont, IL (US)

(72) Inventors: Nicholas Minarich, Shorewood, IL (US); Todd Swanson, Morton, IL (US); Dale M. Koch, Tremont, IL (US)

(21) Appl. No.: 18/682,535

(22) PCT Filed: Sep. 7, 2022
(86) PCT No.: PCT/IB2022/058414

§ 371 Date: Feb. 9, 2024

Related U.S. Application Data
(60) Provisional application No. 63/245,278, filed on Sep. 17, 2021.
(60) Provisional application No. 63/264,059, filed on Nov. 15, 2021.
(60) Provisional application No. 63/264,062, filed on Nov. 15, 2021.
(60) Provisional application No. 63/264,065, filed on Nov. 15, 2021.
(60) Provisional application No. 63/370,072, filed on Aug. 1, 2022.
(60) Provisional application No. 63/370,077, filed on Aug. 1, 2022.
(60) Provisional application No. 63/370,081, filed on Aug. 1, 2022.

(51) Int. Cl. B01L 3/00

(52) U.S. Cl. CPC B01L 3/50825; B01L 2300/042; B01L 2300/045; B01L 2400/0478

(57) ABSTRACT

A sample container for an agricultural sample such as soil comprises: an elongated tubular body defining a longitudinal axis, a top end, a bottom end, and an internal cavity extending between the ends configured for holding the sample; a first cap detachably coupled to the top end; and a second cap slideably disposed in the cavity, the second cap being movable in opposing directions between the top and bottom ends.

### CROSS-REFERENCE TO RELATED APPLICATIONS

The present application claims priority to U.S. Provisional Patent Application No. 63/245,278 filed 17 Sep. 2021; U.S. Provisional Patent Application No. 63/264,059 filed 15 Nov. 2021; U.S. Provisional Patent Application No. 63/264,062 filed 15 Nov. 2021; U.S. Provisional Patent Application No. 63/264,065 filed 15 Nov. 2021; U.S. Provisional Patent Application No. 63/370,072 filed 1 Aug. 2022: U.S. Provisional Patent Application No. 63/370,077 filed 1 Aug. 2022; and U.S. Provisional Patent Application No. 63/370,081 filed 1 Aug. 2022. The foregoing applications are incorporated herein by reference in their entireties.

### BACKGROUND

The present disclosure relates generally to agricultural sampling and analysis, and more particularly to a system for packaging, tracking, and handling an agricultural sample such as without limitation soil for chemical analysis.

Periodic soil testing is an important aspect of the agricultural arts. Test results provide valuable information on the chemical makeup of the soil such as plant-available nutrients and other important properties (e.g., levels of nitrogen, magnesium, phosphorous, potassium, pH, etc.) so that various amendments may be added to the soil to maximize the quality and quantity of crop production.

In some existing soil sampling processes, collected bulk agricultural samples such as soil or other agricultural materials may require some form of packaging to facilitate transport and further preparation and processing for eventual chemical analysis. The packaging further protects the integrity of the samples until processed. In addition, a means for tracking where samples were collected from in the agricultural field is necessary to associate the chemical analysis results with a particular portion of the field. Furthermore, a system for unloading the packaged sample is necessary to allow further processing and analysis of the sample.

Improvements in agricultural sample handling are desired.

### BRIEF SUMMARY

The present disclosure provides automated programmable processor-controlled agricultural sample packaging and handling systems, and related methods for containerizing an agricultural sample and then unloading a sample container. In some embodiments, the container may be a cylindrical sample tube capped at or near both ends. The sample may be a soil sample in some non-limiting embodiments, or other agricultural-related materials described further herein.

The packaging system may comprise a packaging apparatus which receives bulk soil sample material collected by an automated sample collection device/probe or manually, extracts portions of the material, and transfers the extracted portions to the sample container which may then be capped. The sample extraction and containerization process may be automatically controlled by a programmable system controller which communicates with multiple sensors which monitor the operation and position of the various components of the packaging equipment to control its operation. The system may include sample tracking comprising assigning a unique tracking ID to each sample which can be correlated to the location in the agricultural field or elsewhere where the sample was obtained. A machine-readable tracking tag may be affixed to the sample tube or its end caps for that purpose. In one embodiment, RFID (radio frequency identification) may be used. The tracking tag may therefore be an RFID tag readable by an RFID reader. In other embodiments, the tracking tag may be a bar code readable by a visual barcode scanner. Other forms of readable tracking tags and related systems may be used.

A sample container unloading system may be comprise a staging rack and sample container unloading apparatus operably coupled together. The staging rack is configured to receive and hold a plurality of the filled sample containers from the packaging system. A transfer mechanism may transfer and load the sample tubes from the rack sequentially into the unloading apparatus which operates to uncap the tubes and eject the sample contents for further processing. The sample tube staging, transfer mechanism, and sample unloading operation may be fully automated and controlled by the programmable controller.

In one aspect, an agricultural sample unloading system comprises: a sample staging rack comprising at least one inclined feed ramp configured for receiving an elongated sample tube configured for holding the sample, the sample tube including a first end cap and a second end cap; an unloading apparatus coupled to the staging rack, the unloading apparatus configured to receive the sample tube from the staging rack; and a transfer mechanism operable to transfer the sample tube from the staging rack to the unloading apparatus. The unloading apparatus comprises a rotatable carriage configured to hold and rotate the sample tube in opposing directions during the sample unloading process. The unloading apparatus further comprises a loading mechanism operable to load the sample tube into the carriage.

In another aspect, a method for unloading an agricultural sample container comprises: inserting a capped sample tube containing a sample into an unloading apparatus; rotating the sample tube a first time to an upright vertical position; uncapping the sample tube which creates an open top end; rotating the sample tube a second time to an inverted vertical position; and ejecting the sample from the sample tube. The unloading apparatus comprises a movable carriage including an elongated receptacle into which the sample tube is inserted and which performs the foregoing movements of the sample tube.

In another aspect, an agricultural sample container comprises: an elongated tubular body defining a longitudinal axis, a top end, a bottom end, and an internal cavity extending between the ends configured for holding the sample; a first cap detachably coupled to the top end; and a second cap slideably disposed in the cavity, the second cap being movable in opposing directions between the first and second ends. The second cap comprises a base and a plurality of longitudinally-extending retention protrusions extending downwards from the base. The body in some embodiments further comprises a plurality of circumferentially spaced apart retention slots configured to lockingly engage the retention protrusions.

Although the agricultural sample packaging system may be described herein with reference to containerizing soil samples which represents only a single category of use for the disclosed embodiments, it is to be understood that the same packaging systems including the apparatuses and related processes may further be used for processing other types of agricultural related samples including without limitation vegetation/plant, forage, manure, feed, milk, or other types of samples. The disclosure herein should therefore be considered broadly as an agricultural sample packaging system amenable for extracting and containerizing many different types of samples from bulk “as collected” sample material regardless of the method for collection. Accordingly, the present agricultural sample packaging system disclosed is expressly not limited to use of packaging soil samples alone for chemical analysis of properties of interest.

### BRIEF DESCRIPTION OF THE DRAWINGS

The present disclosure will become more fully understood from the detailed description and the accompanying drawings, wherein like elements are labeled similarly and in which:

FIG. 1 is a high-level process flow chart providing a summary overview of the agricultural sample packaging process according to the present disclosure;

FIG. 2 is a schematic system diagram of a programmable processor-based central processing unit (CPU) or system controller for controlling the systems and apparatuses disclosed herein associated with the agricultural sample packaging system;

FIG. 3 is right top front perspective view of the sample packaging apparatus of the system;

FIG. 4 is an exploded perspective view thereof showing the outer protective housing removed;

FIG. 5 is a bottom left rear perspective of the packaging apparatus;

FIG. 6 is an exploded perspective view thereof;

FIG. 7 is a top view of the packaging apparatus;

FIG. 8 is a bottom view thereof;

FIG. 9 is a front view thereof without the housing;

FIG. 10 is a rear view thereof;

FIG. 11 is a right side view thereof;

FIG. 12 is a left side view thereof;

FIG. 13 is a top right front perspective view thereof;

FIG. 14 is a top left rear perspective view thereof;

FIG. 15 is a front view thereof with some equipment removed to better show the sample blade mechanism components;

FIG. 16 is a top view thereof;

FIG. 17 is a right top front perspective view thereof;

FIG. 18 is a left top front perspective view thereof;

FIG. 19 is a left bottom rear perspective view with the lower portion of the apparatus support frame removed;

FIG. 20 is a top perspective view of a sample blade;

FIG. 21 is a top view of the sample blade showing a spring pack associated with the blade;

FIG. 22 is a front view of the sample packaging apparatus showing one operating position of the mechanism with sample funnel attached and the rotatable carousel with sample container supported thereby in the outward open position;

FIG. 23 is a front cross-sectional view thereof;

FIG. 24 is a side cross-sectional view of the apparatus showing the funnel door of the funnel in the closed position;

FIG. 25 is a front cross-sectional view of the apparatus showing the carousel and sample container rotated to the inward closed position ready for filling with the agricultural sample material;

FIG. 26 is bottom view of the apparatus showing the carousel in the outward open position;

FIG. 27 is a bottom view of the apparatus showing the carousel in the inward closed position;

FIG. 28 is a front cross-sectional view of the apparatus showing the sample and cleaning blades of the apparatus in their retracted positions;

FIG. 29 is a side cross-sectional view of the apparatus showing the funnel door in the open position to admit sample material into the bulk material chamber as indicated by the directional arrow;

FIG. 30 is a front cross-sectional view of the apparatus showing the sample blades in their projected positions inserted through the bulk material chamber and die block;

FIG. 31 is a front cross-sectional view thereof showing the sample transfer piston-plunger in its downward extended/projected position;

FIG. 32 is a front cross-sectional view thereof showing the sample transfer piston-plunger returned to its upward retracted position;

FIG. 33 is front cross-sectional view thereof showing the cleaning blades in their projected position inserted through the sample collection chamber and die block;

FIG. 34 is a front cross-sectional view thereof showing the cleaning blades returned to their retracted positions;

FIG. 35 is a front cross-sectional view thereof showing the compaction transfer piston-plunger in its downward extended/projected position;

FIG. 36 is a front cross-sectional view thereof showing the compaction piston-plunger returned to its upward retracted position;

FIG. 37 is partial front perspective view of the carousel showing a collapsible embodiment of the container holder with container sensor;

FIG. 38 is a bottom view of the apparatus showing the carousel and sample container rotated to the inward closed position ready for filling with the agricultural sample material;

FIG. 39 is bottom view of thereof showing the carousel in the outward open position to enable removal of a filled sample container from the apparatus;

FIG. 40 is a front cross-sectional view of the apparatus showing the sample transfer piston-plunger and compaction plunger in their downward projected positions;

FIG. 41 is an enlarged partial front cross-sectional view of the chambers and die block of the apparatus;

FIG. 42 is a first bottom perspective view thereof;

FIG. 43 is a second bottom perspective view thereof;

FIG. 44 is an enlarged bottom perspective view of the carousel of the apparatus;

FIG. 45 is a bottom exploded perspective view of the sample container;

FIG. 46 is a side view of the collapsible embodiment of the cup hold of the carousel showing the push cap actuator;

FIG. 47 is a top perspective view of the sample container;

FIG. 48 is an exploded perspective view thereof;

FIG. 49 is a bottom exploded perspective view thereof;

FIG. 50 is a bottom perspective view of the slideable spring-action push cap of the sample container;

FIG. 51 is a side view of the sample container;

FIG. 52 is a side cross sectional view thereof;

FIG. 53 is a top view thereof;

FIG. 54 is a bottom view thereof;

FIG. 55 is an enlarged detail taken from FIG. 52 showing the snap-fit top cap and corresponding snap-fit features of the sample container and cap;

FIG. 56 is a first perspective view of an agricultural sample unloading system according to the present disclosure, including a sample container staging rack, unloading apparatus, and container transfer mechanism;

FIG. 57 is an enlarged detail taken from FIG. 56;

FIG. 58 is a side view of the sample unloading system;

FIG. 59 is a distal end view thereof;

FIG. 60 is a top view thereof;

FIG. 61 is an enlarged detail taken from FIG. 60;

FIG. 62 is a first side view of the container staging rack alone;

FIG. 63 is a distal end view thereof;

FIG. 64 is a second side view thereof showing the side opposite the side in FIG. 62;

FIG. 65 is a proximal end view of the staging rack;

FIG. 66 is a longitudinal cross sectional view of the staging rack taken from FIG. 63;

FIG. 67 is a transverse view thereof taken from FIG. 66;

FIG. 68 is an enlarged detail taken from FIG. 67;

FIG. 69 is a first side perspective view of the staging rack with the side panel plate removed to reveal the inclined feed ramps of the rack;

FIG. 70 is a first enlarged detail taken from FIG. 69;

FIG. 71 is a second enlarged detail taken from FIG. 69;

FIG. 72 is a second side perspective view of the staging rack opposite the first side perspective view of FIG. 69 with the side panel plate removed to reveal the inclined feed ramps of the rack;

FIG. 73 is an enlarged detail taken from FIG. 72;

FIG. 74A is a partial perspective view of the unloading apparatus showing the container closure plate rotated outwards from the container positioned in the rotatable carriage of the apparatus;

FIG. 74B is a partial perspective view thereof but showing the closure plate rotated inwards to cover the open top end of the container;

FIG. 74C is a partial perspective view thereof showing the carriage rotated 90 degrees from FIGS. 74A-B and the closure plate rotated outwards;

FIG. 75 is a top perspective view of the entire unloading apparatus;

FIG. 76 is a bottom perspective view thereof;

FIG. 77 is a front view thereof;

FIG. 78 is an enlarged detail taken from FIG. 77;

FIG. 79 is a rear view of the unloading apparatus;

FIG. 80 is a left end view thereof;

FIG. 81 is a right end view thereof;

FIG. 82 is top view thereof;

FIG. 83 is a bottom view thereof;

FIG. 84 is a front view of the container unloading apparatus showing the container carriage and container rotated into a horizontal position/orientation;

FIG. 85 is a cross-sectional view of FIG. 84;

FIG. 86 is a front view of the unloading apparatus showing the carriage and container rotated into a vertical upright position/orientation and decapper engaged with the top cap of the container;

FIG. 87 is a cross-sectional view of FIG. 86;

FIG. 88 is a front view of the unloading apparatus showing the carriage shifted downward to remove the top cap of the container and decapper having moved away from the container with the top cap;

FIG. 89 is a front view thereof showing a closure plate moved onto and closing the top of the open container;

FIG. 90 is a front view of the unloading apparatus showing the carriage and container partially rotated from the upright position/orientation;

FIG. 91 is a front view of the unloading apparatus showing the carriage and container rotated into a vertical inverted position/orientation;

FIG. 92 is a front view of the unloading apparatus showing the carriage shifted downward towards an unloading port of the apparatus;

FIG. 93 is a front view thereof showing the sample ejector piston-plunger moved downwards to eject the sample for the container;

FIG. 94 is a cross sectional view of FIG. 93;

FIG. 95 is a first cross sectional view of the unloading apparatus in a series of sequential views illustrating a sample container unloading process according to the present disclosure;

FIG. 96 is a second cross sectional view thereof;

FIG. 97 is a third cross sectional view thereof;

FIG. 98 is a fourth cross sectional view thereof;

FIG. 99 is a fifth cross sectional view thereof;

FIG. 100 is a sixth cross sectional view thereof;

FIG. 101 is a seventh cross sectional view thereof;

FIG. 102 is an eighth cross sectional view thereof;

FIG. 103 is a ninth cross sectional view thereof;

FIG. 104 is a tenth cross sectional view thereof;

FIG. 105 is a eleventh cross sectional view thereof;

FIG. 106 is a twelfth cross sectional view thereof;

FIG. 107 is a thirteenth cross sectional view thereof;

FIG. 108 is a fourteenth cross sectional view thereof;

FIG. 109 is a high level flow chart showing the general steps of the sample container unloading process;

FIG. 110 is a perspective view of the decapper of the unloading apparatus;

FIG. 111 is a perspective view of a sample container gripping mechanism according to the present disclosure;

FIG. 112 is a plan view thereof showing the gripping mechanism in an outward open position disengaged from the sample container; and

FIG. 113 is a plan view thereof showing the gripping mechanism in an inward closed position lockingly engaged with the sample container.

All drawings are not necessarily to scale. Components numbered and appearing in one figure but appearing un-numbered in other figures are the same components unless expressly noted otherwise. Any reference herein to a figure by a whole figure number which may appear in multiple figures bearing the same whole number but with different alphabetical suffixes shall be constructed as a general reference to all of those figures unless expressly noted otherwise.

### DETAILED DESCRIPTION

The features and benefits of the present disclosure are illustrated and described herein by reference to exemplary (“example”) embodiments. This description of exemplary embodiments is intended to be read in connection with the accompanying drawings, which are to be considered part of the entire written description. Accordingly, the disclosure expressly should not be limited to such exemplary embodiments illustrating some possible non-limiting combination of features that may exist alone or in other combinations of features.

In the description of embodiments disclosed herein, any reference to direction or orientation is merely intended for convenience of description and is not intended in any way to limit the scope of the present disclosure. Relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise.

As used throughout, any ranges disclosed herein are used as shorthand for describing each and every value that is within the range. Any value within the range can be selected as the terminus of the range. In addition, all references cited herein to prior patents or patent applications are hereby incorporated by reference in their entireties. In the event of a conflict in a definition in the present disclosure and that of a cited reference, the present disclosure controls.

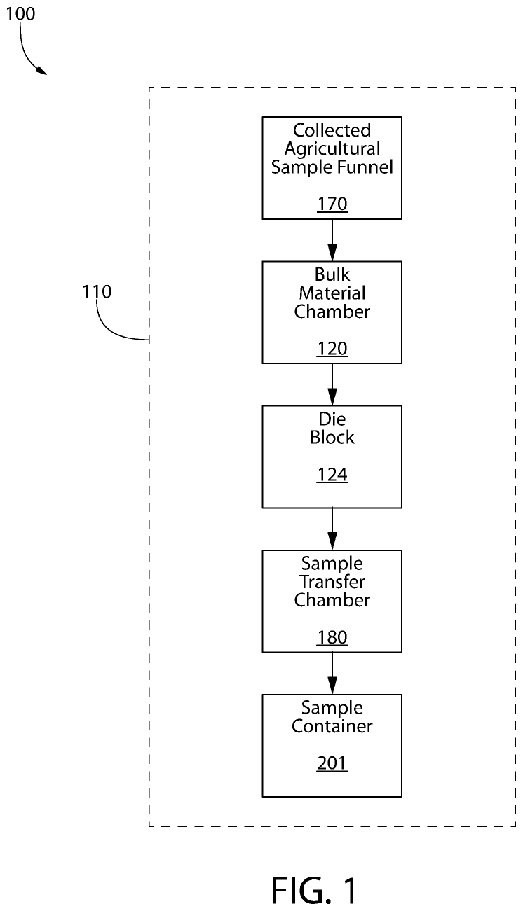

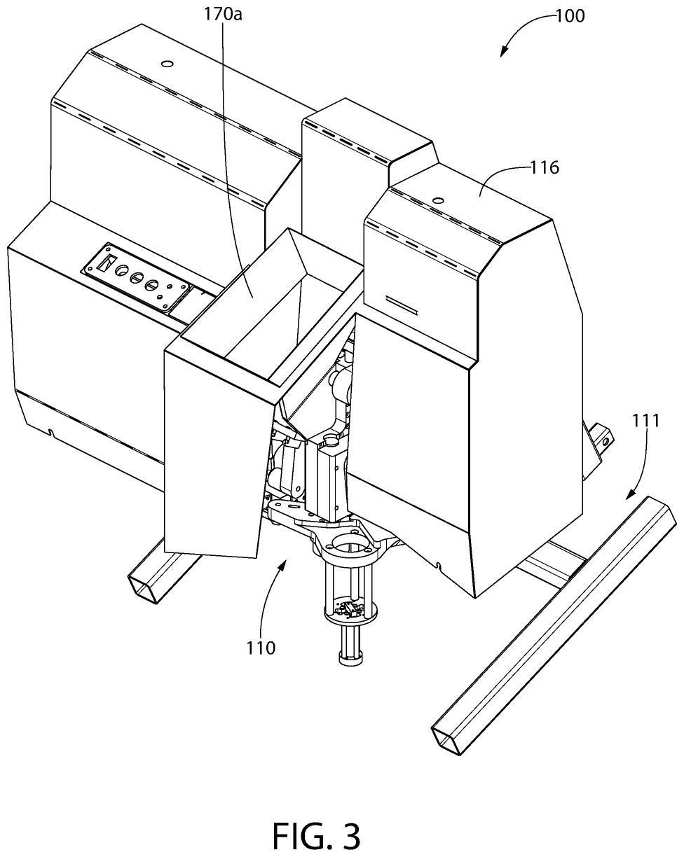

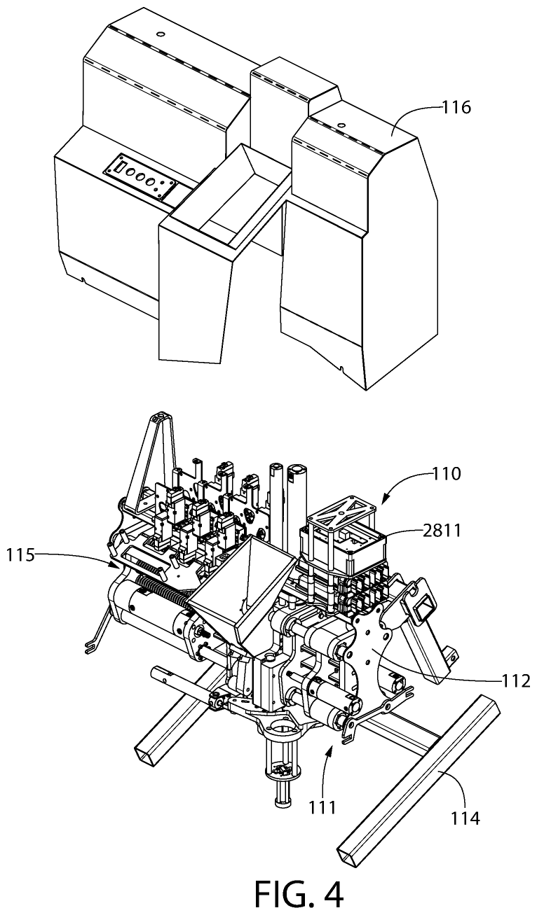

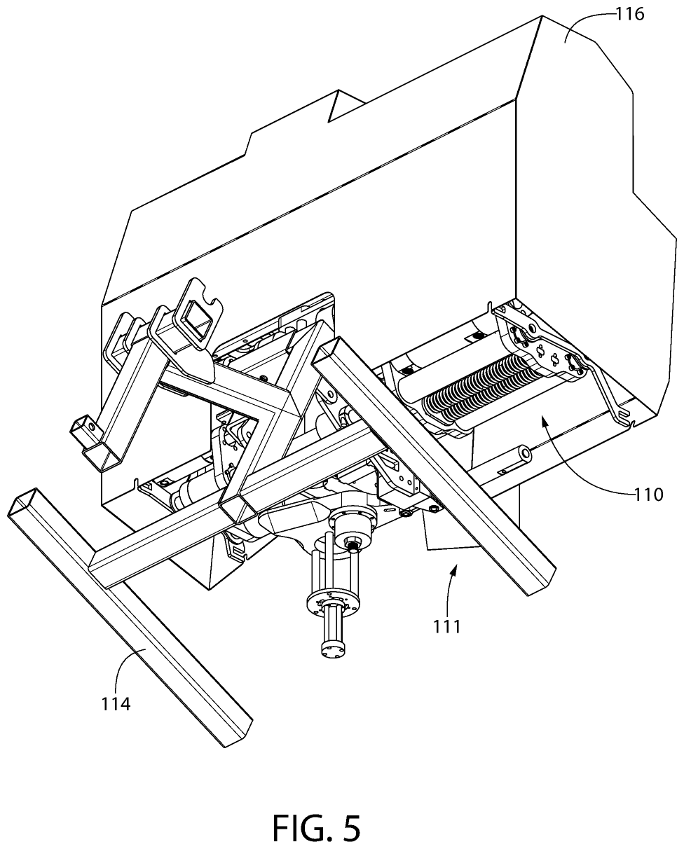

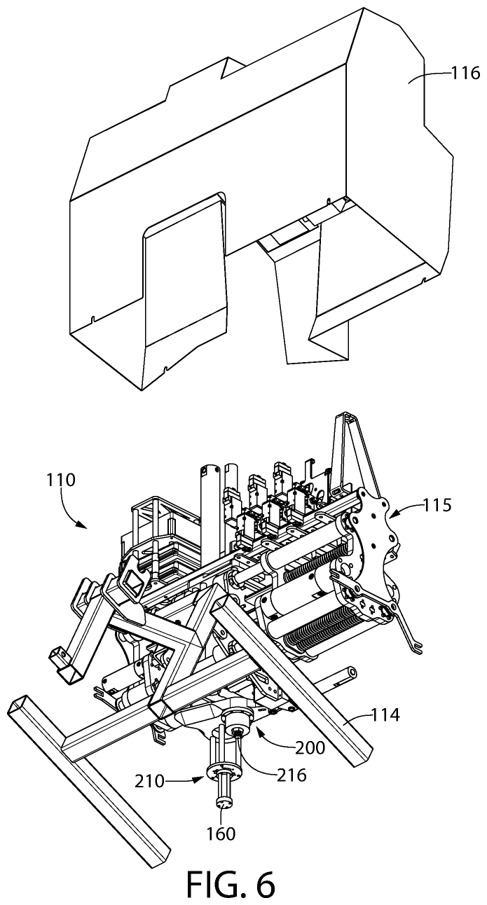

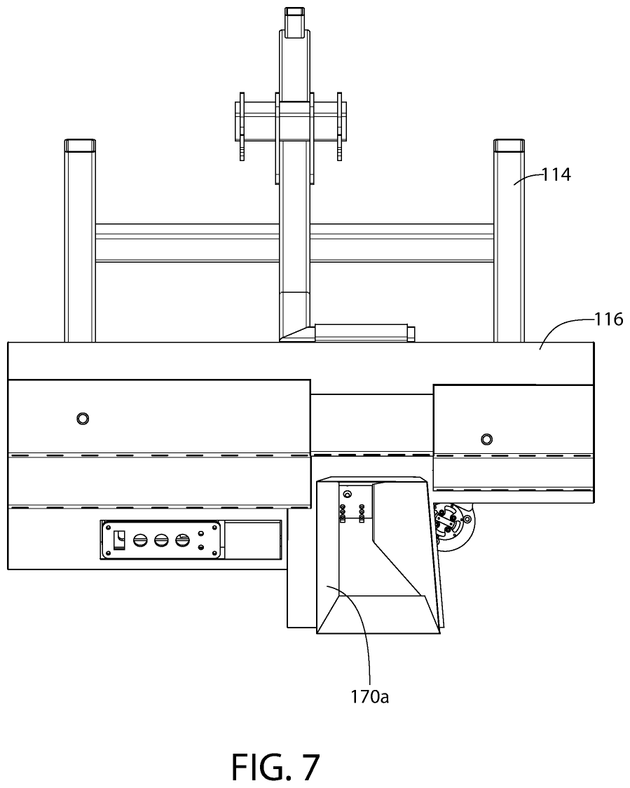

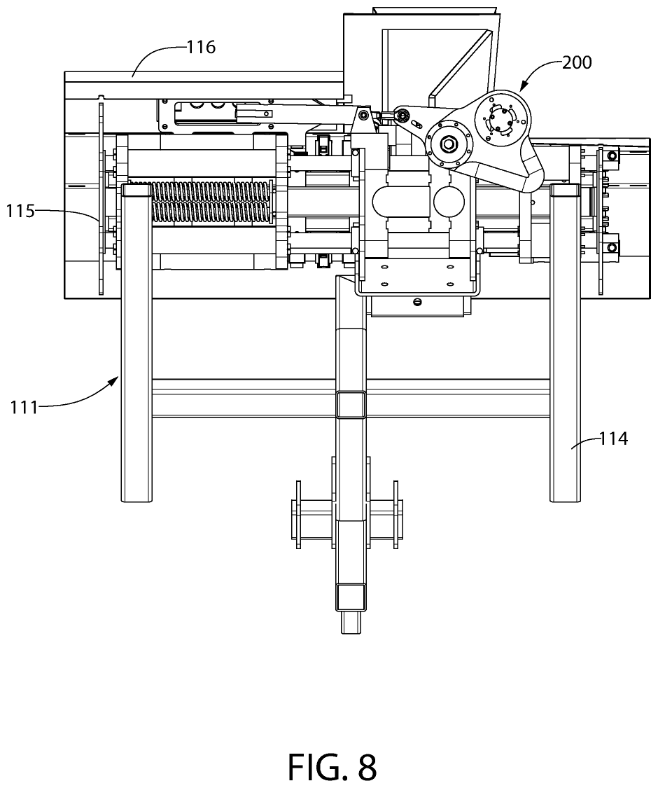

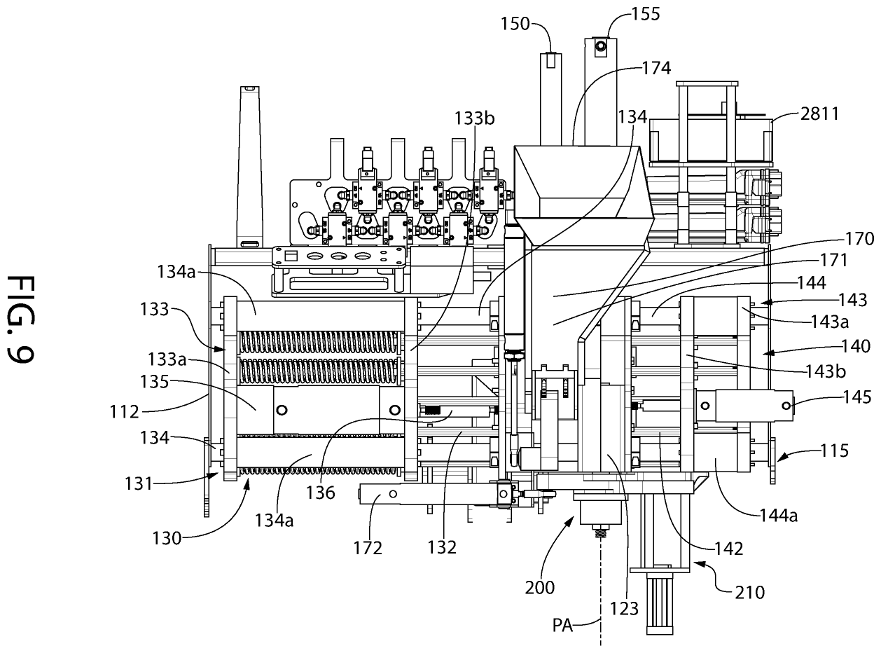

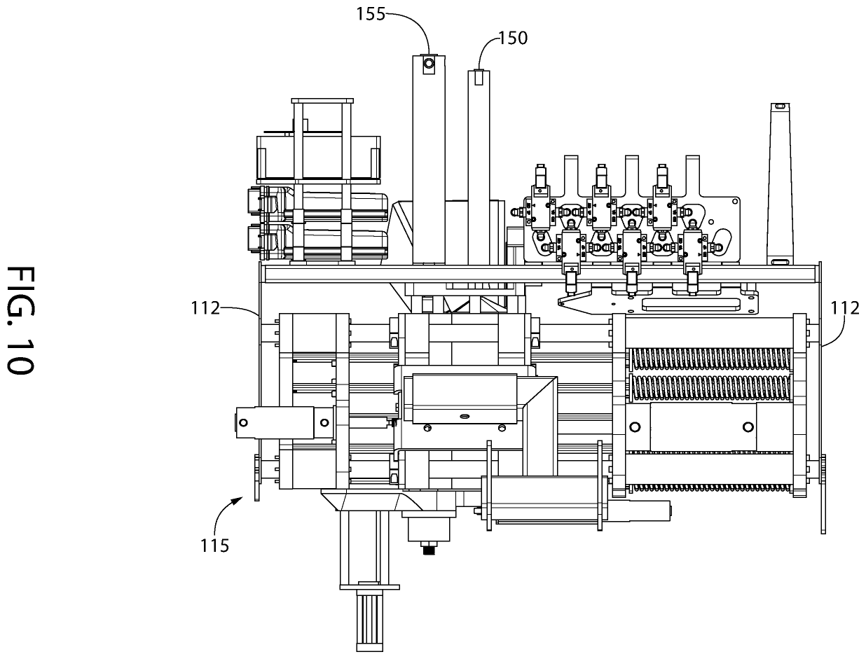

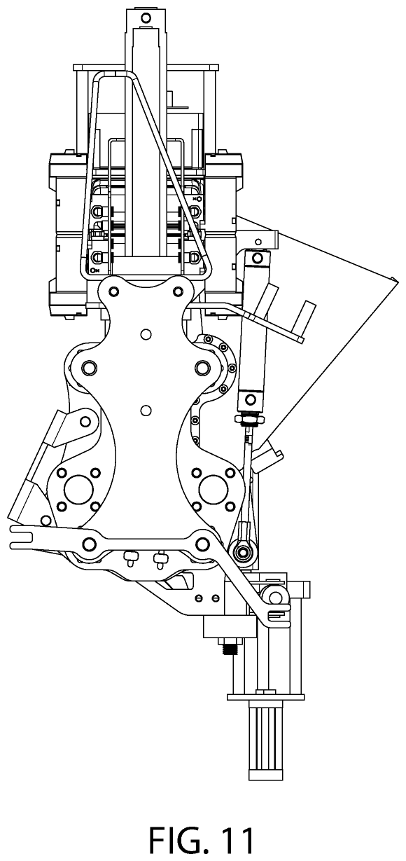

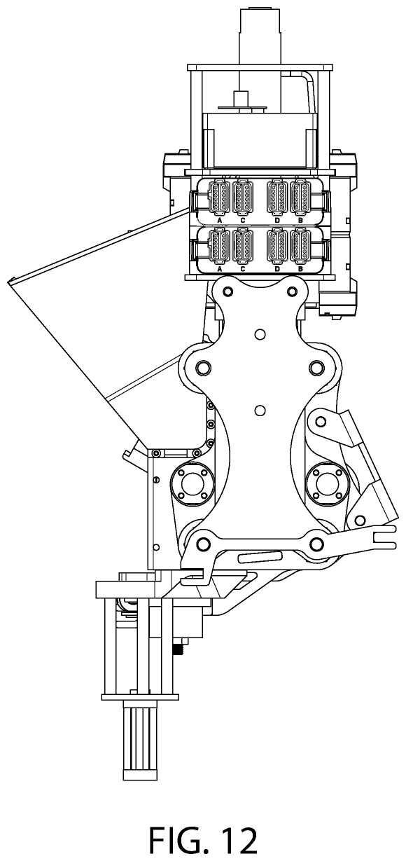

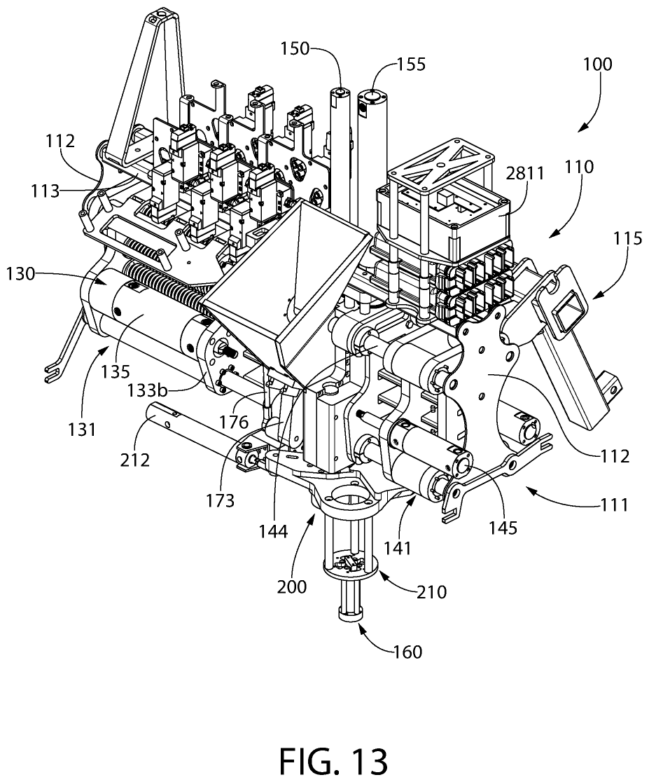

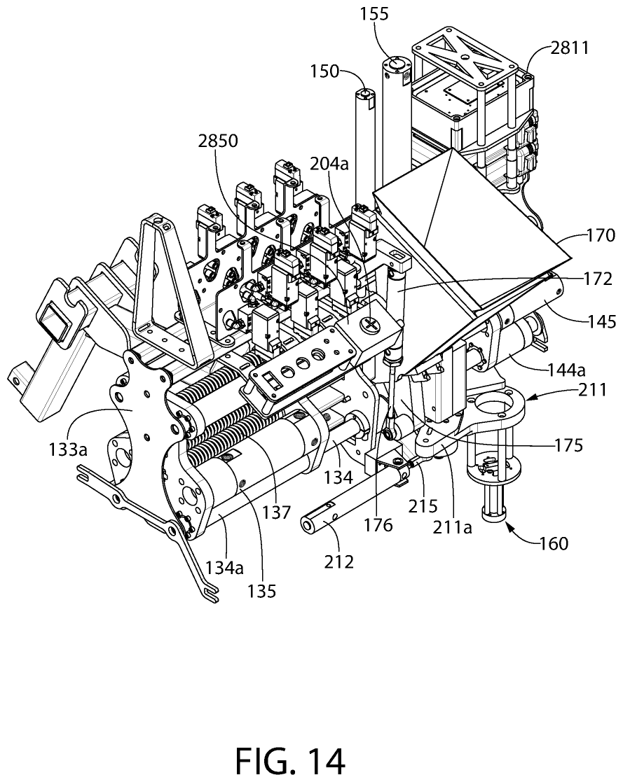

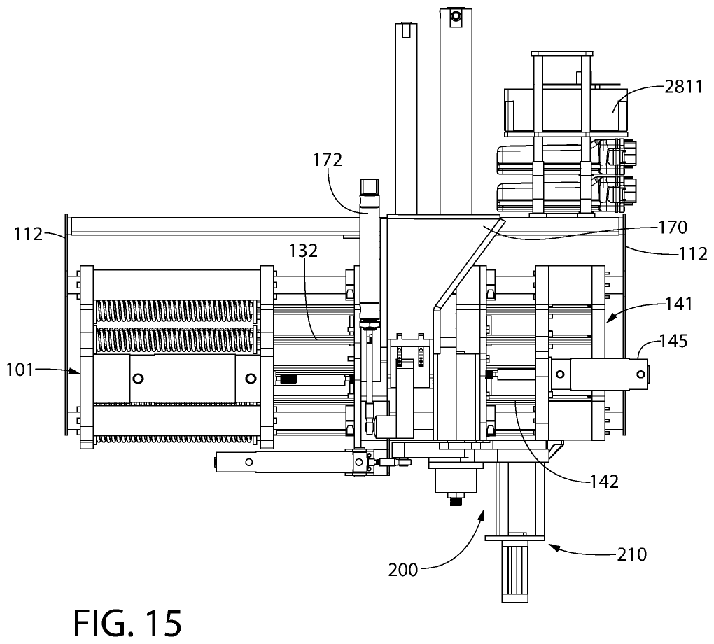

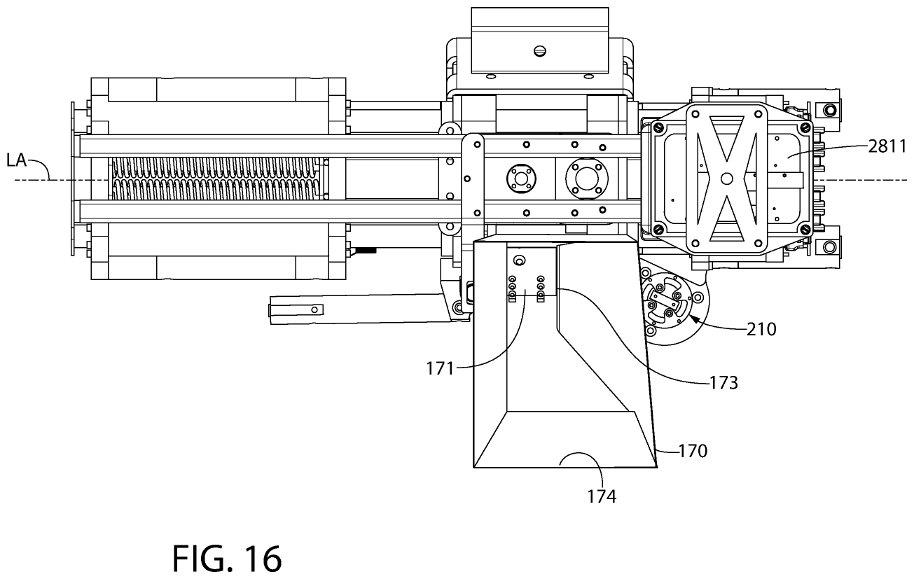

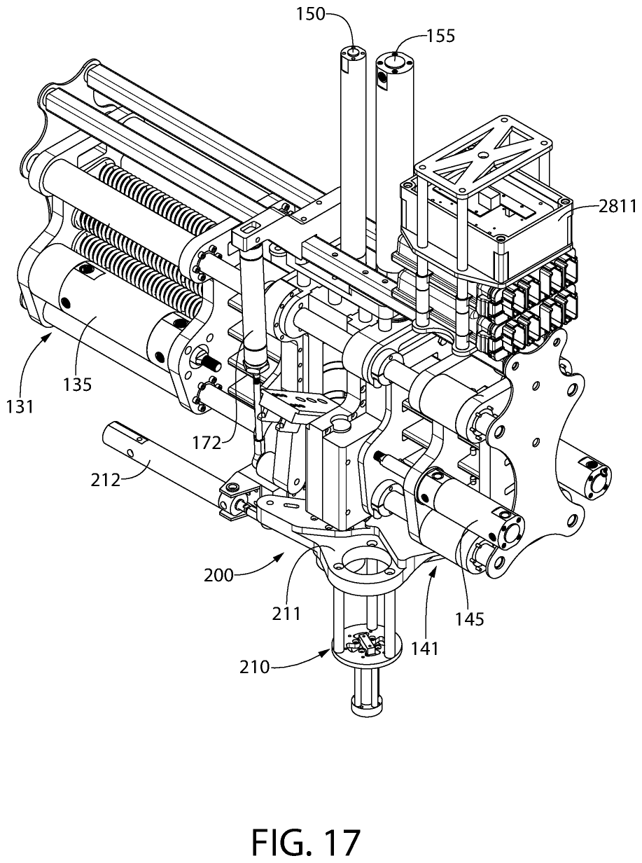

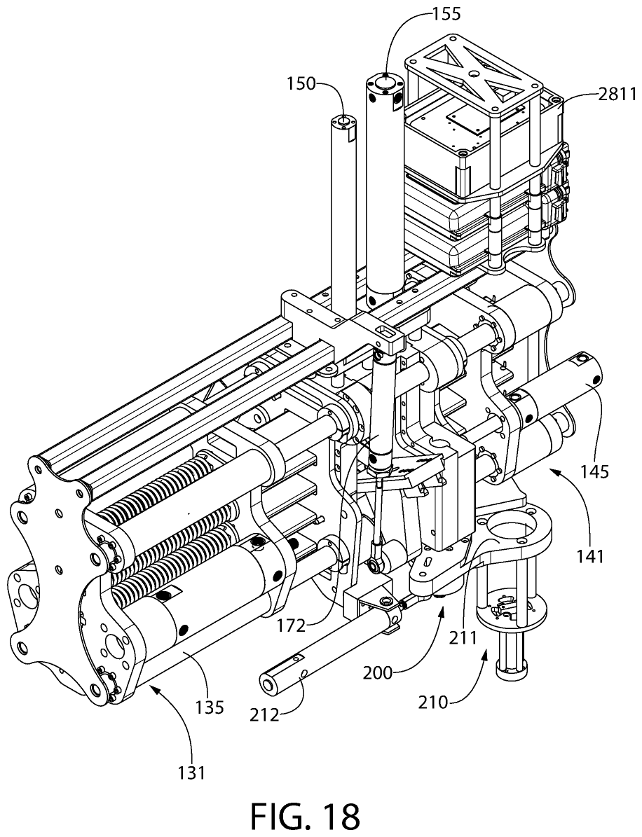

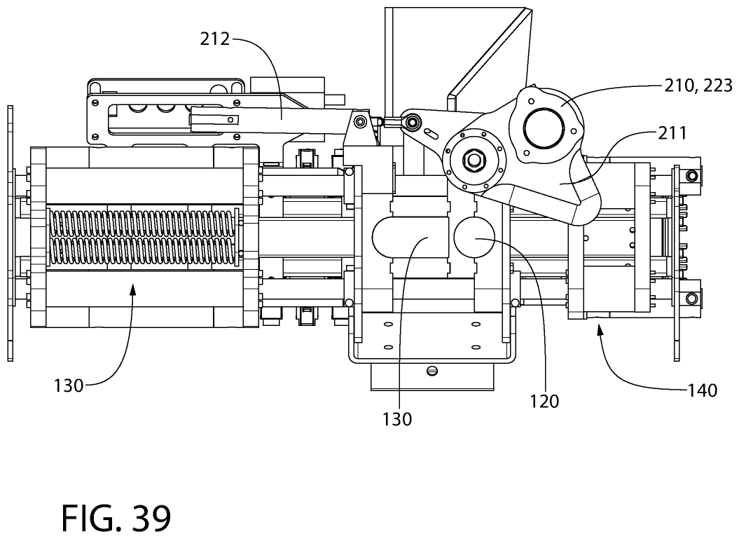

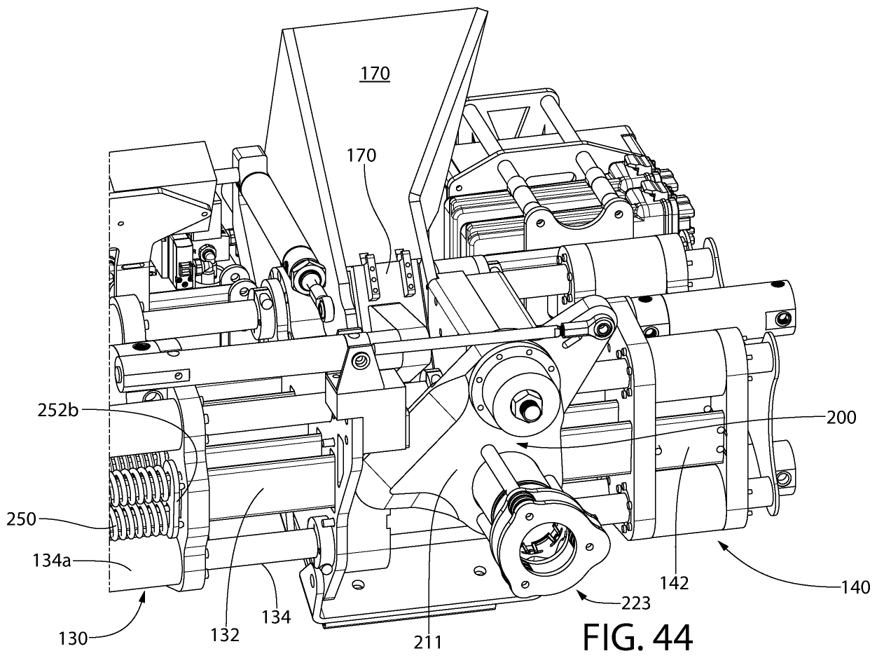

FIGS. 1-46 show one embodiment of an agricultural sample packaging system 100 and various components thereof according to the present disclosure. The packaging system may be used for and will be described for convenience with containerizing soil samples as an illustrative but not limiting use.

Packaging system 100 generally comprises agricultural sample packaging apparatus 110 defining a front 110a, rear 110b, right lateral side 110c, left lateral side 110d, top 110e, and bottom 110f for convenience of reference and not limitation. Apparatus 110 can be horizontally and laterally elongated in structure and defines a longitudinal axis LA extending horizontally through the right and left lateral sides and intersects the geometric centerline of the apparatus. An outer protective housing 116 of suitable configuration and material may be provided to protect a majority of the components of the packaging apparatus 110 including electronic components from dust, debris, direct rainfall, impacts, etc.

Packaging apparatus 110 includes a support frame 111 including an upper sub-frame 115 comprising a pair of laterally spaced apart vertical end supports 112 and one or more cross supports 113 spanning between and fixedly coupled to the end support members to form a self-supporting rigid frame structure. A lower base sub-frame 114 of the chassis 111 may be provided in some embodiments which is coupled to the upper sub-frame 115 and elevates and/or spaces the upper sub-frame above a support surface or object. Base sub-frame 114 may comprise an assemblage or weldment of plural structural members (e.g., tubes, rods, L-angles, I-beams, C-beams, etc.) configured to conform to and the mount the apparatus to a support surface or object. The support surface or object may be a stationary article or surface, or movable such as part of a self-powered or pulled wheeled vehicle (e.g., ATV, truck, trailer, etc.). Accordingly, numerous configurations of base sub-frames are possible depending on the mounting needs.

Upper sub-frame 115 of support frame 111 provides a mounting platform for and supports the functional and movable electronic and non-electronic components of the packaging apparatus 110. In one embodiment, the apparatus generally comprises a bulk material chamber 120, a sample transfer or collection chamber 180, a sample blade mechanism 130, a cleaning blade mechanism 140, a compaction piston-plunger 150, sample transfer piston-plunger 155, an optional container end cap actuator 160, funnel 170 with associated funnel door 171 and door actuator 172, and a rotatable container carousel 200 configured to removably hold the sample container 201 for the soil sample filling operation.

In the non-limiting illustrated embodiment, agricultural sample container 201 may be a hollow cylindrical sample tube 202 having a construction and customized features adapted for use with packaging apparatus 110 and unloading system 300 described below to containerize agricultural samples such as soil samples or others, and unload the containerized samples. Accordingly, sample tube 202 is distinguishable from ordinary tubes which may have capped ends.

Sample tube 202 has an elongated cylindrical tubular and hollow body defining cylindrical wall 202a terminated by a top end 203a and bottom end 203b closed and sealed by a pair of circular end caps 204. An internal cavity 207 extends between the ends along a longitudinal axis LA2 of the tube and holds the agricultural sample material. Caps 204 may be made of metallic or non-metallic (e.g., plastic or other) materials. In one embodiment, the tube body and caps 204 are formed of plastic. One end cap 204a may be a fixed or stationary cap configured for detachable coupling to top end 203a of the container 201. The other remaining end cap may be a movable plunger-action “push” cap 204b which is slideably received inside the tube 202 adjacent to bottom end 203b of the tube. Push cap 204b is slideably moveable from bottom end 203b of the tube towards the other top end 203a and vice-versa during the tube fill and unloading operations. Bottom end 203b of tube 202 in some embodiments may include anti-rotation features to rotationally lock the tube in position when mounted to the packaging apparatus 110, as further described herein.

One unique aspect of sample tube 202 is push cap 204b which includes a disk-shaped circular base 208 and plurality of downwardly and outwardly projecting spring-action retention legs or protrusions 205 configured to slideably engage the interior walls of the sample tube 202. Retention protrusions 205 may be separately mounted to the perimeter and peripheral edge of base 208 of cap 204b, or may be integrally formed as part of a single monolithic unitary cap structure as illustrated herein. In one preferred but non-limiting embodiment, the push cap 204b including base 208 and retention protrusions 205 may be such a one-piece unitary molded structure made of a suitable semi-rigid but resiliently deformable plastic material having an elastic memory (e.g., polyethylene, polypropylene, etc.). Retention protrusions 205 in other embodiments, however, may be separate elements formed of spring metal or resilient deformable plastic elements affixed to cap 204b.

In one embodiment, retention protrusions 205 may each have a somewhat squared-off or U-shaped configuration as shown; however, other shaped retention protrusions may be used and the shape does not limit the invention. This gives the push cap 204b a somewhat castellated shape. The free terminal ends 205a of the retention protrusions may be outwardly flared forming outward protruding locking tabs 205c which can positively engage corresponding complementary configured arcuately curved and elongated retention slots 202c of sample tube 202. This gives the protrusions 205 a somewhat L-shaped configuration with the protrusions appearing as downwardly extending legs from base 208 of cap 204b with out-turned ends. Slots 202c are oriented cross-wise in the tubular sample tube body perpendicularly to the length of its cylindrical wall 202a. The protrusions 205 may be circumferentially spaced apart as shown around the entire perimeter and periphery of the push cap 204b. Six retention protrusions 205 may be provided in one non-limiting embodiment; however, fewer or more protrusions may be provided. Retention protrusions 205 and particularly locking tabs 205c further slideably engage the interior surfaces of tube wall 202a when end cap 204b slides up and down inside tube cavity 207, as further described herein.

The circumferentially elongated retention slots 202c formed in the cylindrical walls 202a of the sample tube 202 are selectively engageable with retention protrusions 205a to lock or unlock the push cap 204b from the sample tube depending on the position of the cap inside the tube. Cap 204b therefore is sized in diameter to be fully inserted inside the internal cavity 207 of the sample tube whereas cap 204a is sized larger for affixation to the top end of the tube. Slots 202c may be through slots in one embodiment extending completely through the walls of the tube. Retention slots 202c are disposed proximate to bottom end 203b of sample tube 202 and spaced slightly upwardly and apart from the end of the tube. The opposite end of the tube receives fixed/stationary cap 204a. When sample tube 202 is placed in the carousel 200, the end of the tube with the retention slots 202c is preferably located at bottom for engagement with container end cap actuator 160 in some embodiments as further described herein.

Sample tube 202 may be formed of plastic, metal, or other suitable materials. In one preferred but non-limiting embodiments, the tube is made of plastic (e.g., polyethylene, polypropylene, etc.). The elongated tube body may be opaque or clear; the latter one allowing the sample to be visually inspected. Although the tube 202 is disclosed as being cylindrical in shape, other shapes and forms of sample containers may be used in other possible embodiments.

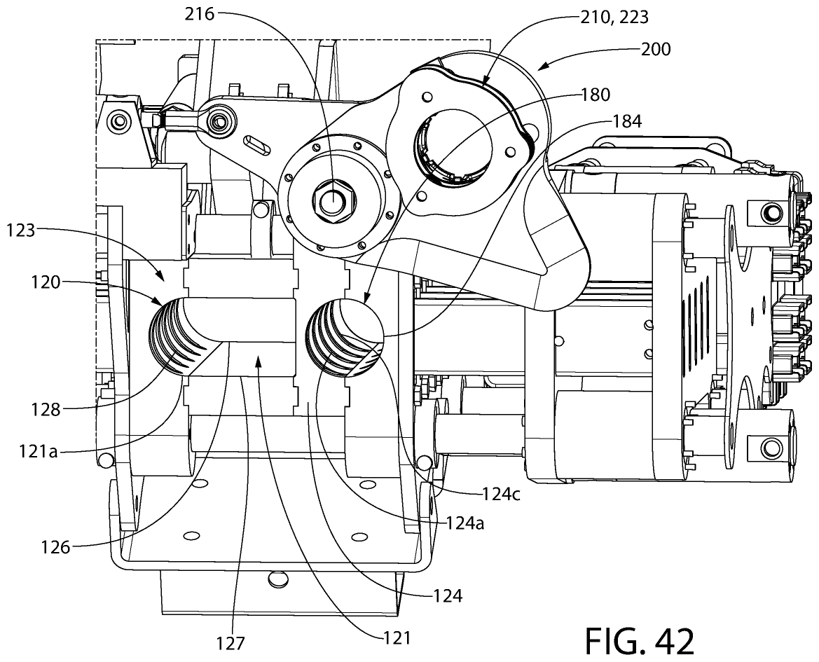

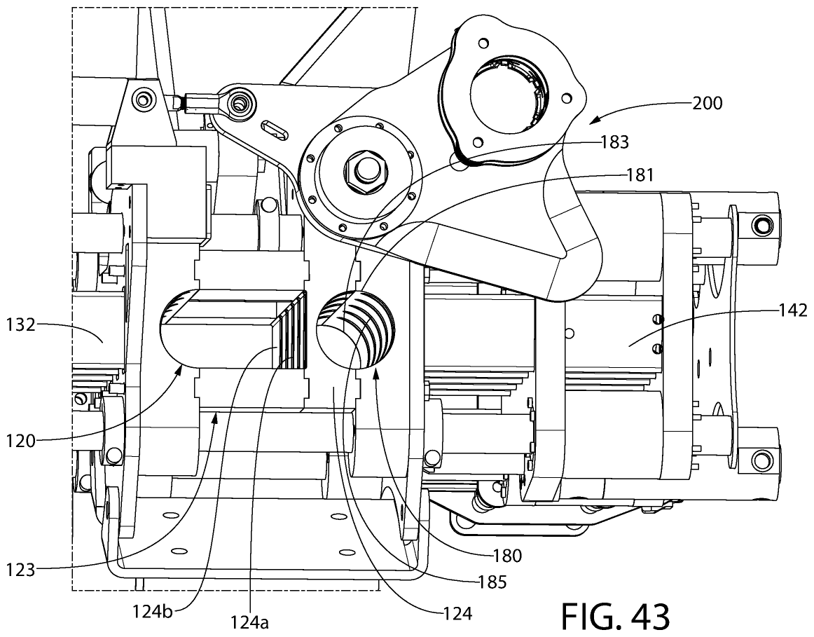

Bulk material chamber 120 and sample collection chamber 180 are each vertically-elongated hollow vessels located adjacent to each other and separated by an adjoining vertically-elongated die block 124 interspersed therebetween. Die block 124 forms a central division wall separating the chambers 120, 180 and comprises a set of vertically spaced apart die slots 124a extending completely therethrough and between the bulk material chamber and sample collection chamber. Slots 124a penetrate each of the chambers 120, 180 and are horizontally elongated in the front to rear direction. Die block 124 includes a flat wall side 124b which forms part of bulk material chamber 120 and opposite arcuately curved wall side 124c which forms part of sample collection chamber 180 as shown (see, e.g., FIGS. 42-43).

Chambers 120, 180 may be formed in separate chamber blocks 123 in one embodiment comprising blocks of solid material coupled to opposite sides of die block 124 (see, e.g., FIGS. 42-43. This construction allows the sample and cleaning through slots in the chamber blocks associated with the chambers 120 and 180 to be pre-fabricated prior to coupling to the die block. Chamber 180 may be partially formed in part of die block 124 such that the curved right wall side 124c of the die block forms part of the chamber 180 walls (see, e.g., FIGS. 42-43). Chamber block 123 and die block 124 are supported by upper sub-frame 115 between the ends of the packaging apparatus 110 as shown.

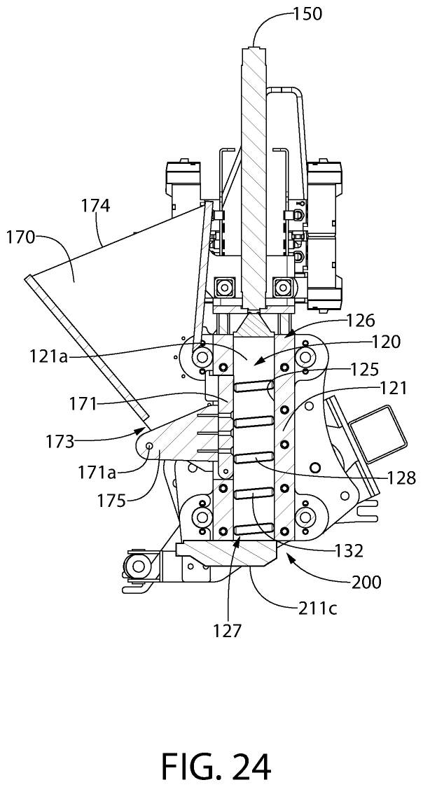

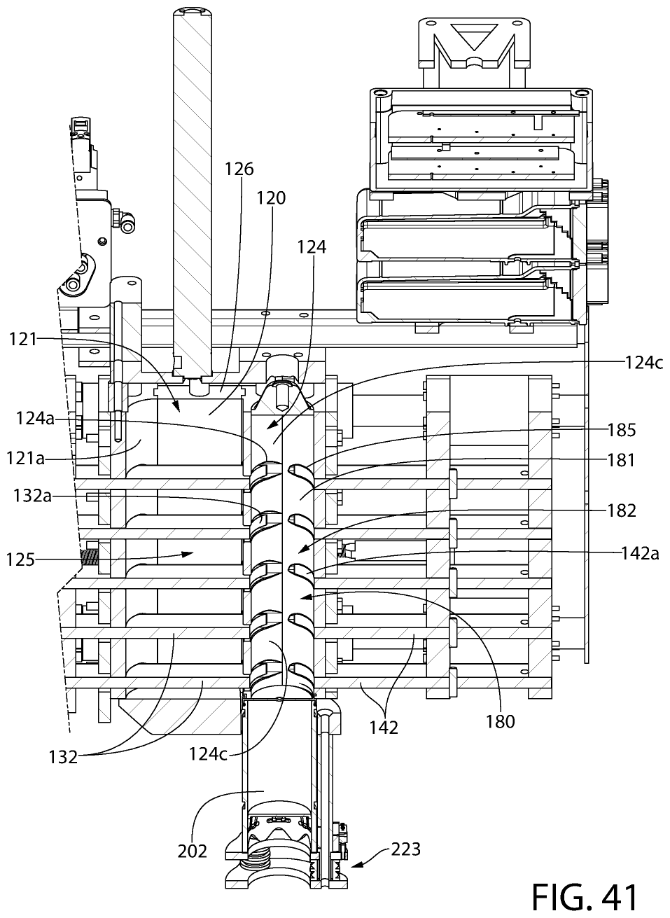

Bulk material chamber 120 has a vertically-extending hollow body comprising sidewalls 121 defining an internal cavity 125, a top 126, and a bottom 127 (see, e.g., FIG. 24). The bottom 173 of funnel 170 is coupled to cavity 125 through the sidewalls 121 of chamber 120 to introduce the bulk soil sample material. In one embodiment, chamber 120 has an oblong transverse cross-sectional shape which is horizontally elongated in the axial longitudinal direction parallel to longitudinal axis LA (see, e.g., FIGS. 41-43). Sidewalls 121 may comprise an arcuately curved wall section 121a opposite the flat wall side 124b of die block 124 which forms part of the sidewalls of chamber 120. Wall section 121a comprises a set of vertically spaced apart blade slots 128 extending completely therethrough to slideably receive sample blades 131 of the sample blade mechanism 130. Slots 128 are positionally synchronized with and diametrically opposite die slots 124b such that each slot 128 is horizontally axially aligned with a corresponding die slot 124a.

Sample collection chamber 180 has a cylindrical configuration and vertically-extending hollow body comprising sidewalls 181 defining an internal cavity 182, a top 183, and a bottom 184. In one embodiment, chamber 180 has a circular transverse cross-sectional shape. Sidewalls 181 comprise a set of vertically spaced apart blade slots 185 extending completely therethrough to slideably receive cleaning blades 141 of the cleaning blade mechanism 140. Slots 185 are positionally synchronized with and diametrically opposite die slots 124b in curved wall side 124c of die block 124 such that each slot 185 is axially aligned with a corresponding die slot 124b.

Sample blade mechanism 130 comprises a chassis 131 and plurality of sample blades 132 fixedly coupled to the chassis in a vertically spaced apart array. Chassis 131 may include a pair of horizontally spaced apart right and left vertical end plates 133 at each end. Sample blades 132 may be slideably coupled to the left end plate 133a of the pair and slideably pass through complementary configured holes in a right end plate 133b. The right and left end plates are each rigidly connected together by horizontally oriented tubular guide rod sleeves 134a so that the chassis moves right and left as a single unit with the sample blades 132 and end plates 133.

Chassis 131 is slideably carried by a plurality of guide rods 134 each spanning laterally between and coupled to the vertical end supports 112 of frame 111 at one end and the chamber block 123 located therebetween at the other end of the rods. Guide rods 134 are slideably received inside the hollow tubular guide rod sleeves 134a of the chassis described above. Two sets of four guide rods 134 in each set may be provided in one embodiment which are vertically and horizontally spaced apart from each other top to bottom and front to rear as shown. One set is for slideably supporting the sample blade chassis 131 and the other is for slideably supporting sample transfer blade chassis 141. Each set of guide rods 134 may include a pair of upper and lower guide rods; each of which have an associated guide rod sleeve 134a of chassis 131 and guide rod sleeve 144a of chassis 141.

Sample blades 132 may each have a horizontally elongated bar-like body terminated by a concave terminal end 132a which passes through bulk material chamber 120 and die block 124 during the tube filling operation. End 132a has a radius of curvature (concave) which corresponds to the radius of curvature of the sample collection chamber 180 to form part of the walls of the chamber when inserted through die block 124, as further described herein. In some embodiments, the blade bodies may be arcuately curved from side to side between the longitudinal long sides of the blade 132. In other embodiments, the blade bodies may be flat. Sample blades 132 are axially and laterally moveable in the horizontal longitudinal direction between a retracted position withdrawn from bulk material chamber 120 and die block 124, and a projected position extending through the chamber 120 and die block 124. When projected, the blades extract and extrude/push soil sample material blanks or plugs from the bulk material chamber 120 through the die block 124 and into the sample collection chamber 180. In the retracted position, the concave ends 132a of blades 132 are flush with and form part of the arcuate curved wall section 121a of the bulk material chamber 120.

Sample blade mechanism 130 comprises an actuator 135 which linearly moves the chassis 131 and array of sample blades 132 supported thereby between the retracted and projected positions. Actuator 135 may be any suitable commercially-available electric linear rod actuator, pneumatic actuator, or hydraulic actuator with retractable/extendible operating rod 136 coupled to the chassis 131.

Sample blades 132 may be vertically stacked and spaced apart in one embodiment as shown. The chassis 131 to which they are mounted moves all the sample blades in unison between the retracted and projected positions.

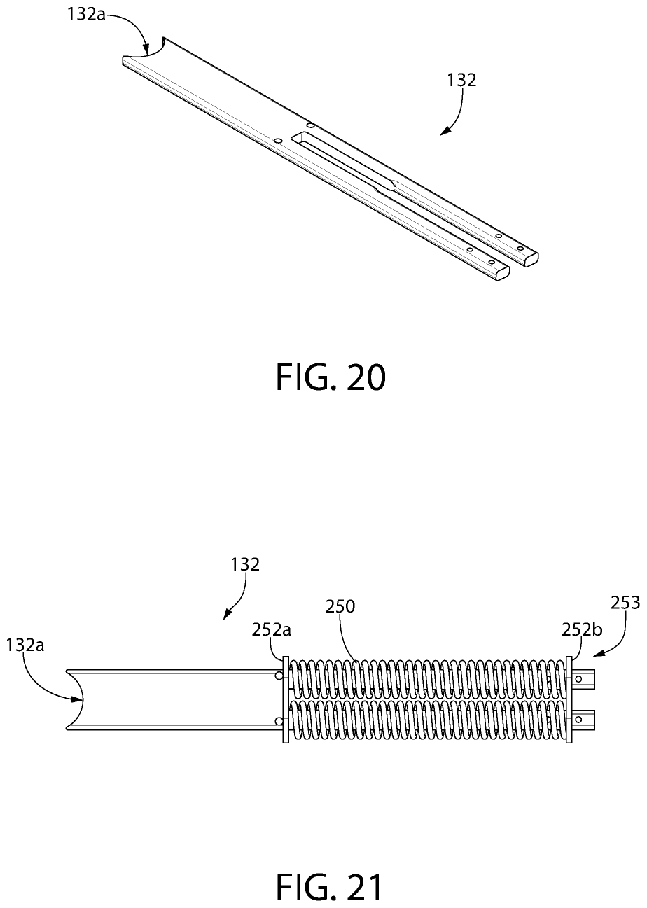

According to one aspect of the sample blade mechanism 130, a spring-biased protection system may be provided to protect the sample blades 132 and mechanism from damage in the event one or more die slots 124a in die block 124 are plugged by foreign debris such as stones/rocks wedged into the die slots from a previous sample material extrusion run. In one embodiment, each sample blade 132 is mounted in a corresponding blade spring pack 253 comprising a pair of springs 250. Referring to FIGS. 20-23, the distal end portion of blades 132 (farthest from bulk material chamber 120) is bifurcated into a pair of longitudinally elongated legs 251; each of which captures a coiled compression spring 250 therearound. The proximal end of each spring 250 may be braced against and acts on an elongated proximal spring plate 252a slideably coupled to the blade 132 between its distal and proximal end. Proximal spring plate 252a abuttingly engages right end plate 133b of chassis 131 when mounted therein. The remaining distal end of each spring may be braced against a second elongated distal spring plate 252b which abuttingly engages the left end plate 133a of chassis 131. The bifurcated distal end of the blades 132 are projectible through corresponding openings in left distal spring plate 252b and left end plate 133a of the chassis in the event the blade encounters blockage of its respective die slot 124a in die block 124 when the sample blade mechanism 130 attempts to insert the blade through the slot. In operation, the remaining blades 132 whose paths are not blocked in die block 124 will continue to advance into the bulk material chamber 120 to extract soil sample material while the blocked blade will remain stationary as the chassis moves towards chamber 120 to the right. The springs associated with the blocked blade or blades 132 will be compressed and the blocked blade will not advance any farther towards the bulk material chamber. Advantageously, the sample blade mechanism will continue to function and extract samples even in the event one or more sample blade paths are blocked so that the soil sample material packaging operation is not interrupted.

The spring force (k) of springs 250 is selected to allow the sample blades 132 to be inserted through the die slots 124a of die block 124 with a reasonable maximum amount of force determined to prevent damage to the blades and cleaning blade mechanism 140. When this predetermined maximum force limit is exceeded as a blade encounters a blocked or plugged die slot, the springs 250 will compress to arrest progress of the blade and prevent damage to the blade and the mechanism.

Cleaning blade mechanism 140 may be generally similar to the sample blade mechanism 120 in construction and operation; however, the cleaning blades 142 enter the sample collection chamber 180 and die block 124 from the opposite direction to removed and clean residual soil deposits from the die block slots 124a in preparation for the next tube filling operation. Briefly, without undue repetition, cleaning blade mechanism 140 also comprises a chassis 141 supporting the plurality of cleaning blades 142 fixed coupled to the chassis in a spaced part array. Chassis 141 is slideably carried by the same type hollow tubular guide rod sleeves 144a which travel on guide rods 144 spanning laterally between and coupled to the vertical end supports 112 of frame 111 and chamber block 123. Chassis 141 also includes a pair of horizontally spaced apart vertical end plates 143. Cleaning blades 142 may be fixedly coupled to a right end plate 143a of the pair and slideably pass through complementary configured holes in a left end plate 143b. The blades may optionally be fixedly connected to the left end plate as well in some embodiments.

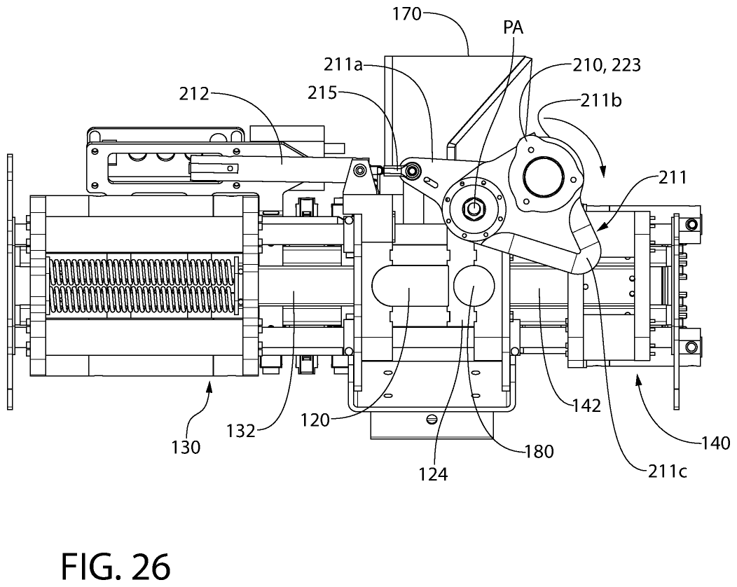

It bears noting that the cleaning blades 142 preferably do not include a spring protection mechanism like the sample blades 132. Since the function of the cleaning blades includes dislodging any debris (e.g., stones/rocks, hardened agricultural clumps, etc.) from the die block die slots 124a, it is preferred that the cleaning blades can be advance with full force through the die slots in a rigidly supported manner. It is therefore desirable in some embodiments for the cleaning blades 142 to not have the spring-operated relieving feature. If the cleaning blades are unable to forcefully dislodge an object obstructing one or more die slots 124a, the sample extraction cycle will stall (e.g., cleaning blade mechanism 140), which is detected by sample packaging machine controller 2811 via an appropriately configured sensor 2811c (see, e.g., FIG. 26) configured to detect that the sample blades have not fully advance and been inserted in the die slots. A positional or proximity sensors may be used for this purpose of the types similar to full tube sensor 220 described elsewhere herein, or other type sensors. The controller will electronically “time out” the cycle, indicating to the operator there is an internal problem. If a foreign object were allowed to stay lodged in the die block and the extraction cycle continues, damage will result to internal components of the apparatus mechanism. The controller 2811 communicates (e.g., visually and/or audibly) the “time out” condition to the human operator via an appropriate alarm signal so the operator is made aware of and can remedy the die block obstruction issue to restore normal operation.

Cleaning blades 142 may have the same shape as sample blades 132 at least in cross section since they pass through the same horizontally elongated slots 124a in the die block 124 to remove residual soil therefrom, as further described herein. FIGS. 20 and 21 showing sample blades 132 may also represent cleaning blades 142 in shape and construction except the cleaning blade do not bifurcated ends because not spring protection mechanism is provided. Cleaning blades 142 also may each have a horizontally elongated bar-like body terminated by a concave terminal end 142a which passes through sample collection chamber 180 and die block 124 during the die cleaning operation. In some embodiments, the blade bodies may be arcuately curved between the longitudinal long sides. In other embodiments, the blade bodies may be flat. Cleaning blades 142 are axially and laterally moveable in the horizontal longitudinal direction between a retracted position withdrawn from chamber 180 and die block 124, and a projected position extending through the chamber 180 and die block 124. This pushes residual soil in slots 124a of die block 124 back into the bulk material chamber 120 from which they can be purged. In the retracted position, the concave ends 142a of blades 142 are flush with and form part of the arcuate curved sidewalls 181 of the sample collection chamber 180.

Cleaning blade mechanism 140 comprises an actuator 145 which linearly moves chassis 141 with the array of cleaning blades 142 supported thereby between the retracted and projected positions. Actuator 145 may be any suitable commercially available electric linear rod actuator, pneumatic actuator, or hydraulic actuator with retractable/extendible operating rod 146 coupled to the chassis 141.

Cleaning blades 142 may be vertically stacked and spaced apart in one embodiment as shown. The chassis 141 to which they are mounted moves all the cleaning blades in unison between the retracted and projected positions. The cleaning blades 142 are positionally synchronized with the die slots 124a such that each blade is axially and horizontally aligned with a corresponding one of the die slots.

The sample and cleaning blades 132, 142 as well as the structural components of the sample and cleaning blade mechanisms 130, 140 (e.g., end plates, guide rods and sleeves, etc.) described above preferably are made of a suitably strong metallic material, such as steel or stainless steel in some embodiments. Other suitable materials may be used.

Compaction piston-plunger 150 and sample transfer piston-plunger 155 may each be any suitable type commercially available electric linear rod actuator, pneumatic cylinder, or hydraulic cylinder with retractable/extendible operating rod 151a and 151b. Rods 151a, 151b are terminated with a disk 152a, 152b respectively; each being configured to fit closely and conform to the shape and internal diameters of the bulk material chamber 120 and sample chamber 180 without any appreciable gaps between the disks and chamber walls. Disks 152a, 152b are sized such that the peripheral edges of the disks are located adjacent to their respective chamber walls to scrape any soil away from the walls as the disks slide upwards/downwards in their chambers. Disk 152a of compaction piston-plunger 150 has an oblong transverse cross-sectional shape which corresponds to the oblong cross-sectional shape of bulk material chamber 120. Disk 152b of sample transfer piston-plunger 155 has a circular transverse cross-sectional shape which corresponds to the circular cross-sectional shape of the sample collection chamber 180.

Piston-plungers 150, 155 are each vertically oriented in one embodiment and enter the open tops of chambers 120, 180. In operation, the piston-plungers are each linearly moveable from a retracted upward position withdrawn from chambers 120 or 180, or projected downward position extending to the bottom end of the chambers. The exposed bottom surfaces of each disk 152a, 152b may be substantially flush with the bottom end faces of the chambers 120, 180 to ensure all soil is purged from the chambers and to allow the lower disk faces to be scraped/wiped clean via operation of the carousel, as further described herein.

Funnel 170 provides a hopper-type vessel into which the bulk soil sample is filled for transfer to the bulk material chamber 120. Housing 116 includes an annular chute 170a positioned over and optionally detachably coupleable to and partially insertable into the top of the funnel for guiding the agricultural sample material (e.g., soil or other) into the funnel. Chute 170a may comprise sloping walls in some embodiments as shown to facilitate adding the sample material to the funnel. In certain embodiments, the sloping walls of funnel 170 and chute 170a may be comprised of a nonstick material such as UHMW-PE or similar, and/or be coated to encourage sample material to flow through and not adhere to the walls. This may be particularly beneficial when processing sticky type soils such as clay.

The funnel 170 may have any suitable configuration and gradually narrows in width from the open top 174 for adding the soil sample to the funnel to the bottom 173 under control of an openable/closeable funnel door 171 coupled to door actuator 172. In one embodiment, funnel 170 may be trapezoidal shaped as shown. Other funnel shapes including frustoconical shaped funnels may be used. Funnel door 171 is pivotably and hingedly mounted to the lower portion of the funnel via hinge pin 171a and coupled to an eccentric cam lever 175 operably coupled to actuator 172. Funnel door 171 is pivotably moveable via operation of actuator 172 between a closed position for filling the funnel 170 with the bulk soil sample material, and an open position for transferring the soil to bulk material chamber 120. The actuator 172 may be any suitable commercially available electric linear rod actuator, pneumatic actuator, or hydraulic actuator with retractable/extendible operating rod 176 coupled to the cam lever 175.

Container carousel 200 is configured to removably hold and rotatably move the sample container 201 inwards under the sample chamber 180 and outwards therefrom during the soil sample packaging operation. Carousel 200 is rotatably mounted about a vertical pivot axis PA under packaging apparatus 110 and positioned below and proximate to the bottom ends of the bulk material chamber 120 and sample chamber 180. The carousel generally includes a rotatable base member 211 supporting a container holder 210 and carousel actuator 212 coupled to the base member. The base member 211 in one embodiment may comprise a generally broad and flat plate-like monolithic structure having a compound configuration including outwardly extending eccentric cam arm 211a, container support arm 211b, and a solid closure arm 211c. Pivot axis PA is formed by a pivot pin 216 extending vertically through the base member 211 and offset from the geometric center of the plate. Base member 211 is rotatably movable about pivot axis PA in a horizontal reference plane defined by pivot pin 216 which mounts the base member to the support frame 111.

Solid closure arm 211c is rotatable underneath and adjacent to the normally open bottom end of bulk material chamber 120 to selectively close or open the bottom end of the chamber via rotation of base 211, as further described herein. The top surface of at least the closure arm 211c is preferably flat to form relatively tight closure of chamber 120 at bottom to prevent the bulk soil from falling through the chamber. Closure arm 211c slideably moves into and out of position against the bottom end of chamber 120. This advantageously helps clean and remove any soil residue at the bottom of the chamber 120 between tube filling cycles via a wiping/scraping type action across the bottom face of the chamber.

Container holder 210 may be a generally cup-shaped structure in one embodiment supported by base member 211. In one non-limiting embodiment, the holder includes a circular opening formed in the container support arm 211b of the base member to receive sample tube 202 and a circular support disk 213 suspended below support arm 211b in a vertically spaced apart manner by a plurality of vertically elongated spacer rods 214. The container holder 210 is configured via positioning of support disk 213 such that a majority of the height of the sample tube 202 is located below the base member 211 as shown. This ensures that the tube is held by the container holder in a stable manner both during the tube sample filling operation and during rotation of the tube with the base member 211. Other forms and configurations of container holder 210 are possible so long as the sample tube 202 can be removably held with the required degree of stability.

Sample tube 202 may be configured to be rotationally locked in position to support disk 213 to ensure the tube does not rotate and remains rotationally stable during the tube filling operation and movement of the base member 211 of carousel 200. In one embodiment, the bottom end 203b of the cylindrical walls 202a of tube 202 may have an arcuately undulating castellated configuration to form a rotational interlock with a mating arcuately undulating castellated feature on the support disk 213. Accordingly, the bottom end 203b of sample tube 202 adjacent to where retention slots 202c are formed in the tube may comprise anti-rotation protrusions 206a which lockingly engage a ring of mating anti-rotation protrusions 206b on the tube support disk 213. The protrusions 206a, 206b may have rounded ends with sloping sides in some embodiments as shown to help guide the sample tube 202 onto the castellated support disk 213. In other embodiments, the anti-rotations features may be omitted where it might not be necessary or desirable to rotationally lock the sample tube 202 into the packaging apparatus 110 during the filling operation.

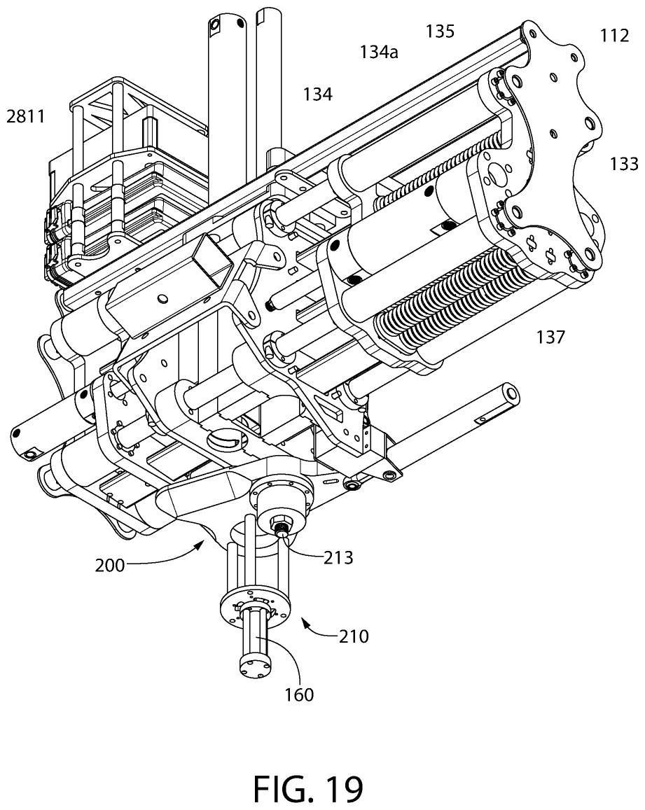

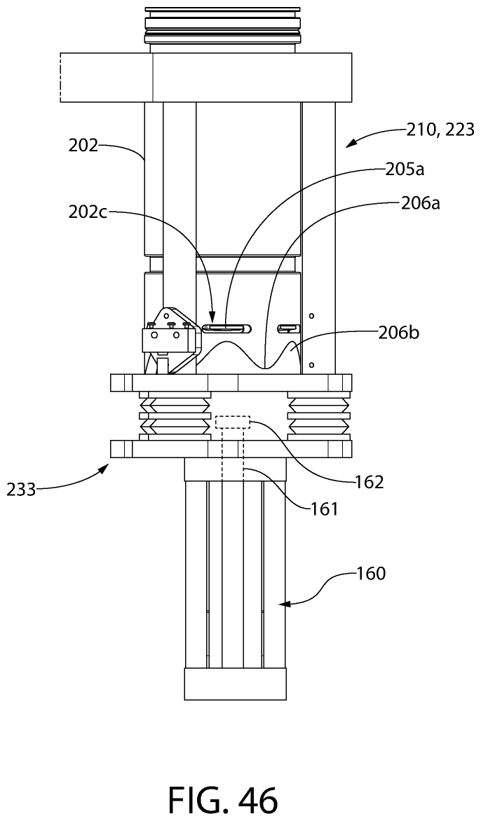

In one embodiment, container end cap actuator 160 may be mounted to support disk 213 of container holder 210 (FIG. 19), or alternatively collapsible dual support plate assembly 233 of alternative container holder 223 (see, e.g., FIG. 46). Cap actuator 160 is therefore rotatable with base member 211 of carousel 200. The cap actuator 160 may be any suitable commercially available electric linear rod actuator, pneumatic actuator, or hydraulic actuator with retractable/extendible operating rod 161 which is projectable through a complementary configured opening in the support disk 213 to engage and activate the push cap 204b. Rod 161 may be terminated with a with a diametrically enlarged cylindrical disk 162 configured to engage push cap 204b. Actuator 160 is operable to disengage and unlock the push cap from retention slots 202c (i.e. retention protrusions 205a) so that the cap can be moved upwards in sample tube 202 for reasons associated with the sample tube filling operation, as further described herein.

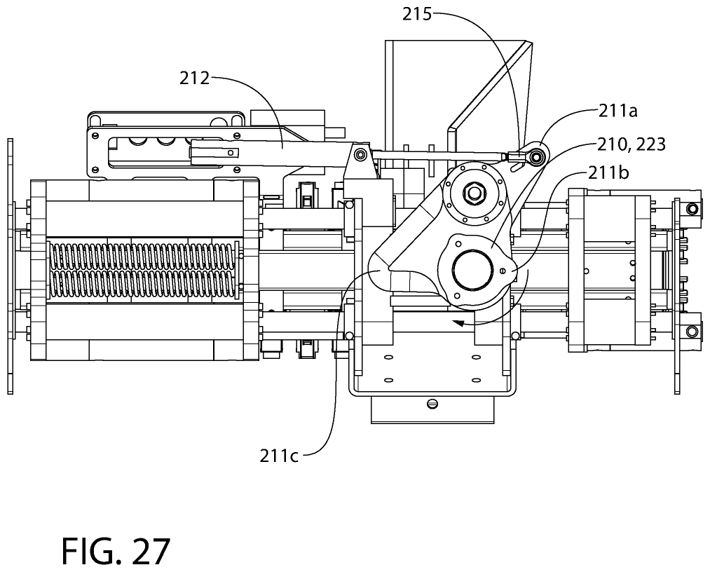

The actuator 212 coupled to cam arm 211a of base member 211 may be any suitable commercially available electric linear rod actuator, pneumatic actuator, or hydraulic actuator with retractable/extendible operating rod 215 coupled to the cam arm (see, e.g., FIG. 26). Actuator 212 is operable to rotate base member 211 about pivot axis PA to move the carousel between: (1) an inward closed position for tube filling in which sample tube 202 is positioned directly beneath and adjacent to the bottom end of sample chamber 180 and base member closure arm 211c is positioned directly beneath and adjacent to the bottom end of bulk material chamber 120 to close that chamber (see, e.g., FIG. 27); and (2) an outward open position in which sample tube 202 is not positioned beneath sample chamber 180 and base member closure arm 211c is not positioned beneath and adjacent to the bottom end of bulk material chamber 120 which is then downwardly open (see, e.g., FIG. 26). In the outward position, sample tubes 202 may be exchanged between tube filling cycles to remove filled tubes and mount new empty tubes.

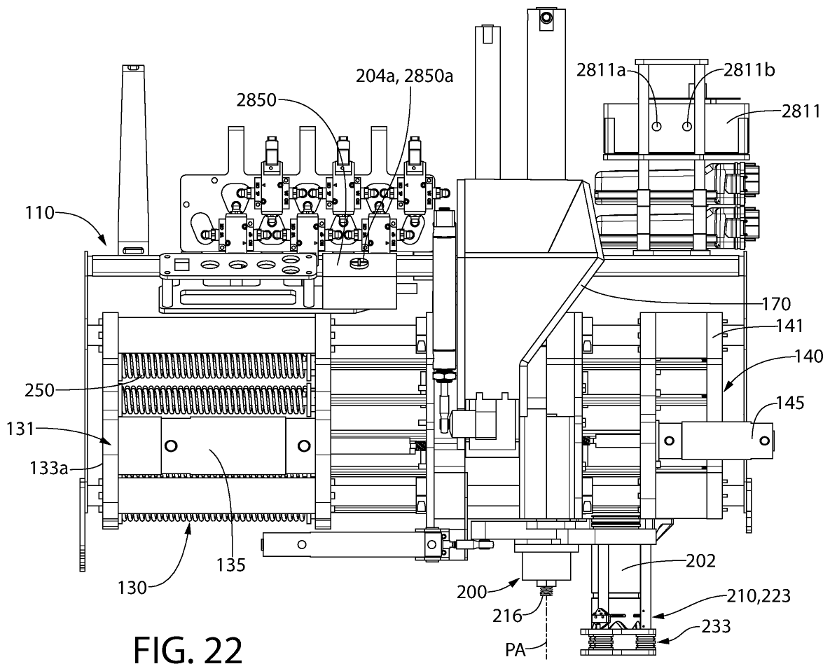

A method or process for packaging an agricultural sample will now be described. In one embodiment, the sample may be a soil sample which will be used for convenience and without limitation as a basis for describing the operation of agricultural sample packaging system 100 disclosed herein. FIG. 1 is a high-level process flow chart providing a summary overview of the packaging process. FIGS. 22-40 are sequential images showing one non-limiting embodiment the sample packaging operation and positional changes of the agricultural sample packaging apparatus 110 described herein. The sample material used in the process below may be soil for convenience of discussion and not limitation.

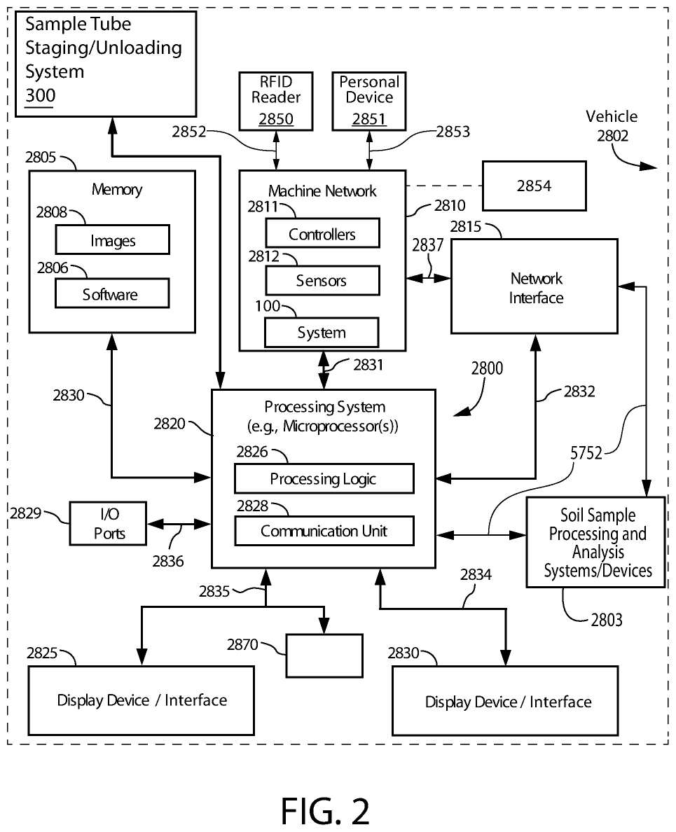

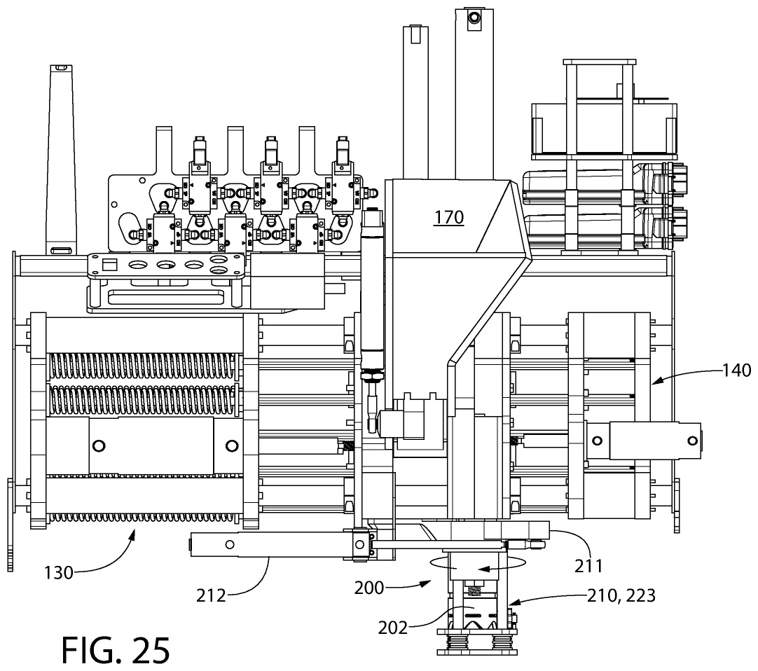

The method begins with the carousel 200 in the outward open position (see, e.g., FIG. 22). If the carousel is not outward and ready to accept the new empty sample tube 202, a carousel may be rotated outwards by actuating an actuator 2811b such as a button, toggle, or other type switch (FIG. 22) in some embodiment which is operably coupled to packaging machine controller 2811 (see, e.g., FIGS. 25-27). This may arbitrarily be referred to as the “Load Tube” switch or by another name. In other embodiments, the step may be actuated via a personal electronic device 2851 operably coupled to controller 2811 which initiates outward rotation of the carousel (see, e.g., FIG. 2). In either control scenario, rotating the carousel outward enables the operator to manually insert sample tube 202 into the container holder 210 or 223 of the carousel 200.

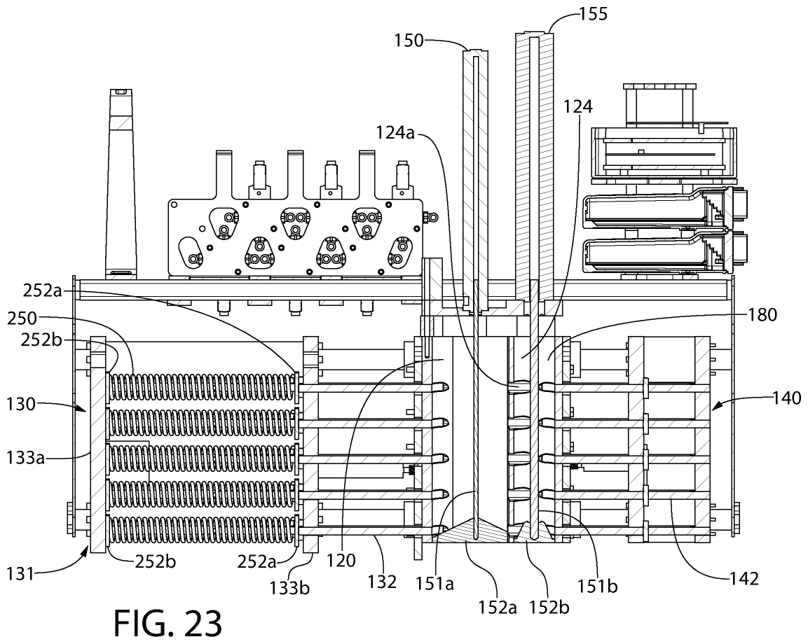

At the start of the packaging operation, the piston-plungers 150 and 155 are preferably in their downward extended/projected position shown in FIG. 23 from either the previous container packaging operation or in preparation to start the first packaging operation.

An empty sample container 201 such as sample tube 202 is first vertically inserted into and placed in the container holder 210 of the carousel A castellated and rotationally interlocked interface is formed between the bottom end 203b of the tube and support disk 213 of the holder 210, as previously described herein. The top end cap 204a of sample tube 202 may contain a tracking tag such as RFID tag 2850a in one non-limiting embodiment which is placed on RFID reader 2850. Packaging machine controller 2811 and/or main system controller 2820 automatically reads the tag and begins tracking the sample including all relevant data such as geolocation via GPS sensor 2854, time of day, etc. The RFID tag could alternatively be contained within the plunger cap 204b or cylindrical wall 202a of the sample tube and read automatically when the sample tube is loaded into the carousel 200. Additionally, if the RFID tag is within any portion of the tube or plunger cap that is loaded into container holder 210, the reader may be attached to container holder 210, enabling the capability to read the tube when it is loaded and not requiring a separate station for RFID reading.

At this point in the method/process, the funnel door 171 is in the closed position (see, e.g., FIG. 24). This prevents the soil sample material if loaded into the funnel 170 from entering and falling out of the still open bottom of the bulk material chamber 120 if loaded into the funnel before the sample tube 202 is rotated beneath chamber 120.

Carousel 200 is then rotated to the inward closed position via operation of carousel actuator 212. In some embodiments, this step may be initiated by actuating an actuator 2811a such as a button, toggle, or other type switch (FIG. 22) operably coupled to packaging machine controller 2811 (see, e.g., FIGS. 25-27). This may arbitrarily be referred to as the “Fill Tube” actuator or by another name. In other embodiments, the step may be actuated via a personal electronic device 2851 operably coupled to controller 2811 which initiates rotation of the carousel (see, e.g., FIG. 2). In either control scenario, rotating the carousel inwards positions sample tube 202 under sample collection chamber 180. The base member 211 (i.e. closure arm 211c) closes the bottom end of previously open bulk material chamber 120.

With the piston-plungers 150, 155 still in their downward position, it bears noting that the closure arm 211c of the carousel 200 will scrape any residue away from the bottom faces of the plunger disks 152a, 152b as the closure arm rotates inwards which may remain from the last container packaging operation.

The soil sample material may be added to funnel 170 after rotating the carousel inwards or at any point before that so long as the funnel door 171 remains closed up to this point in the process.

Next, the push cap 204b is unlocked/unclipped from the sample tube 202 by actuating end cap actuator 160 for those embodiments including the actuator. The actuator disengages the retention protrusions 205 of push cap 204b from the retention slots 202c in tube 202 and slideably pushes the cap vertically upwards inside the tube. Cap 204b is therefore no longer locked near the bottom end 203b of sample tube 202, but at some preselected position between the ends of the tubes. The protrusions 205 have a radially outwards directed spring force sufficient to positively engage the inside surface of cylindrical sample tube wall 202a to retain the position of the push cap 204b.

Alternatively, if the alternative container holder 223 embodiment without actuator 160 is used as further described below and shown in FIG. 22 et al., the push cap remains at its lower locked position in sample tube 202.

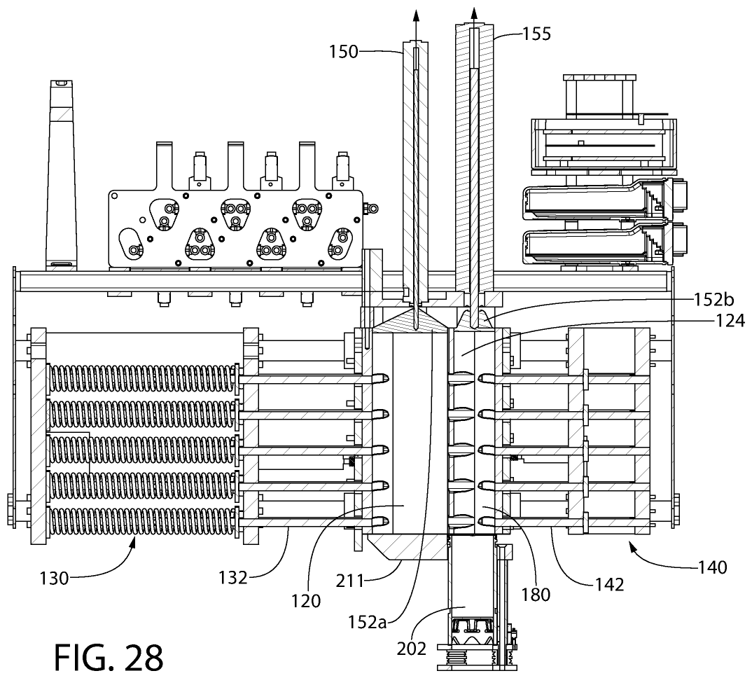

As shown in FIG. 28, the piston-plungers 150, 155 are retracted from the bulk material chamber 120 and sample collection chamber 180 (see, e.g., FIG. 28) if not previously retracted.

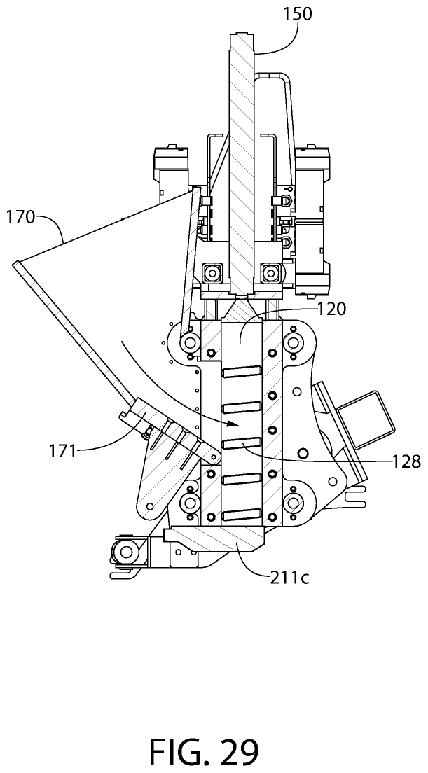

Once the carousel is set up as described above and piston-plungers 150, 155 are retracted, the soil sample extraction process is ready to begin. The funnel door 171 is opened via operation of actuator 172. The bulk soil sample material is transferred to and enters the bulk material chamber 120 (FIG. 29). Door 171 may then be shut, which compacts any slight overfill of the chamber and prevents any remaining soil in the funnel from working its way into the chamber.

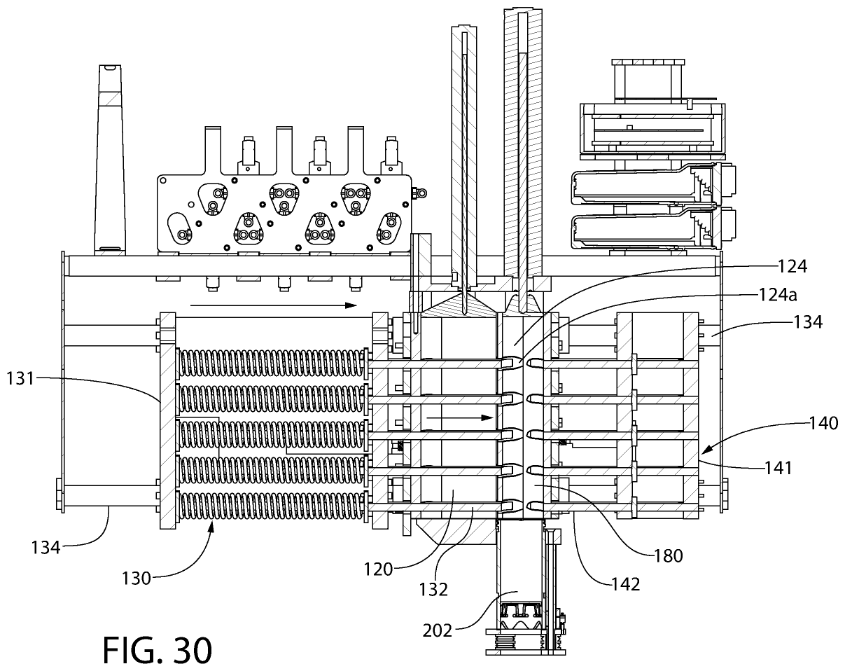

The method continues with activation of the sample blade mechanism 130. Actuator 135 slideably moves the sample blade chassis 131 along guide rods 134 and sample blades 132 are inserted through bulk material chamber 120 and die block 124. The blades 132 move from the initial retracted position to the projected position previously described herein (e.g., towards the right in FIG. 30—see directional arrows). The sample blades 132 each extrude or force a portion of soil from the bulk material sample through a respective die slot 124a in die block 124. The extruded/extracted soil, which comprises multiple plugs or blanks of soil are deposited in sample collection chamber 180 by the advancing blades 132). The extracted soil plugs fall downward into open sample tube 202 waiting below chamber 180.

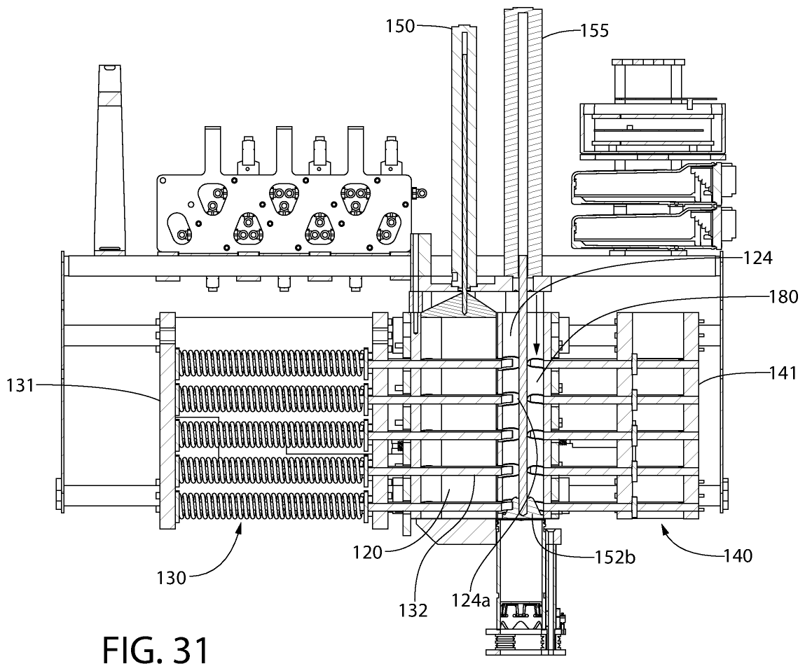

Sample transfer piston-plunger 155 may then be actuated to ensure all of the soil sample material in collection chamber 180 is positively pushed downwards into the sample tube (see, e.g., FIG. 31). Preferably, this step is conducted while sample blades 132 remain in the die block 124 within die slots 124a to ensure that the soil in chamber 180 is not pushed laterally backwards into the die slots 124a of die block 124 by the piston-plunger. Piston-plunger 155 may be extended all the into the sample tube 202 to tightly pack the soil in the tube. Notably, the piston-plunger compacts the soil in sample tube 202 while forcing and displacing the push cap 204b back downwards incrementally by a distance. This advantageously ensures that soil sample in the tube is a tightly packed sample.

Piston-plunger 155 may then be withdrawn from the sample tube 202 and chamber 180, and returns vertically upwards back to its initial retracted position.

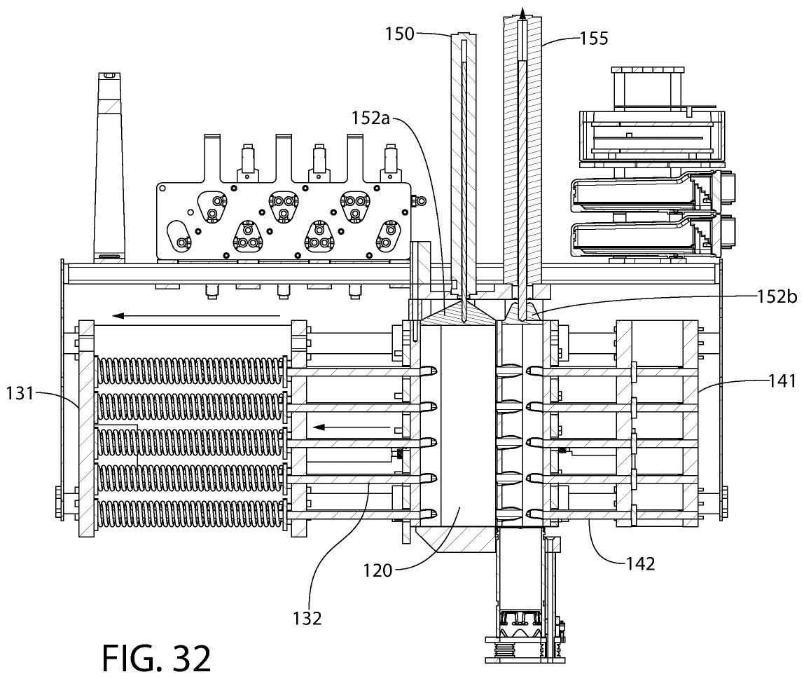

The method continues with withdrawing the sample blades 132 from the die block 124 and bulk material chamber 120 by returning the chassis 131 of the sample blade mechanism 130 to the retracted position (e.g., towards the left in FIG. 32).

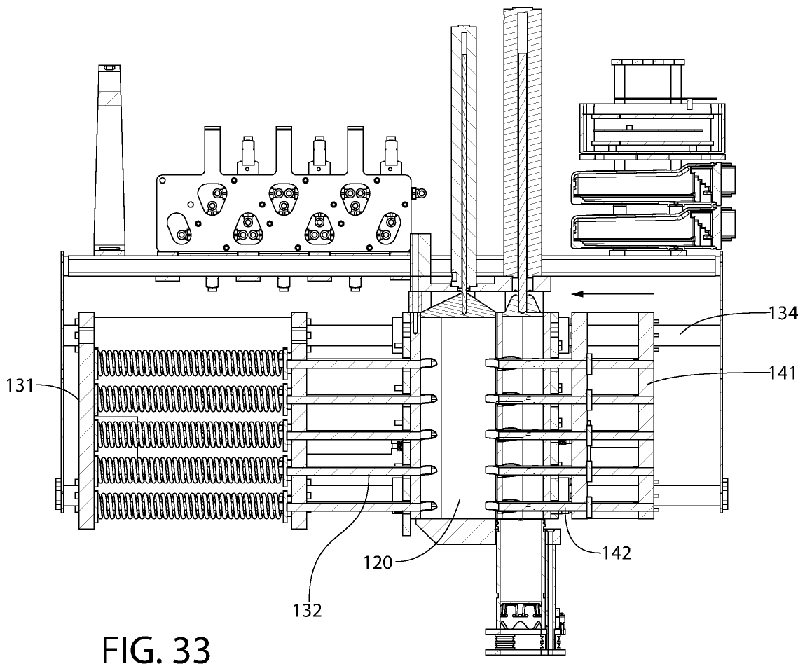

The method continues with actuating the cleaning blade mechanism 140. Actuator 145 moves the cleaning blade chassis 141 and cleaning blades 142 from the initial retracted position to the projected position previously described herein (e.g., towards the left in FIG. 33). Cleaning blades 142 are inserted through sample collection chamber 180 and die slots 124a in the die block. The cleaning blades 142 each push any residual soil residue or particularly hard non-soil debris (e.g. stones, etc.) which may have been trapped in the die slots 124a backwards and into the bulk material sample chamber 120.

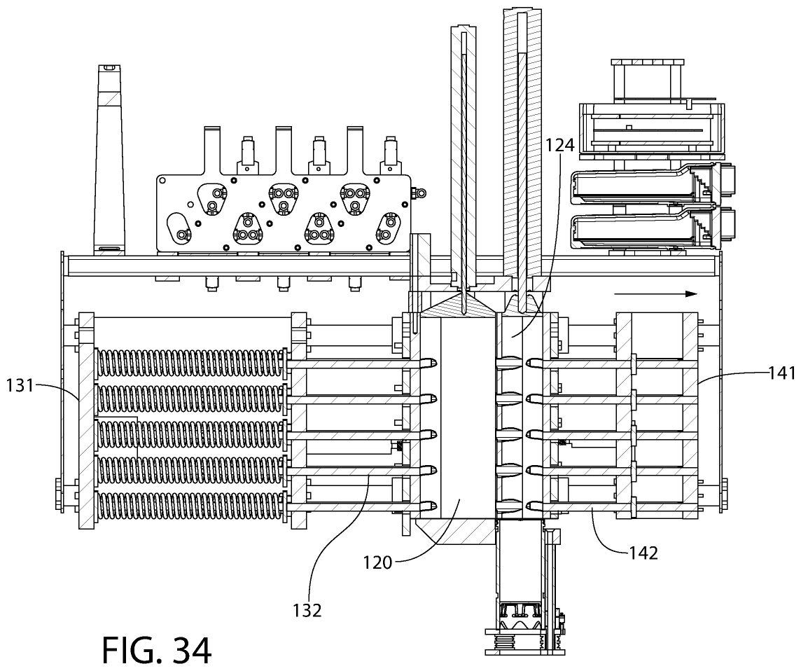

The cleaning blades 142 are then retracted from the die block 124 and soil collection chamber 180 by reversing the above operation (see, e.g., FIG. 34).

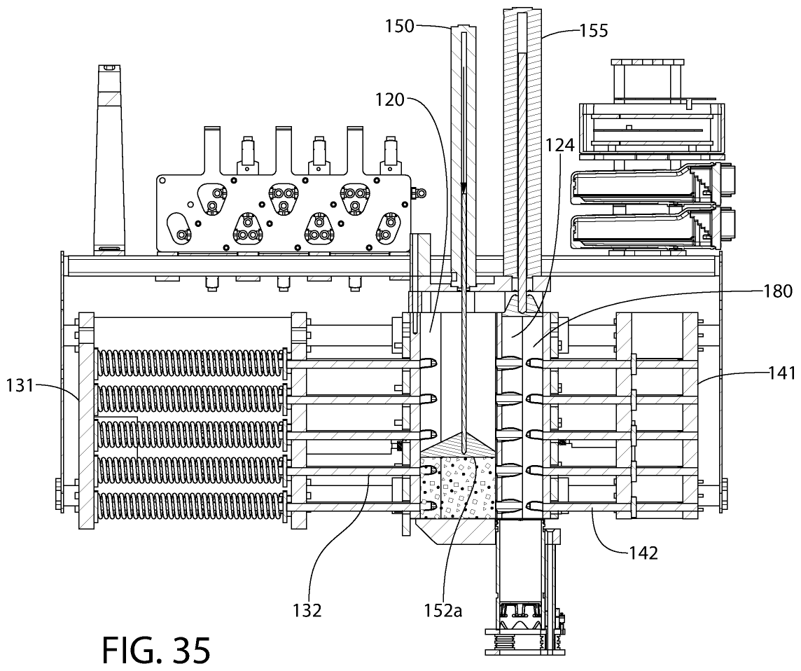

Next, compaction piston-plunger 150 is actuated to re-pack the soil in bulk material chamber 120 (see, e.g., FIG. 35). The piston-plunger 150 moves vertically downwards in chamber 120 to compact the remaining soil in the chamber. This is particularly beneficial if moist sticky and dense soil such as clay is being processed. It is possible that the sample blades 132 may leave voids in the self-supporting soil material remaining in chamber 120 when the blades are initially withdrawn from the soil in the chamber when the sample material plugs were first extruded. If so, the next extraction cycle may fail to displace and collect any appreciable amount of soil for the sample due to the presences of the voids. To remedy this possibility and maintain a quick pace of extracting a full soil sample, the piston-plunger 150 advantageously will collapse the soil and remove any such voids.

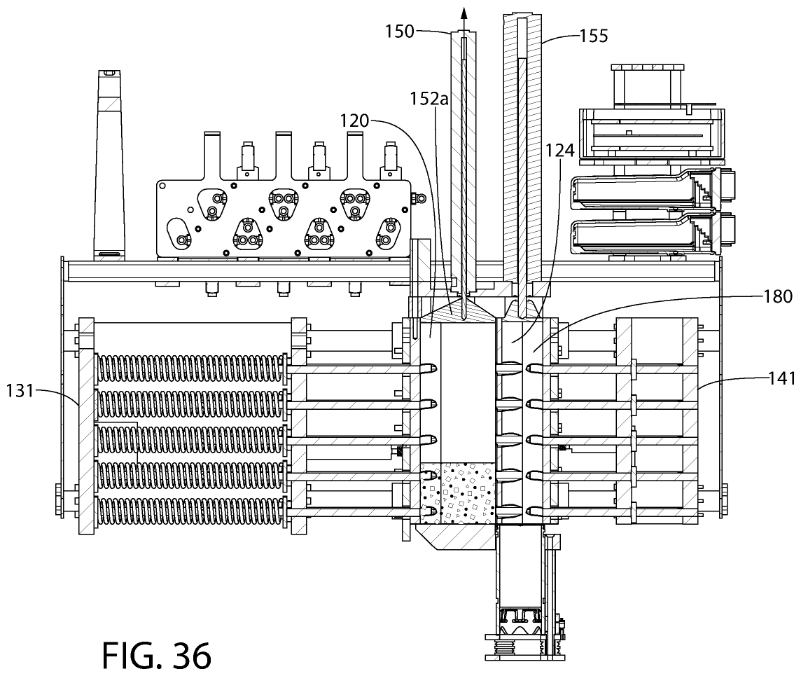

Next, the compaction piston-plunger 150 is retracted from bulk material chamber 120 (see, e.g., FIG. 36).

It bears noting that a single soil sample plug or blank extraction cycle as described above may not be sufficient to sufficiently fill the sample tube 202 with enough soil for further processing and eventual chemical analysis. Accordingly, the entire cycle may be repeated multiple times to extract a sufficient amount of the soil sample material for chemical testing in a related process.

It should also be noted that in sample packaging apparatus 110 embodiments including the cap actuator 160, the push cap 204b gradually advances farther downwards inside sample tube 202 as the tube is gradually filled and packed down by piston-plunger 155 as previously described herein. The retention protrusions 205 of cap 204b eventually re-engage and snap back into the retention slots 202c of the sample tube. This represents the lowermost position of the cap 204b in the tube and maximum volumetric capacity of the tube.

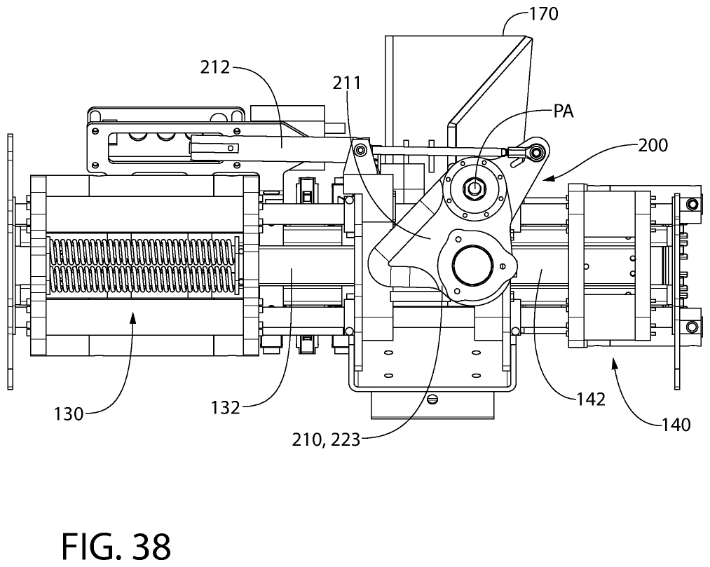

Once the sample tube 202 is full, the carousel 200 is rotated back to the outwards open position (see, e.g., FIGS. 38-29). The carousel is configured such that it screeds the top of the tube to remove any soil mounded above the top rim of the tube so that the top end cap 204a can be installed without having to manually level off any upwardly protruding mound of soil. Any mound of soil will be screeded or scraped off by the bottom surface of the chamber block 123 portion adjacent to sample collection chamber 180 as the baseplate 211 of the carousel 200 rotates to its open outward position. The stationary end cap 204a may then be manually removed from RFID reader 2850 with RFID tag 2850a and coupled to the open top end 203a of the tube to seal the sample. If the tag 2850a is not located on the stationary top end cap 204a, that cap need not be placed on an RFID reader and may be recovered from wherever stored. In either case, the packed and sealed sample tube 202 may then be removed from packaging apparatus 110 for further processing and eventual chemical analysis.

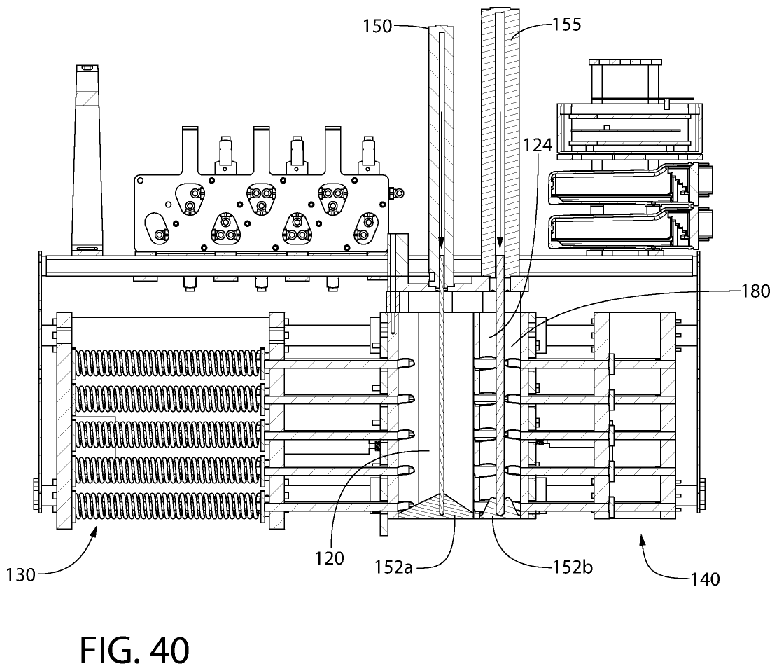

With the carousel still in the outward open position, sample transfer piston-plunger 155 and compaction piston-plunger 150 may be run back through their respective soil collection and bulk material chambers 180, 120 to remove any residual soil in preparation for the next round of extracting the soil samples (see, e.g., FIG. 40).

The sample transfer piston-plunger 155 and compaction piston-plunger 150 may remain in this downward position with disk 152a, 152b exposed at the bottom ends of the bulk material chamber 120 and sample collection chamber 180. After the sample tube 202 has been loaded into the carousel 200 for the next set of soil sample extractions, rotating the carousel to inward closed position will wipe the exposed faces of the piston-plungers 150, 155 clean as the closure arm 211c passes beneath both piston-plungers. The piston-plungers can each be returned to their upward position before soil is added to the bulk material chamber 120 for the sample plug/blank extractions.

It bears noting that compaction piston-plunger 150 doubles as a scraper to clean the ends of the sample blades 132 when in their retracted position each time the plunger moves downwards and upwards in the bulk material chamber 120. In a similar manner, the sample transfer piston-plunger doubles 155 which travels upwards/downwards in sample collection chamber 180 acts as a scraper to clean the ends of the sample blades 132 when in the projected position extending through the die block 124 and the ends of the cleaning blades 142 with each downward/upward movement of the plunger. This is possible because, as previously described herein, the terminal ends of the sample blades 132 form part of the exposed inner walls of the chamber 120 when retracted and part of the inner walls of chamber 180 when projected. In a similar vane, the terminal ends of cleaning blades 142 form part of the inner walls of chamber 180.

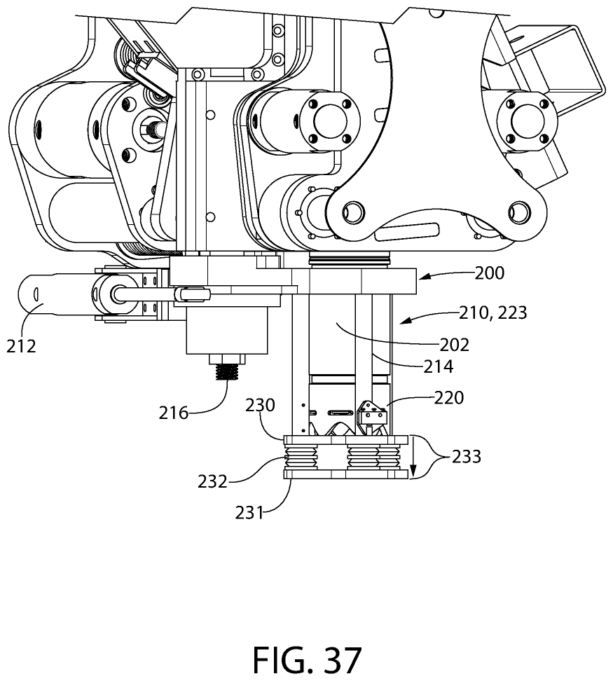

FIGS. 22 and 37 depict an alternative embodiment of a sample container holder assembly which omits the end cap actuator 160. In this embodiment, the present container holder 223 comprises a collapsible dual support plate assembly 233 which is supported and suspended by spacer rods 214 beneath the base member 211 of carousel 200. The dual plate assembly comprises a lower fixed plate 231 rigidly coupled to the bottom ends of spacers rods 214 and upper floating plate 230 vertically slideable up and down on the spacer rods relative to fixed plate 231. Fixed plate 231 thus remains stationary relative to the base member 211 of carousel 200 and spacer rods 214. The fixed and floating plates 231, 230 are spaced apart by plurality of springs 232 coupled between the plates. The sample tube 202 is seated on the upper floating plate when the tube is positioned on the packaging apparatus 110. The support plate assembly 233 is movable between an expanded condition in which the floating and fixed plates 230, 231 are spaced apart by springs 232 by a maximum extent and distal to each other, and a collapsed condition in which the plates 230, 231 are proximate to each other or closest when the springs are compressed by piston-plunger 155 as further described herein.

In operation, the general steps in foregoing description of the method for packaging an agricultural sample remain the same and will not be repeated for sake of brevity. The differences in the method when using the present container holder 223 is as follows. First, the push cap 204b is not unlocked and moved upward in sample tube 202 before adding soil to the tube since the cap actuator 160 is omitted in the present embodiment. Accordingly, cap 204b remains in its lowermost position proximate to bottom end 203b of tube and locked to the retention slots 202c of the tube. In some embodiments, the cap 204b could be replaced by a fixed immovable cap coupled to the bottom end of the tube similar to cap 204a coupled to the top tube end 203a.

A second difference in the method for packaging the agricultural sample is that the movable dual plate assembly 233 is activated and collapsed when the sample tube 202 is completely filled to capacity. The dual plate assembly 233 begins in the expanded condition when the sample tube is loaded onto the present container holder 223. After several tube fill cycles previously described herein, the tube will become full as the sample transfer piston-plunger 150 tamps and packs the soil into the tube. Once the maximum volume has been reached, the last tamping action will push the soil in the tube 202 and tube itself downwards together. This in turn moves the upper floating plate 230 downwards compressing the springs 232 counter to their upward biasing action. Support plate assembly 233 is now in the collapsed condition.

A full sample tube sensor 220 fixedly mounted adjacent to the bottom end of one of the spacer rods 214 is activated by the floating plate moving downward and configured detect movement of the floating plate, which is indicative of the tube being filled to capacity and ready for removal from the packaging apparatus 110. Sensor 220 sends a signal to the local machine controller 2811 onboard apparatus 110 and/or main system controller 2820 indicating the same. It bears noting that floating plate 230 will only be displaced when the tube 202 is filled to capacity as described above.

Any suitable type commercially available presence or contact sensing sensor including micro limit switches of suitable type, Hall effect sensors, etc. may be used to detect the presence or movement of floating plate 230. In one embodiment, a plunger type micro limit switch may be used as shown which comprises a depressible spring-biased plunger 221 that contacts upper floating plate 230 which the dual plate assembly dual support plate 233 is in the expanded condition (see, e.g., FIG. 46). If a micro limit switch is used, electrical contacts in the switch may be either opened (in a normally closed circuit) or closed (in a normally open circuit). In other embodiments, suitable load or force sensing sensors may be used. In yet other embodiments, non-contact type proximity sensors including inductive proximity, capacitive proximity, and photoelectric sensors may be used. Accordingly, there are a number of sensor options and the invention is not limited by the type of sensor employed so long as an appropriate “full tube” signal is transmitted to the local machine and/or main system controllers 2811, 2820.

It should be noted that the sample transfer piston-plunger 155 which tamps the sample into sample tube 202 does not directly apply force directly to the tube. Piston-plunger 155 applies its force directly to the sample in tube, and then the sample transmits its received force to the tube. This indirect force transfer allows the sample to be compressed consistently by the piston-plunger within the tube before triggering a full tube condition via sensor 220.

In other embodiments, full sample tube sensor 220 may be a commercially-available “load” or “force” sensor operably coupled to packaging machine controller 2811. This may be accomplished using controls and feedback via packaging machine controller 2811 with a commercially available actuator or load/force cells in place of the springs 232 of in container holder 223. The load/force cell sensor would be disposed between the lower fixed plate 231 and upper floating plate 230 of the dual support plate assembly 233 which are in abutting contact with each other. Commercially-available thin film type sensors can be used for this application sandwiched between plates 231 and 230. When the actual load/force sensed during the sample tube 202 filling operation reaches a predetermined load/force setpoint preprogrammed into controller 2811, the controller determines the tube is filled to capacity by comparing the setpoint to the actual sensed load/force. The load/force sensing arrangement is clear to those skilled in the art based on the foregoing description without further undue elaboration or need for drawings.

It bears noting that after the piston-plunger 155 returns to its upward position, the dual support plate assembly 233 returns to the expanded condition as floating plate 230 returns upwards via the upwards biasing action of springs 232 acting on the floating plate as the springs expand.

### Control System

In one embodiment, the foregoing method or process for operating agricultural sample packaging system 100 may be controlled by a microprocessor controlled processing system including programmable local machine controller 2811 and/or main system controller 2820. Controller 2811 and/or controller 2820 is operably and communicably coupled and linked to all of the actuators, sensors, and other devices disclosed herein and programmable to execute suitable control logic/program instructions (e.g., software) to automatically control operation of the entire sample packaging system 100. In some embodiments, operation of the sample packing apparatus 110 may be initiated by one or more local actuators 2811a, 2811b (FIG. 22) which activates the controller 2811 and/or controller 2820 to start the sample packaging operations. These actuator switches are operably coupled to controller 2811 (which in turn is operably coupled to controller 2820 as further described below) and may be located anywhere on sample packaging apparatus 110.

FIG. 2 is a high-level system block diagram showing the control system 2800 including programmable processor-based machine controller 2811 and main system controller 2820 referenced herein. System controller 2820 may include one or more processors, non-transitory tangible computer readable medium, programmable input/output peripherals, and all other necessary electronic appurtenances normally associated with a fully functional processor-based controller. Control system 2800, including controller 2820, is operably and communicably linked to the different soil sample processing and analysis systems and devices described elsewhere herein via suitable wired or wireless communication links to control operation of those systems and devices in a fully integrated and sequenced manner.

Referring to FIG. 2, the control system 2800 including programmable main system controller 2820 and/or local machine controller 2811 may be mounted on a translatable self-propelled or pulled vehicle 2802 (e.g., tractor, trailer, combine harvester, truck, ATV, etc.) including those disclosed in U.S. Application Nos. 3/260772 filed on 31 Aug. 2021; 63/260,776 filed on 31 Aug. 2021; and 63/260,777 filed on 31 Aug. 2021. The vehicle may be the same vehicle which collects the agricultural samples such as soil samples. In other embodiments, the controller may be part of a stationary workstation or facility. The sampling vehicle 2802 and its boundaries are designated by dashed box in FIG. 2 (those items within the box being mounted onboard the sampling vehicle in the illustrated embodiment). The packaging apparatus 110 may be mounted on the same vehicle 2802 or a stationary workstation as the main system controller 2820, or be separate therefrom. Local machine controller 2811 is mounted on packaging apparatus 110.

Main control system 2800 generally includes programmable controller 2820, non-transitory tangible computer or machine accessible and readable medium such as memory 2805, and a network interface 2815. Computer or machine accessible and readable medium may include any suitable volatile memory and non-volatile memory or devices operably and communicably coupled to the processor(s). Any suitable combination and types of volatile or non-volatile memory may be used including as examples, without limitation, random access memory (RAM) and various types thereof, read-only memory (ROM) and various types thereof, hard disks, solid-state drives, flash memory, or other memory and devices which may be written to and/or read by the processor operably connected to the medium.

Both the volatile memory and the non-volatile memory may be used for storing the program instructions or software. In one embodiment, the computer or machine accessible and readable non-transitory medium (e.g., memory 2805) contains executable computer program instructions which when executed by the system controller 2820 cause the system to perform operations or methods of the present disclosure including measuring properties and testing of soil and vegetative samples. While the machine accessible and readable non-transitory medium (e.g., memory 2805) is shown in an exemplary embodiment to be a single medium, the term should be taken to include a single medium or multiple media (e.g., a centralized or distributed database, and/or associated caches and servers) that store the one or more sets of control logic or instructions. The term “machine accessible and readable non-transitory medium” shall also be taken to include any medium that is capable of storing, encoding or carrying a set of instructions for execution by the machine and that cause the machine to perform any one or more of the methodologies of the present disclosure. The term “machine accessible and readable non-transitory medium” shall accordingly also be taken to include, but not be limited to, solid-state memories, optical and magnetic media, and carrier wave signals.

Network interface 2815 may be configured to communicate with the soil or other bulk agricultural material collection system on the vehicle which is retrieving samples (e.g., soil, etc.) from the agricultural field, and sample post-packaging/containerizing systems such as a sample slurry preparation, processing, and chemical analysis systems and devices (collectively represented by box 2803 in FIG. 2).

The agricultural sample packaging system 100 machine network 2810 can include at least one local microprocessor-based machine controller 2811 and a plurality of different type sensors 2812. Sensors 2812 may be operably and communicably linked to local machine controller 2811 and optionally system controller 2820 through controller 2811; each controller being configured to receive and send data/signals from/to the sensors. In some embodiments, packaging apparatus 110 with local machine controller 2811 mounted thereto may be one vehicle which traverses the agricultural field along with the bulk sample collection system and main system controller 2820 may be located on a remote separate vehicle or in a stationary location.

The sensors 2812 may include full sample tube sensor 220 previously described herein, and other linear positional or status sensors 2812a integrated with cap actuator 160, compaction and sample transfer piston-plungers 150 and 155 respectively, sample blade mechanism actuator 135, and cleaning blade mechanism 140 actuator 145 to apprise the system controller 2820 of the position or status of the those devices (e.g., piston-plungers up or down, sample and cleaning blades mechanism inserted or withdrawn from die block 124, etc.). The status sensors may also include accelerometers to provide feedback to the system controller 2820 that a device of the packaging system physically moved in response to an action/motion initiated by a control signal from the controller (e.g. sample and cleaning blades mechanism inserted/withdrawn, piston-plungers up/down, etc.). Geolocation tracking sensors such as GPS (global positioning system) may also be included if the sample packaging system is mounted on a vehicle which travels across the agricultural field. Accordingly, the control system knows the operational status, position, and condition of each of at least the major components of the agricultural sample packaging system 100 under its control at any given moment. This information is used by the machine network controller 2811 and/or system controller 2820 to automatically control the entire agricultural sample packing operations of the packaging apparatus 110 via machine network 2810, and detect if an operational malfunction of packaging apparatus has occurred. This is particularly useful if the apparatus 110 is being controlled from a remote location via a communicably linked laptop, tablet, cell phone, etc. In addition, the GPS sensor 2854 communicably linked to the packaging machine network 2810 as seen in FIG. 2 permits the machine and/or system controllers 2811, 2820 to pinpoint where in the agricultural field the soil sample was collected if the sample collection system equipment is used alongside the packaging apparatus 110 when the sample is collected and then packaged. The RFID tag associated with each packaged sample permits the associated GPS geolocation information to be tracked for each sample.

The local machine controller 2811 which may be mounted onboard packaging apparatus 110 controls operation of the agricultural sample packaging system 100 in cooperation with system controller 2820 in one embodiment. In other embodiments, machine controller 2811 may control operation of the packaging apparatus 110 alone via preprogrammed control logic/instructions if the controller is not linked to a main system controller 2820, or still be communicably coupled to the main system controller for data/information exchange and programming, but not for purposes of direct control of the sample packaging system components.

Local machine controller 2811 includes all of the usual appurtenances and auxiliary electronic devices similar to main system controller 2820 (e.g., memory, power supply, etc.) for forming a normal fully functional microprocessor-based control system configured to control operation of packaging apparatus 110.

With continuing reference to FIG. 2, the RFID scanner or reader 2850 previously described herein which is mounted on or may be nearby packaging apparatus 110 is operably and communicably coupled to packing system machine network 2810 via communication link 2852. Communication link 2852 may be wired or wireless. The unique RFID tag 2850a associated with each collected and packaged agricultural sample in sample tube 202 may be automatically scanned and read in one embodiment when end cap 204a which contains the tag is placed on the reader at the start of the sample tube filling operation. The tag may be read in some embodiments when the Fill Tube actuator 2811a is manually activated (e.g., pushed or thrown) by the packaging apparatus operator. This ensures that the correct geolocation is associated with the sample as the operator and vehicle should still be in the same physical location where the soil sample was collected from the agricultural field at that time. As previously described herein, the RFID tag may alternatively be located on the cylindrical body of the sample tube 202 or slideable plunger cap 204b inside the tube and read by controller 2811 of the machine network 2810. The unique sample ID information is transmitted to packaging system machine controller 2811, which may in turn may share that information with the main system controller 2820. The unique RFID tag associated with each sample tube 202 and its sample contents allows the sample to be tracked from initial packaging, other staging and processing of the sample pending chemical system, and finally chemical analysis. With use of the GPS information collected for with each sample that identifies the exact location in the agricultural field where the sample was collected, the chemical analysis results of the analytes of interest may be readily correlated back to a particular location or region in the field to determine the soil amendments necessary there.

The packaging system 100 may be locally controlled by machine controller 2811, which in turn is controlled and programmed by an personal electronic device (PED) 2851 with onboard microprocessor, memory, power supply, and all other usual auxiliary device and components associated with such devices. Such personal electronic devices 2851 may include for example without limitation a tablet, laptop, notebook, cell phone, and other similar devices located onboard vehicle 2802. Device 2851 acts as a user interface and input device which initiates automated operation of the agricultural sample packaging system 100 and packaging apparatus 110 via machine controller 2811. Personal electronic device 2851 may have a graphic user interface such as a touchscreen for such a purpose. Personal electronic device 2851 is operably and communicably coupled to packing system machine network 2810 via communication link 2853, which may be wired or wireless.

It bears noting that in embodiments where the entire agricultural sampling collection, packaging, and chemical analysis systems are mounted on a single field vehicle 2802 for in-situ analysis of the samples, the agricultural sample packaging system 100 may be controlled by the main system controller 2820 in lieu of a separate machine controller 2811. In such a case, the array of packaging system sensors 2812 may communicate directly with system controller 2820.

The network interface 2815 can be configured for wired and/or wireless bidirectional communications which may include at least one of a GPS transceiver, a WLAN transceiver (e.g., WiFi), an infrared transceiver, a Bluetooth transceiver, Ethernet, Near Field Communications, or other suitable communication interfaces and protocols for communications with the other devices and systems including the agricultural sample packaging system 100. The network interface 2815 may be integrated with the control system 2800 as illustrated in FIG. 2, the machine network 2810, or elsewhere. The I/O (input/output) ports 2829 of control system 2800 (e.g., diagnostic/on board diagnostic (OBD) port) enable communication with another data processing system or device (e.g., display devices, sensors, etc.).

The programmable controller 2820 may include one or more microprocessors, processors, a system on a chip (integrated circuit), one or more microcontrollers, or combinations thereof. The processing system includes processing logic 2826 for executing software instructions of one or more programs and a communication module or unit 2828 (e.g., transmitter, transceiver) for transmitting to and receiving communications from the machine network 2810 of sampling machine or vehicle 2802 via direct communication link 2831 or network interface 2815. The communication unit 2828 may be integrated with the control system 2800 (e.g. controller 2820) or be separate from the controller. In one embodiment, the communication unit 2828 may be in operable data communication with the machine/vehicle network 2810 via a diagnostic/OBD port of the I/O ports 2829.

Programmable processing logic or instructions 2826 of the control system 2800 which directs the operation of system controller 2820 including one or more processors may process the communications (i.e. data/information) received via the communication unit 2828 or network interface 2815 from the agricultural sample packaging system 100 including without limitation sensor associated with the status and operation of the packaging apparatus 110 and components thereof under the control of programmable system controller 2820. The memory 2805 of control system 2800 is configured for preprogrammed variable or setpoint/baseline values, storing collected data, and computer instructions or programs for execution (e.g. software 2806) used to control operation of the controller 2820, which in turn controls operation of packaging apparatus 110 and sample processing/analysis devices 2803. The memory 2805 can store, for example, software components such as testing software for analysis of soil and vegetation samples for performing operations of the present disclosure, or any other software application or module, images 2808 (e.g., captured images of crops), alerts, maps, etc. The system 2800 can also include an audio input/output subsystem (not shown) which may include a microphone and a speaker for, for example, receiving and sending voice commands or for user authentication or authorization (e.g., biometrics).

In some embodiments of agriculture sample packaging system 100 can further preferably include a sensing system 2812 comprising a plurality or array of different type sensors useful and associated with packaging and tracking the soil sample. The sensing system and its sensors are in data and control communication with packaging system machine controller 2811 and/or main system controller 2820. Other sensors which communicate with system controller 2820 may be associated with operation of the sample collection apparatus 8002 and components thereof including various equipment positional or orientation sensors, proximity sensors, etc. The agricultural material sample packaging system in combination with sensing system can provide complete automated control of the sample collection apparatus 8002 via the packaging system machine controller 2811 and/or main system controller 2820.

The main system controller 2820 communicates bi-directionally with memory 2805 via communication link 2830, machine or sample collection system network 2810 directly via communication link 2831 and/or alternatively via communication link 2837 associated with network interface 2815, the network interface 2815 via communication link 2832, display device 2830 and optionally a second display device 2825 via communication links 2834, 2835, and I/O ports 2829 via communication links 2836. System controller 2820 further communicates with the soil sample processing and analysis systems and devices 2803 via the wired/wireless communication links 5752 previously described herein via the network interface 2815 and/or directly as shown.

Display devices 2825 and 2830 can provide visual user interfaces for a user or human operator. The operator may be located onboard the mobile vehicle in one embodiment which traverses the agricultural field or at a remote operating position or station distal from the packaging apparatus 110. The display devices may include display controllers with onboard programmable microprocessors. In some embodiments, the computerized display device 2825 may therefore be a portable tablet device, cell phone, laptop, notebook, or other processor-based computing device with a touchscreen and/or keyboard (software based or physical hardware) that acts as an input/output device and which displays data (e.g., equipment status and position, and other relevant operational and maintenance information) and communicates with controller 2820. The computerized display device 2825 therefore receives input from the user or operator for controlling packaging apparatus 110.

The agricultural sample packaging system 100 disclosed herein is usable with and may form part of an overall agricultural sampling and analysis systems such as those described in U.S. Patent Application Publication No. 2018/0124992A1 and PCT Publication No. WO2020/012369, and other systems are described in U.S. Application Nos. 62/983,237, filed on 28 Feb. 2020; 63/017,789, filed on 30 Apr. 2020; 63/017,840, filed on 30 Apr. 2020; 63/018,120, filed on 30 Apr. 2020; 63/018,153, filed on 30 Apr. 2020; 63/191,159, filed on 20 May 2021; 63/191,166, filed on 20 May 2021; 63/191,172, filed on 20 May 2021; Ser. No. 17/326,050, filed on 20 May 2021; 63/191,186, filed on 20 May 2021; 63/191,189, filed on 20 May 2021; 63/191,195, filed on 20 May 2021; 63/191,199, filed on 20 May 2021; 63/191,204, filed on 20 May 2021; Ser. No. 17/343,434, filed on 9 Jun. 2021; 63/208,865, filed on 9 Jun. 2021; Ser. No. 17/343,536, filed on 9 Jun. 2021: 63/213,319, filed on 22 Jun. 2021; 63/260,772 filed on 31 Aug. 2021; 63/260,776 filed on 31 Aug. 2021; and 63/260,777 filed on 31 Aug. 2021, and PCT Application Nos. PCT/IB2021/051076, filed on 10 Feb. 2021; PCT/IB2021/051077, filed on 10 Feb. 2021; PCT/IB2021/052872, filed on 7 Apr. 2021; PCT/IB2021/052874, filed on 7 Apr. 2021; PCT/IB2021/052875, filed on 7 Apr. 2021; and PCT/IB2021/052876, filed on 7 Apr. 2021.

### Examples—Sample Packaging Apparatus

The following are non-limiting examples of the sample packaging apparatus.

Example 1—an agricultural sample packaging apparatus comprising: a bulk material chamber configured for receiving a sample; a sample collection chamber; a die block disposed between the bulk material and sample collection chambers, the die block including a plurality of through die slots in communication with the bulk material and sample collection chambers; a sample blade mechanism comprising a plurality of elongated sample blades movably insertable through the bulk material chamber and die slots from a first side of the die block, wherein the sample blades are operable to extrude the sample from the bulk material chamber through the die slots and into the sample collection chamber.

Example 2—the apparatus according to Example 1, wherein the apparatus comprises a rotatable carousel including a container holder configured to removably hold a sample container.

Example 3—the apparatus according to Example 2, wherein the carousel is rotatable between an inward closed position in which the sample container is positioned beneath the sample collection chamber and an outward open position in which the sample container is not beneath the sample collection chamber.

Example 4—the apparatus according to any one of Examples 2 to 3, wherein the apparatus comprises a cleaning blade mechanism comprising a plurality of elongated cleaning blades movably insertable through the sample collection chamber and die slots from a second side of the die block.

Example 5—the apparatus according to any one of Examples 2 to 4, further comprising a compaction piston-plunger configured to compress the sample in the bulk material chamber.

Example 6—the apparatus according to Example 5, further comprising a sample transfer piston-plunger configuration to force the sample downwards and outwards from the sample collection chamber.

Example 7—the apparatus according to any one of Examples 2 to 6, further comprising a programmable controller configured to control operation of the sample packaging apparatus.

Example 8—the apparatus according to any one of Examples 2 to 7, wherein the sample is soil.

Example 9—the apparatus according to any one of Examples 2 to 8, wherein the apparatus comprises a support frame configured for mounting to a mobile vehicle.

Example 10—the apparatus according to Example 1, wherein each of the sample blades comprises a compression spring.

Example 11—the apparatus according to Example 10, wherein each sample blade comprises a bifurcated end comprising a pair of the springs which act on a pair of spaced apart spring plates coupled to each sample blade.

Example 12—a method for packaging an agricultural sample comprising: adding a sample to a bulk material chamber; inserting a plurality of sample blades through the bulk material chamber; forcing the sample through a die block with the sample blades forming sample material plugs; and collecting the sample material plugs in a sample collection chamber.

Example 13—the method according to Example 12, further comprising before the insertion step, a step of rotating a carousel holding a sample container and positioning the sample container beneath the sample collection chamber.

Example 14—the method according to Example 13, further comprising depositing the sample material plugs in the sample container.

### Sample Unloading System

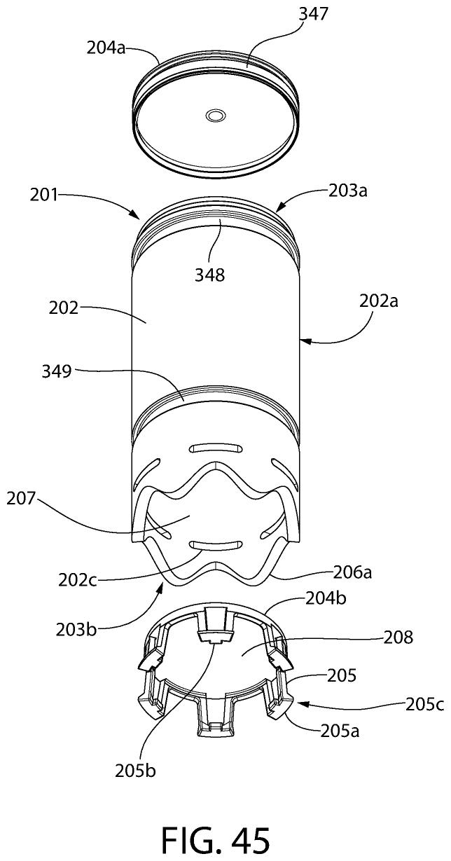

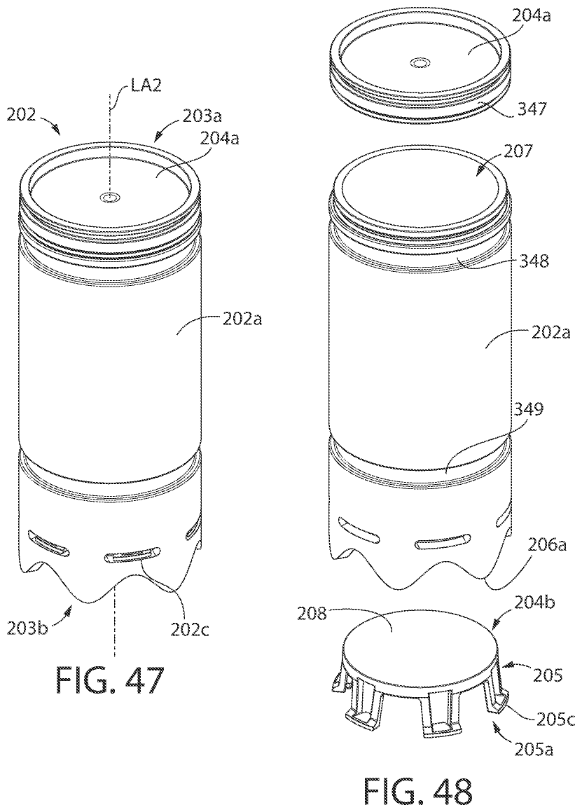

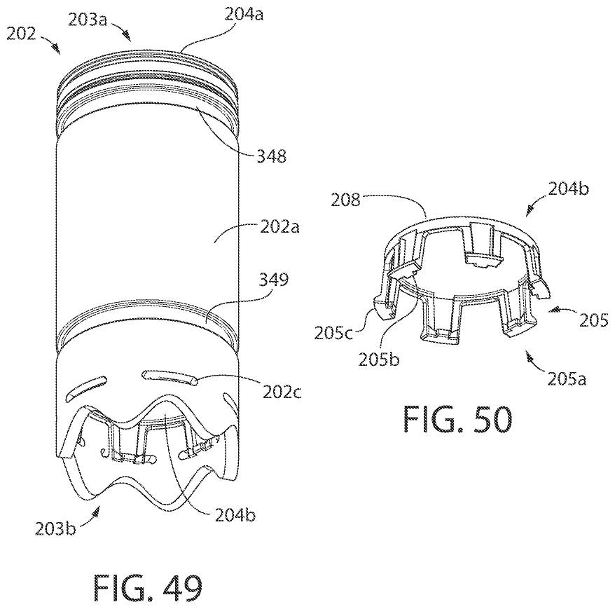

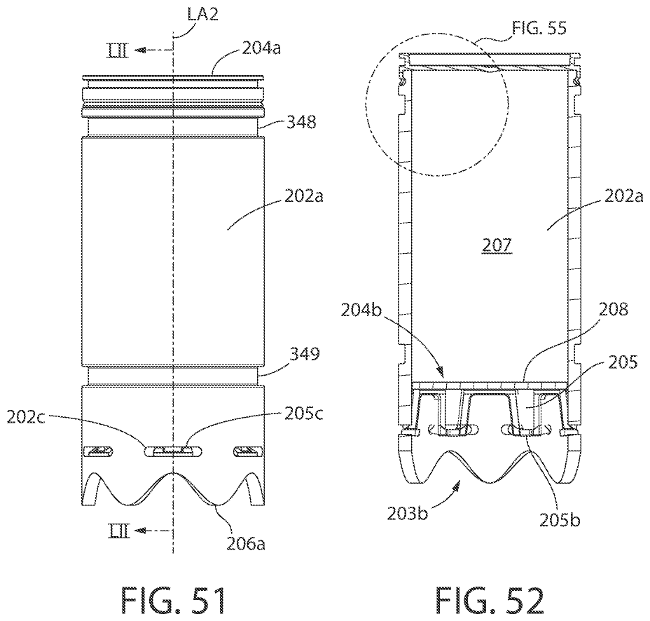

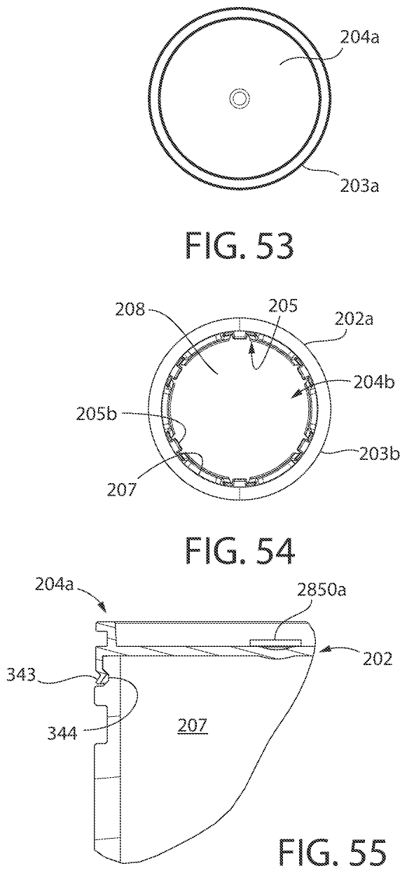

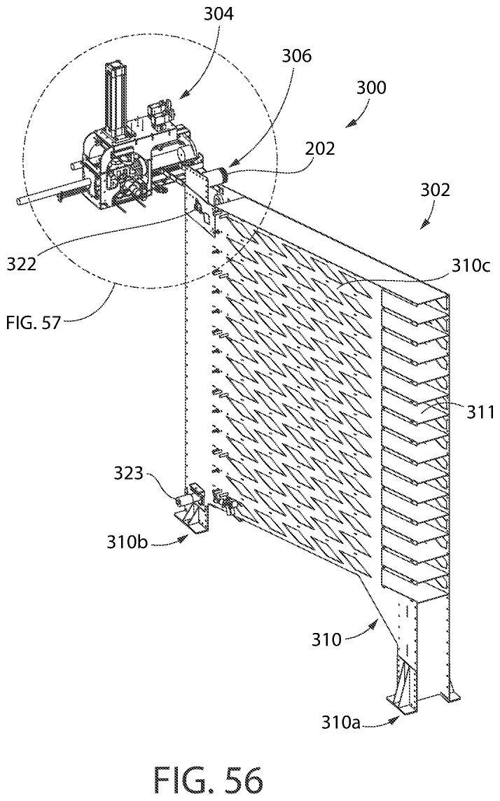

FIGS. 45 and 47-110 depict one non-limiting embodiment of an agricultural sample handling system and various parts thereof comprising an unloading system 300 according to the present disclosure. General reference is intended to these figures below. Specific figures may be called out where appropriate which illustrate specific aspects or features of the unloading system being discussed.

The unloading system 300 is configured and operable to both stage the sample containers 201 in a sequenced manner such as sample tubes 202 packed with the collected agricultural samples (e.g. soil, plant, etc.) by sample packaging system 100 as described above, and then unload the samples from the tubes for further processing and eventual chemical analysis.

System 300 generally includes a sample staging rack 302 and unloading apparatus 304 operably coupled to the rack. Sample transfer mechanism 306 is configured and operable to transfer the sample container 201 such as sample tube 202 from the staging rack to the unloading apparatus. The transfer mechanism may load a single tube at a time into the unloading apparatus 304 in a serial manner for unloading in one non-limiting embodiment. Staging rack 302 and unloading apparatus 304 are physically closely coupled and adjacent each other in a preferred but non-limiting embodiment. In some embodiments, the rack may be physically coupled to the unloading apparatus, but in other embodiments may be simply located adjacent thereto. Each of these features and aspects of system 300 and operation to stage and remove the samples from the sample tubes 202 will now be described in further detail below.

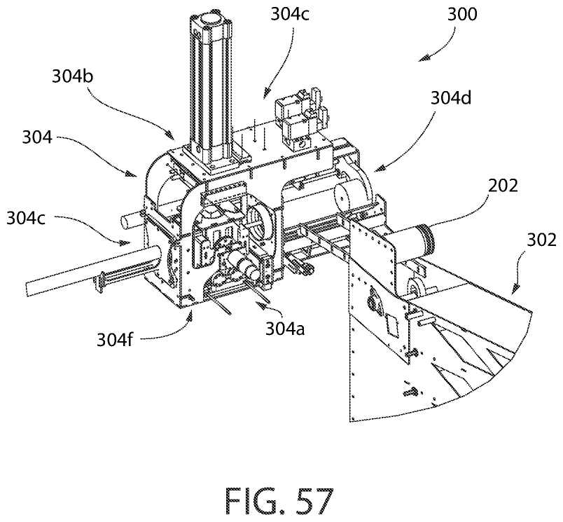

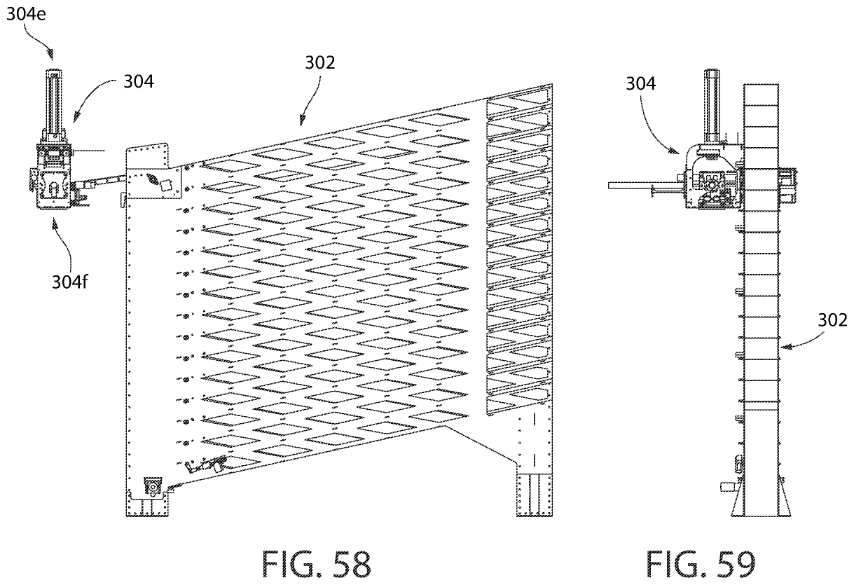

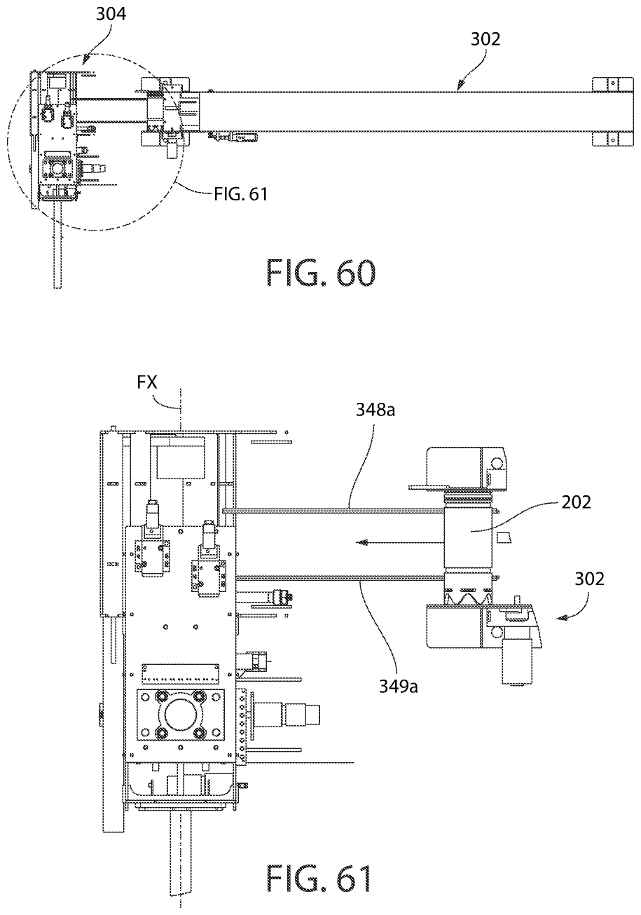

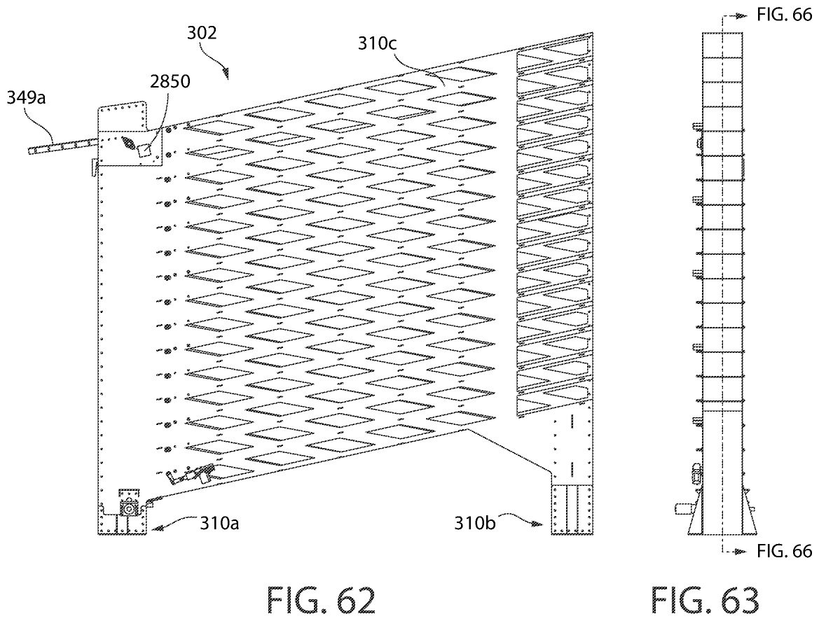

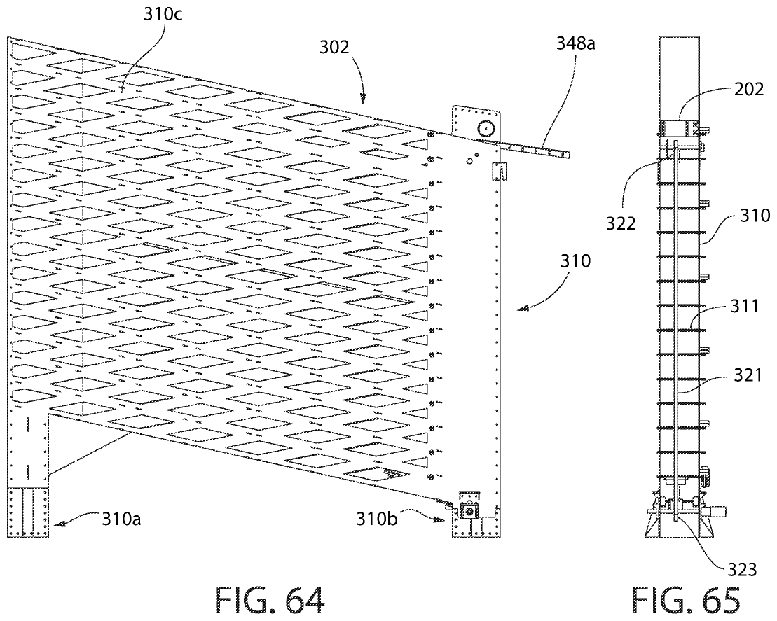

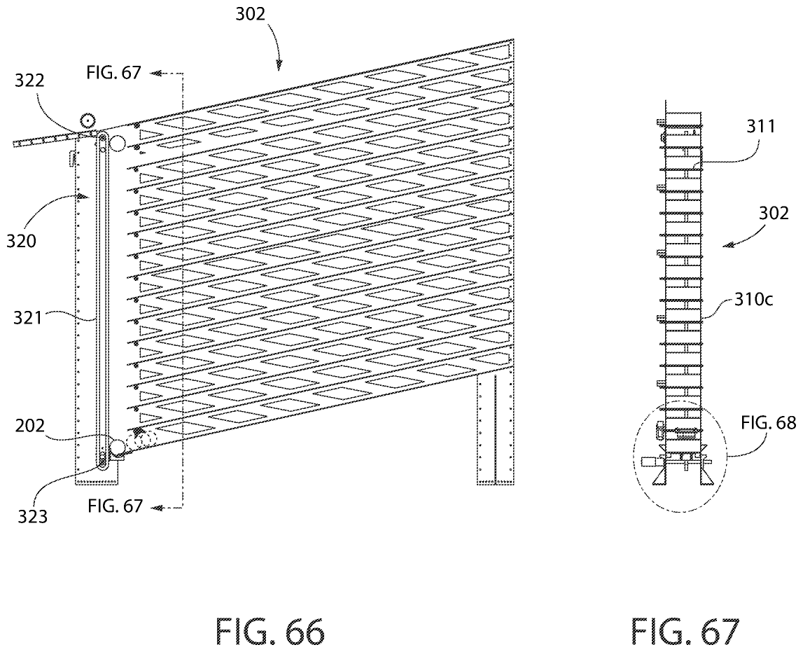

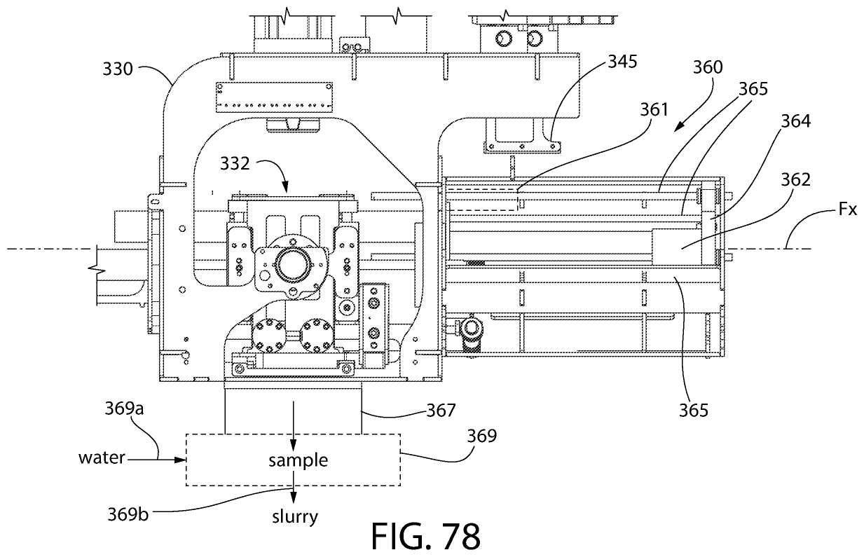

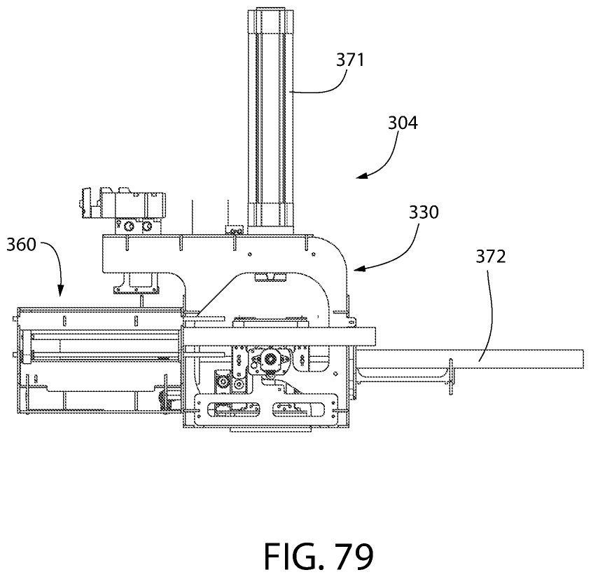

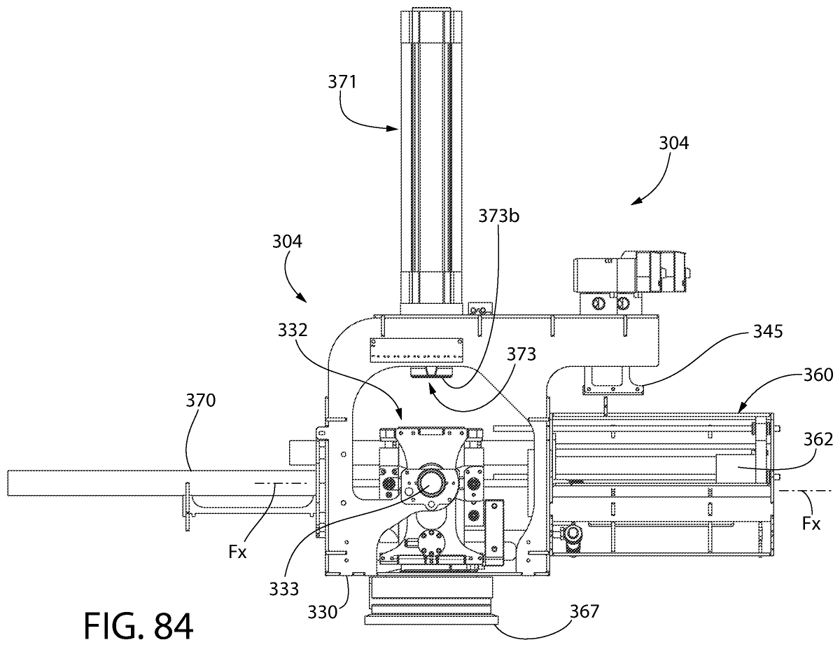

For convenience of description and directional reference only, unloading apparatus 304 may be considered to define a front 304a, rear 304b, left lateral side 304c, and right lateral side 304d in top plan view (see, e.g., FIG. 57). Apparatus 304 may also be considered to define a top 304e and bottom 304f. A longitudinally-extending sample container feed axis FX is defined extending through lateral sides 304c, 304d (see, e.g., FIGS. 78 and 84). Transverse directions and orientations are defined herein as being disposed at a perpendicular 90 degree or acute angles to axis FX.

Sample staging rack 302 comprises a support frame 310 configured to rest on a generally horizontal support surface which may be a stationary floor, flat bed of a wheeled vehicle such as a truck, trailer, agricultural implement, or any other suitable type surface to which the rack may be rigidly mounted in a stable manner. The frame in some embodiments may therefore include a pair of distal and proximal feet 310a, 310b configured for seating on the support surface. The terms “distal” is defined herein as being farthest from unloading apparatus 304 and “proximal” is defined as closest thereto. The frame 310 includes a side panel plate 310c on each side which at least partially encloses the feed ramps 311 to prevent the sample tubes 202 from sliding out of the staging rack 302. Plates 310c may include plural openings to allow the user to view the sample tubes 202 on the staging rack feed ramps. Frame 310 in various embodiments may be formed of an assemblage of various types and shapes of suitable metal structural members connected together by any means to form a self-supporting support frame. Any suitable shape and construction of the frame may be used. Staging rack 302 is horizontally elongated and may be oriented perpendicularly to sample unloading apparatus 304 and container feed axis FX.

Frame 310 supports at least one inclined feed ramp 311 configured and having an elongated horizontal length for receiving and staging one or more sample tubes 202. In a preferred but non-limiting embodiment, a plurality of feed ramps are provided in vertically stacked and parallel relationship. The ramps 311 each hold a plurality of the elongated tubes 202, which may be cylindrical, in a horizontally abutting and crosswise manner oriented transversely to the length of the ramps. The cylindrical walls of tubes are therefore in abutting contact in sidewall to sidewall relationship. The ramps are vertically spaced apart by a distance which can accommodate the diameter of the sample tubes 202 while allowing them to roll freely.

Feed ramps 311 of staging rack 302 slope downwards at a suitable angle towards unloading apparatus 304 so that the sample tubes 202 may automatically and freely roll towards the unloading apparatus via gravity. The sample tubes therefore roll from a distal ends 312 of the rack and feed ramps towards a proximal end 313 of the rack adjacent to unloading apparatus 304.

In one embodiment, the staging rack 302 is coupled to the unloading apparatus 304 by a pair of spaced apart track rails 348a, 349a spanning between the rack and tube loading mechanism 306 of the apparatus. The rails rollingly engage a pair of circumferential grooves 348, 349 formed on the exterior surface of the cylindrical wall 202a of sample tube 202 proximate to each end to guide the tube onto the tube feed chute 363 of the tube loading mechanism 360 of the unloading apparatus.

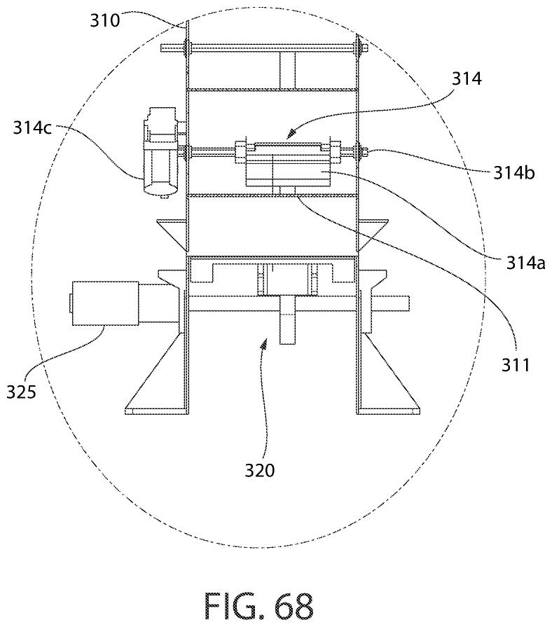

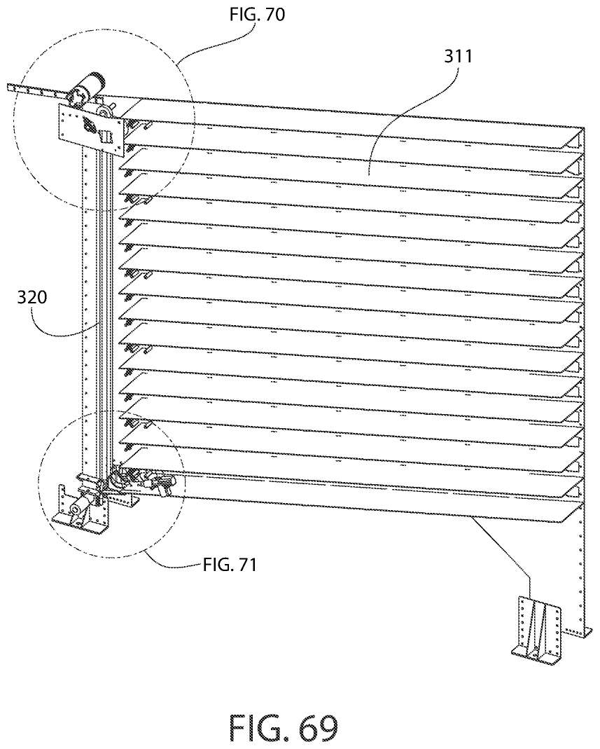

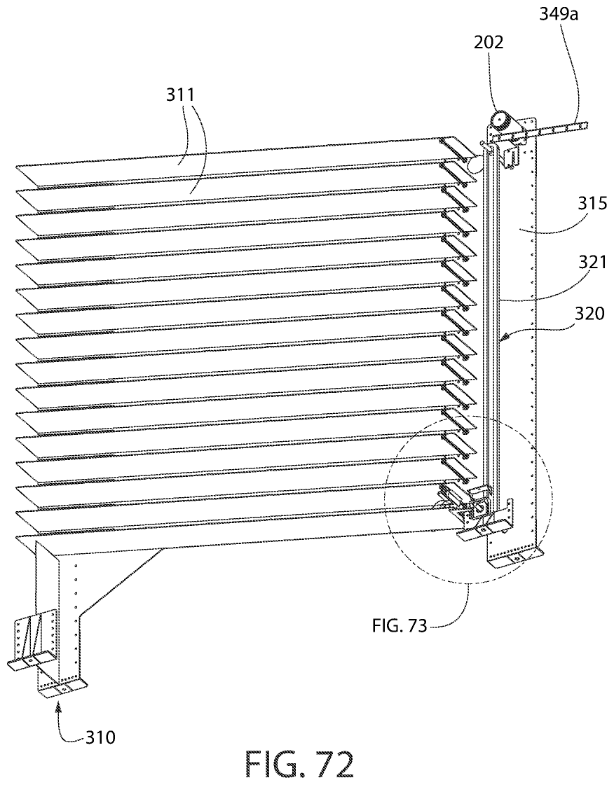

Referring to FIGS. 68 and 71-73, sample transfer mechanism 306 in one aspect includes a rotary paddle feed gate 314 positioned at the proximal end of each feed ramp 311. The feed gates stop the leading sample tubes 202 from rolling off staging rack 302 into a vertically-extending passageway 315 at the proximal end 313 of rack 302 between the rack and unloading apparatus 304. Gates 314 are configured and operable to provide sequenced feeding of the sample tubes into unloading apparatus 304 according to a preprogrammed tube feed sequence in system controller 2820, as further described herein.

Paddles feed gates 314 are each horizontally elongated in the side-to-side rear direction of the unloading apparatus 304 and positioned to engage the leading sample tube 202 on each feed ramp 311 of the staging rack 302 (i.e. tube closest on ramp to unloading apparatus 304). A feed gate 314 is positioned above the proximal ends of each feed ramp 311 just beneath the next feed ramp above in the staging rack such that the sample tube 202 passes beneath the gate when fed and admitted to the transfer mechanism.

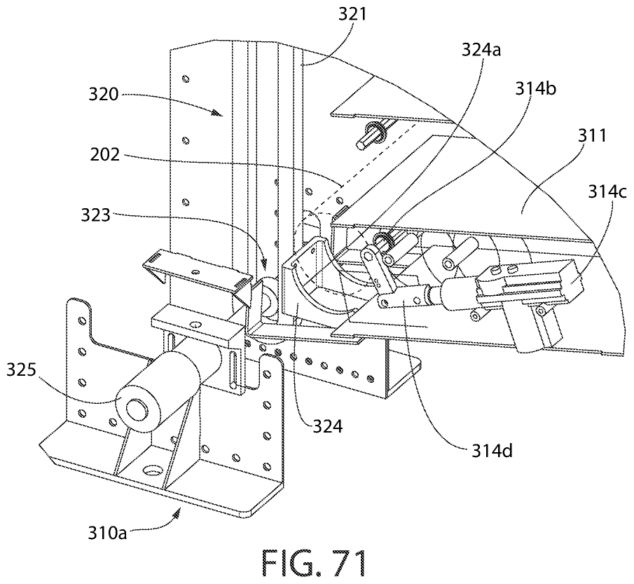

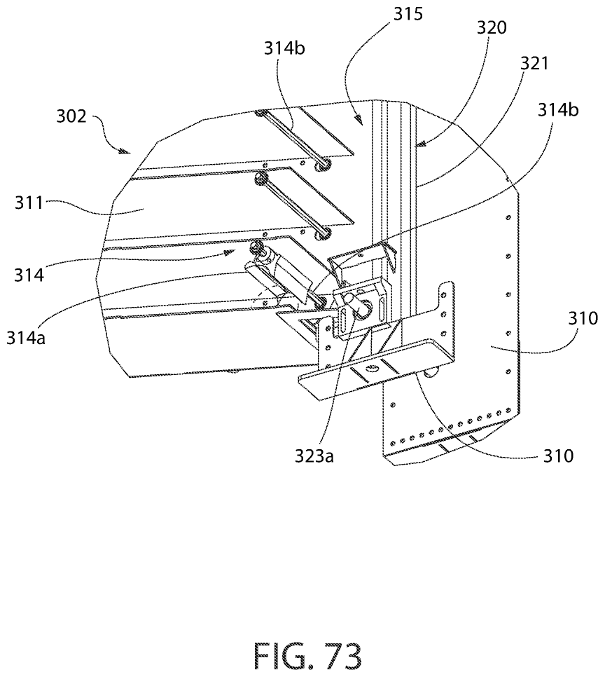

In one embodiment, the feed gates 314 each include a plurality of radial blades 314a rotatable about the transverse pivot axis of the gate defined by a transverse pivot pin 314b mounted to frame 310 of staging rack 302 at each side of the feed ramp (see, e.g., FIG. 73). In one embodiment, each feed gate 314 comprises a pair of radial blades disposed at an acute angle to each other and spaced angularly apart to engage and receive the sidewalls of a sample tube 202 (note FIG. 73 shows only a single feed gate on one feed ramp 311 to avoid clutter in the figure—other pivot pins 314b however visible on ramps above). The feed gates 314 are each pivoted back and forth in a toggle action to pass the leading sample tube 202 to the transfer mechanism 306 by a separate dedicated actuator 314c (best shown in FIG. 71), while blocking the second leading sample tube on the feed ramp. One actuator 314c may be coupled to the pivot pin 314b of each gate, such as in one non-limiting embodiment by an L-shaped angled link arm 314d (best shown in FIG. 71) configured to produce the desired toggle action of the gate. Link arm 314d has a proximal pinned pivot joint between each leg of the arm to allow the arm legs to rotate relative to each other for turning the pivot pin 314b of the feed gate 314. Actuator 314c may be any suitable commercially-available linear electric actuator, pneumatic actuator, or hydraulic actuator configured and operable to actuate link arm 314d and pivot the feed gate. The feed gate operates to engage, pivot, and push a lead sample tube forward to the proximal end of the feed ramp 311 to a position where it can be engaged and loaded into the unloading apparatus 304 by the transfer mechanism 306, as further described herein.

The sample transfer mechanism 306 in another aspect further comprises a tube elevator 320 configured and operable to engage and extract a sample tube 202 dispensed from the active feed gate 314 of staging rack 302. In one non-limiting embodiment, the tube elevator 320 may comprise a chain drive including a circulating continuous-loop chain 321, toothed upper sprocket 322, toothed lower sprocket 323, and forked lifting member 324 fixedly coupled to and movable vertically with the chain. The chain may be of a type generally similar to a bicycle chain for comparison; however, other types of chains may be used. Lifting member 324 may be somewhat L-shaped in one embodiment and configured to engage, lift, and remove the sample tube 202 from the rotary paddle feed gate 314, and load the sample tube into the unloading apparatus 304 from the staging rack 302. The lifting member in one non-limiting embodiment may have a pair of outwardly projecting prongs 324a configured to engage and extract the tubes 202 from the rack. Prongs 324a may be arcuately curved in one embodiment with a radiused engagement surfaces 324a coinciding to the radius of the sample tube 202 to snugly engage the wall 202a of the tube.

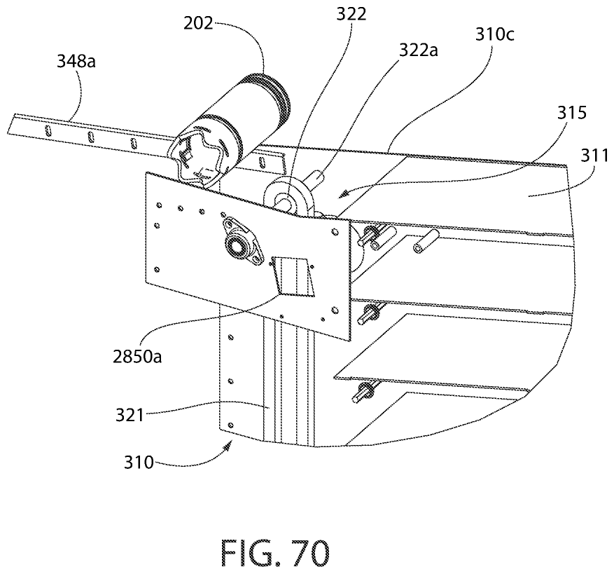

The tube elevator 320 is located in vertically-extending passageway 315 between staging rack 302 and unloading apparatus 304. An electric motorized chain drive 325 coupled to either one of the shafts 322a or 323a of the upper sprocket 322 or lower sprocket 323 respectively rotates the chain between the sprockets such that lifting member 324 moves vertically in the passageway past the proximal ends of each feed ramp 311. Lifting member 324 receives and grasps a sample tube 202 from the feed ramp when presented for loading into apparatus 304 by operation of one of the rotary paddle feed gates 314. Chain 321 is vertically oriented and elongated as shown. Each sprocket shaft 322a, 323a is rotatably supported at its ends between opposing the sides of the frame 310, such for example as between side panel plates 310c (see, e.g., FIG. 70).

In operation, the segment or side of the chain loop closest to the ends of the feed ramps 311 moves upwards past the feed gates 314, and the segment or side of the loop closest to unloading apparatus 304 concomitantly moves downwards as the chain rotates. Lifting member 324 rising with the upward moving segment or side of the chain plucks a sample tube 202 passed on by one of the rotary paddle feed gates 314 presented for loading and carries the tube up with the chain to the top. As previously described herein, the feed gate 314 has a pivotable toggle action such that the gate pivots back and forth to pass the leading sample tube through the gate for engagement by the lifting member 324, while at the same time engages the next tube in line on the feed ramp 311 to hold it on the ramp. Other arrangements are possible.

When the lifting member 324 rotates over the upper sprocket 322 at top of the tube elevator towards the unloading apparatus 304, the sample tube is tossed onto guide rails 348a, 349a previously described herein by the lifting member when it reverses direction with the downward moving side of the chain loop. The sample tube 202 is guided into unloading apparatus 304 by the rails 348a, 349a which engage circumferential grooves 348, 349 in the tube, respectively. The intermeshing rail and groove arrangement keeps the tube in line as it rolls into the unloading apparatus. The lifting member 324 on the chain loop will pass beneath the lower sprocket 323 and then reverse direction vertically to begin travel back upwards with the chain 321 to extract the next sample tube presented to the tube elevator 320. It bears noting that the chain 321 travels in the same direction during the tube loading/feeding process in a loop. In some embodiments, the tube transfer unloading operation into unloading apparatus 304 and sample tube feed may be timed by preprogrammed steps executed by system controller 2820 so that the tube elevator circulates continuously or intermittently, thereby advantageously allowing tubes to be staged for feed into the unloading apparatus while a tube is being unloaded in the unloading apparatus without any appreciable gap in time. This efficiently processes the sample tubes for unloading in the most optimum time expedient manner possible.

Although a chain elevator has been described and shown for tube elevator 320, the elevator may be embodied in other types of devices that perform the same function. For example, some embodiments of tube elevator 320 may comprise a belt type system comprising a circulator belt to which lifting member 324 is coupled and pulleys in place of the upper and lower sprockets. Other means of feeding sample tubes into unloading apparatus 304 for unloading which do not involve rotating belts or chains may be used. Accordingly, the invention is not limited to chain or belt drives alone.

Staging rack 302 further includes a sample tube tracking system comprising a plurality of tracking tag readers, such as RFID reader 2850 in one non-limiting embodiment. One RFID reader may be located at the proximal end of each feed ramp 311 and mounted to frame 310. A window 2850a may be provided in the frame at the end of each feed ramp so that the reader is exposed to the sample tube 202 on the ramp to read the tag. In other embodiments, the tracking tag may be a bar code readable by a visual barcode scanner in lieu of an RFID reader. Other forms of readable tracking tags and corresponding tag reading systems be they electronic or visual in nature may be used.

Sample unloading apparatus 304 will now be described in further detail. With continuing general reference to FIGS. 45 and 47-110, apparatus 304 comprises a support housing 330 configured to mount the apparatus to any suitable structure which may be a floor, vehicle such as a truck, trailer, agricultural implement, or any other suitable structure to provide a rigid and stable mounting. Any suitable shape and construction of apparatus housing may be used.

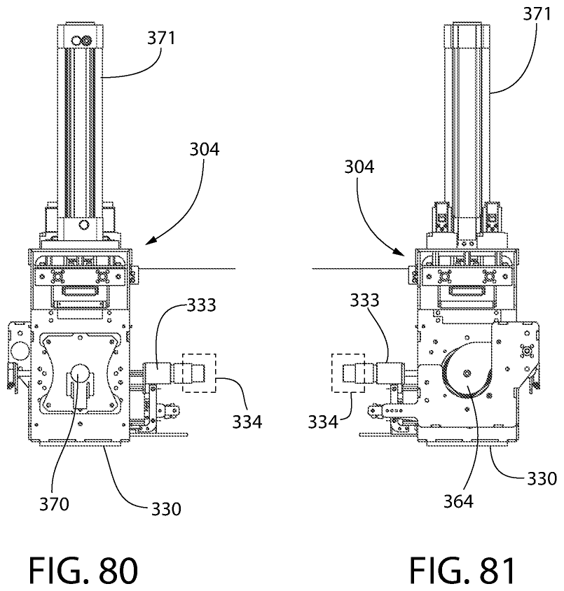

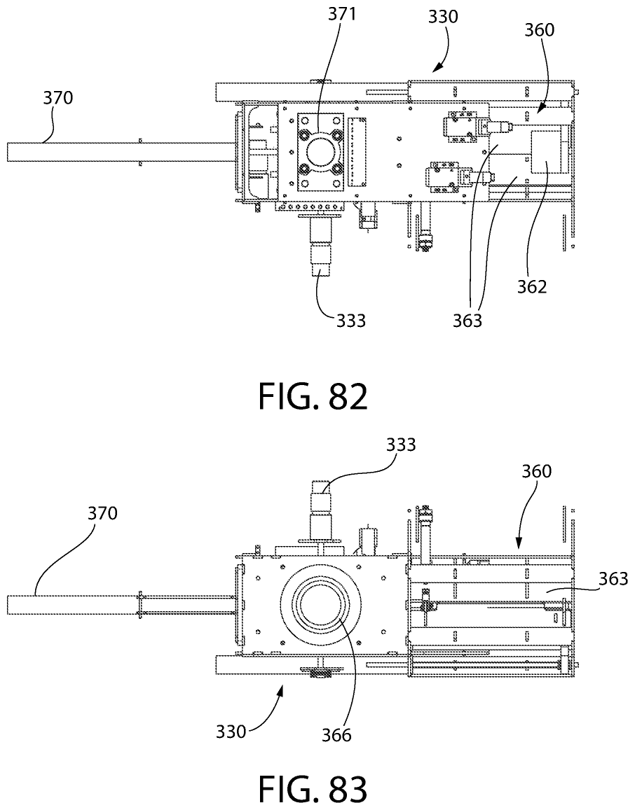

Unloading apparatus 304 further comprises a trunnion mechanism 329 rotatably supported by housing 330. In one non-limiting embodiment, the trunnion mechanism may include a rotatable and slideable container/tube carriage 332 configured to hold the sample tube 202. Carriage 332 is fixedly coupled to crosswise trunnion drive shaft 333 rotatably supported by the housing. Shaft 333 is oriented and elongated in the front to rear direction of the unloading apparatus perpendicularly to the container/tube feed axis FX. Carriage 332 is rotatable with shaft 333 in opposing rotational directions. In one implementation, the tube carriage can be rotated 360 degrees in either direction by trunnion drive motor 334 (shown schematically in dashed lines FIGS. 80-81) coupled to drive shaft 333.

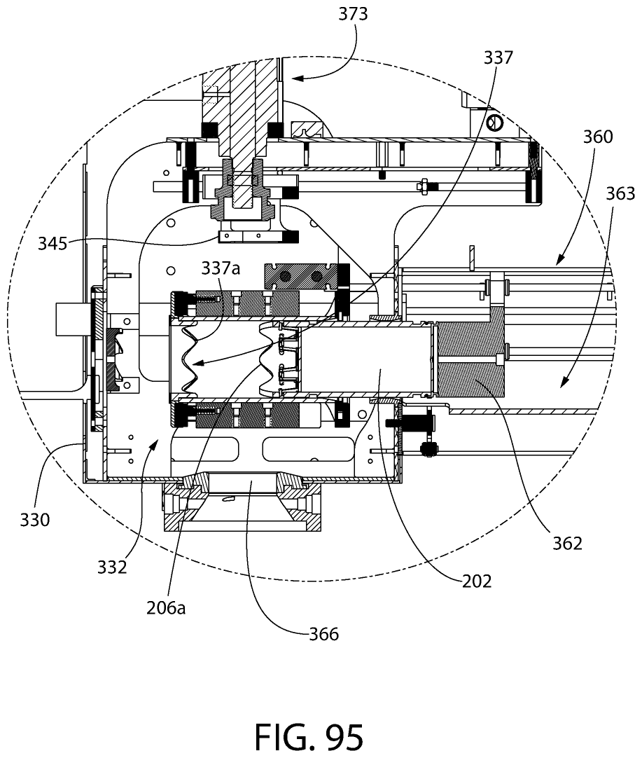

The rotatable carriage 332 further comprises a container receptacle 336 defining an elongated interior space configured for receiving, maneuvering, and supporting the sample tube 202 in a secure manner during the sample unloading process. Carriage 332 is operable to rotate the sample tube between an upright vertical position and an inverted vertical position via drive motor 334. Receptacle 336 includes an annular castellated support ring 337 at the bottom 338 and an open top 339 through which the sample tube can be inserted. Support ring 337 has an open center to allow the tube ejector piston-plunger 372 to enter the receptacle to access and engage the sample tube for ejecting the tube from the carriage 332. Support ring 337 defines an undulating surface which is complementary configured to mesh with the undulating castellated bottom end 203b of sample tube 202. Anti-rotation protrusions 206a on the tube lockingly engage the mating anti-rotation protrusions 337a formed by the castellated support surface 337 in the receptacle to rotationally lock the tube relative to carriage 332 (see, e.g., FIG. 95). This ensures that free terminal ends 205a of the retention protrusions 205 on slideable push cap 204b remain rotationally clocked or timed to engage their corresponding elongated retention slots 202c in the cylindrical walls of sample tube 202 (previously described herein) when the cap slides inside tube 202 and then re-engages the slots, as further described herein. The peaks and valleys of castellated support surface 337 and bottom end 203b of sample tube 202 may be arcuately curved or rounded. This advantageously guides the mating castellated surfaces together if the peaks and valleys of each are not rotationally aligned when first meshed together.

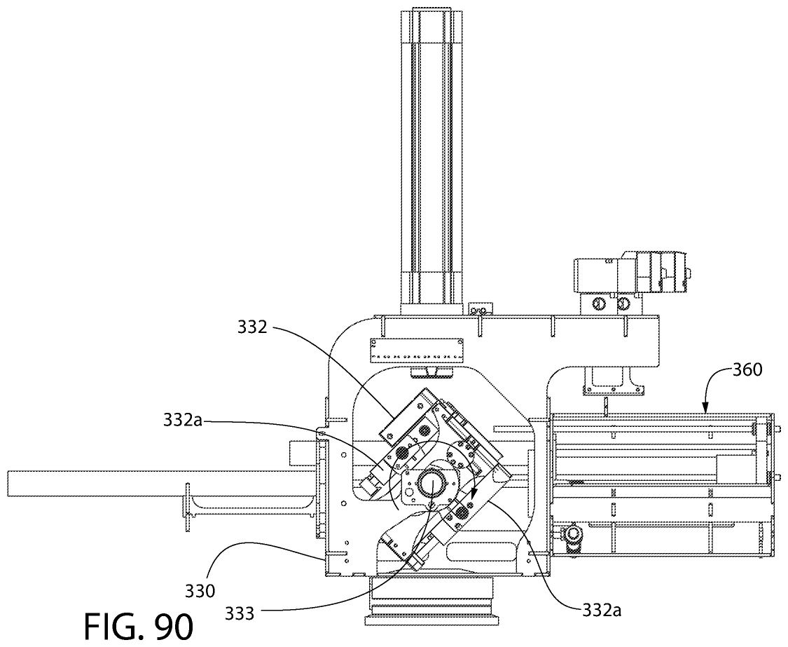

Carriage 332 further includes a pair of linear actuators 332a configured to slideably move the carriage linearly in two opposing directions within housing 330 between inward and outward positions (see, e.g., FIGS. 90-92). Actuator 337 may be any suitable commercially-available linear electric actuators, pneumatic actuators, or hydraulic actuators. In one embodiment, the actuators 332a may comprise a pair of pneumatic cylinder actuators; one actuator being coupled to each of two opposite sides of the carriage. It bears noting that the cylinder actuators are therefore rotatable with the carriage 332 during the sample unloading process. The cylinder actuators are used to uncap the sample tube 202 during the sample unloading process, as further described herein.

Unloading apparatus 304 further includes a movable closure plate 350 configured and operable to temporarily cover and close the open top end of sample tube 202 when in the upright vertical position and end cap 204a is removed in the carriage 332 for unloading the sample. Plate 350 has a generally flattened and broader body, which may be any suitable shape such as circular, rectilinear (square or rectangular), etc. Movement and operation of the closure plate 350 is controlled by actuator 351 coupled to the plate. Actuator 351 may be any suitable commercially-available electric actuator, pneumatic actuator, or hydraulic actuator. In one embodiment as shown, closure plate 350 may be circular and is pivotably movable via actuator 351 of a rotary type between an inward closed position cover the top end of sample tube 202, and an outward open position uncovering the tube. In other embodiments, closure plate 350 may be slideably movable in a linear manner between the open and closed positions by the actuator 351 of a linear type.

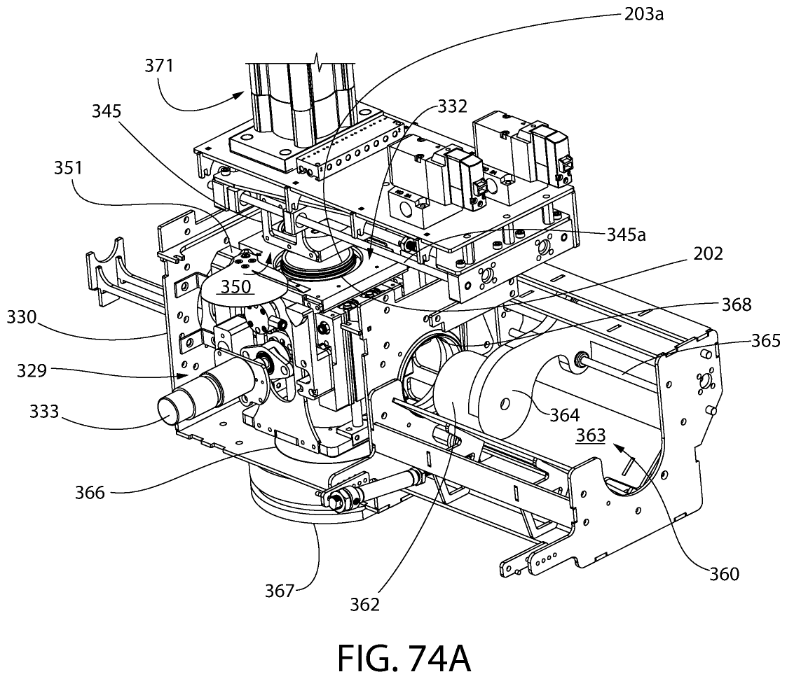

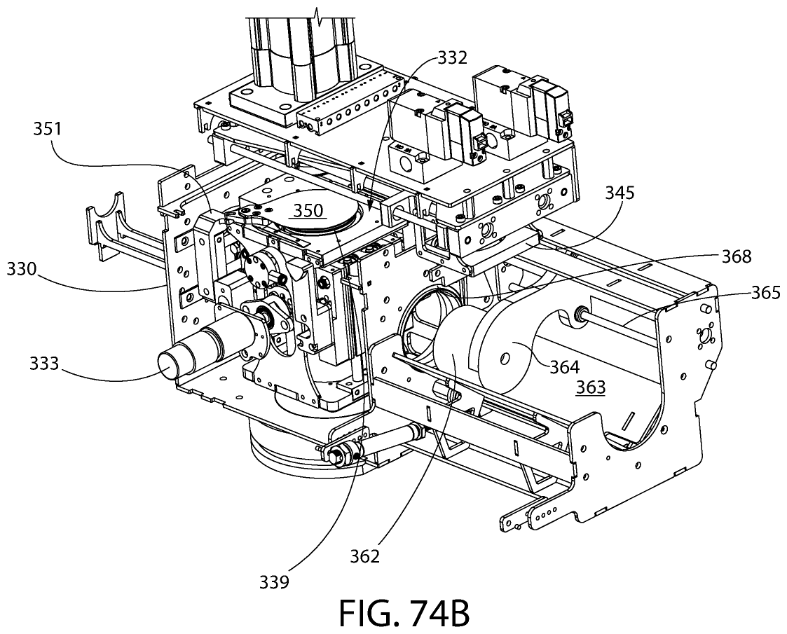

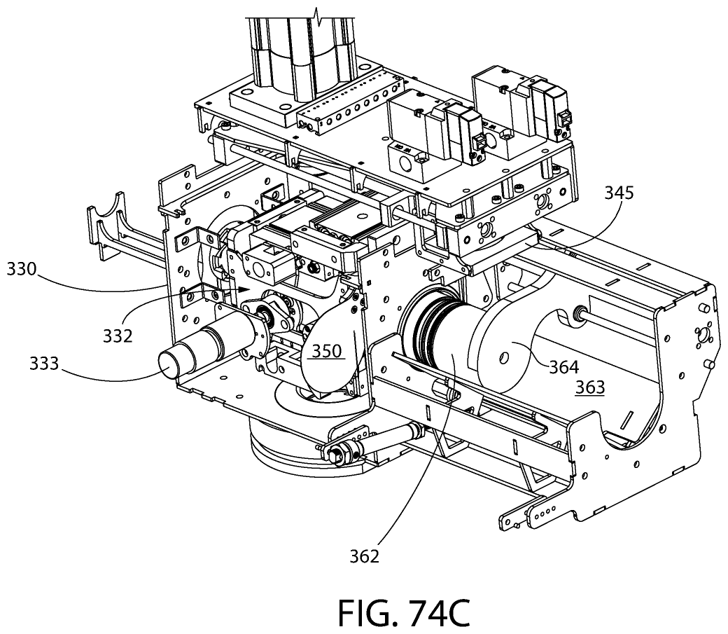

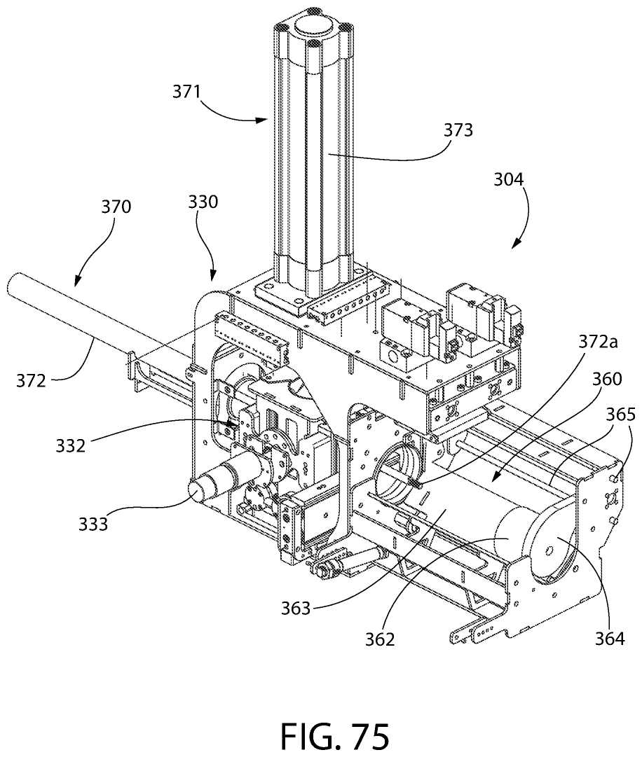

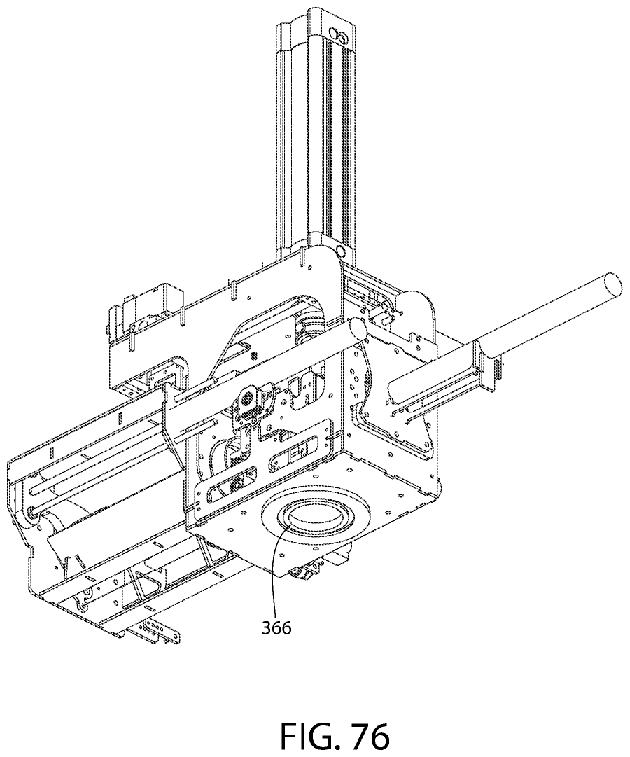

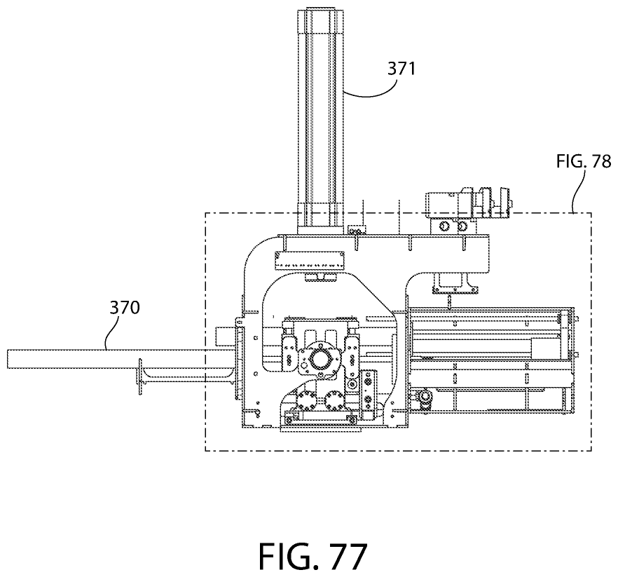

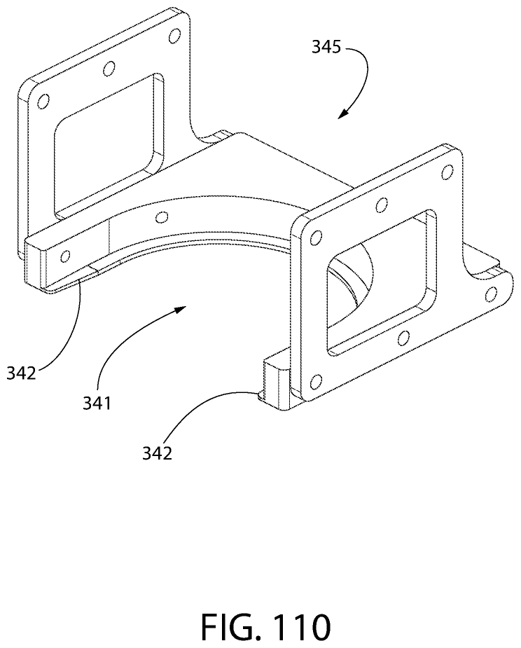

Referring primarily to FIGS. 74A, 96, and 110, carriage 332 further includes a movable decapper 345 which is an apparatus operable to remove detachable end cap 204a from the top end 203a of sample tube 202. In one embodiment, the decapper may be slideably movable in linear manner along feed axis FX on a pair of overhead guide rods 345a; however, pivotable movement may be provided in other embodiments. In addition, decapper 345 may be moveable in linear directions other than along feed axis FX so long as the decapper may engage and remove the end cap 204a. The decapper 345 is suspended from the guide rods above and projects downwards. The decapper may include a flattened plate-like body defining a C-shaped concave recess 341 facing towards carriage 332 and configured to slideably receive and engage the top end cap 204a of sample tube 202. A pair of opposed inwardly projecting rails 342 are formed within in the recess (e.g., one rail on each side) which engage a complementary configured circumferential groove 347 in the sample tube top end cap 204a (best shown in FIG. 110).

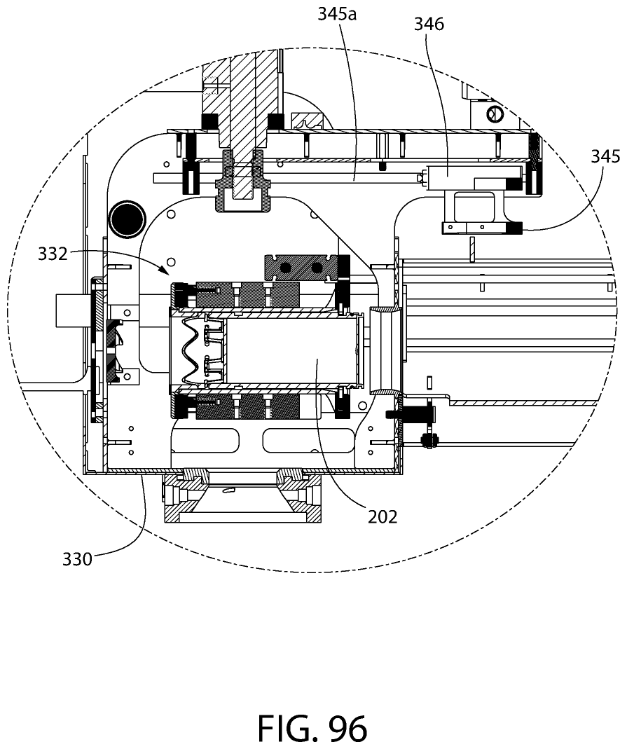

The decapper 345 is movable between an inward position towards receptacle 336 of carriage 332 to engage cap 204a of a newly loaded sample tube 202, and an outward position away therefrom via operation of an actuator 346 (see, e.g., FIG. 96). Actuator 346 may be any suitable commercially-available electric actuator, pneumatic actuator, or hydraulic actuator. In operation, the decapper initially in the outward position moves and engages end cap 204a. Specifically, rails 342 of the decapper 345 engage groove 347 on opposite circular sides of the cap. With the cap thus engaged, the carriage 332 moves from the outward to inward position which uncouples and removes the cap from the sample tube 202. In one embodiment, the end cap 204a may be retained on and sealed to sample tube 202 via a snap fit, which in one embodiment may be formed by circumferentially-extending and inward projecting snap protrusion 343 formed on the sides of lid 204a which resiliently engages corresponding snap groove 344 formed on the tube 202 adjacent to top end 203a (see, e.g., FIG. 55). End cap 204a is formed of a plastic material in one embodiment which has the required elastic deformation properties necessary to form the snap fit to the sample tube body.

To load sample tubes 202 into and from the carriage 332, unloading apparatus 304 includes a tube loading mechanism 360. The mechanism in one embodiment comprises loading actuator 361 including a linearly and horizontally movable ramrod 362 and a tube chute 363. Ramrod 362 may be coupled to actuator 361 via mounting plate 364 slideably movable on a pair of guide rods 365. The actuator 361 (shown schematically in dashed lines FIG. 78) may be coupled to and operable to move mounting plate 364 with ramrod 362 in a linear bi-directional manner towards and away from the carriage 332. Actuator 361 may be any suitable commercially-available linear electric actuator, pneumatic actuator, or hydraulic actuator operable to move plate 364 and ramrod 362 coupled thereto inwards and outwards relative to carriage 332. Tube chute 363 is configured to slideably engage the cylindrical walls 202a of the sample tube. The tube chute receives the loaded sample tube 202 from staging rack 302 via transfer mechanism 306 previously described herein. In one embodiment, chute 363 may be V-shaped in cross section being comprised of two flat plates disposed at an acute angle to each other. In one embodiment, tube chute 363 is pivotably mounted at one end to unloading apparatus 304 and movable for discharging/discarding the empty sample tube after the sample has been unloaded, as further described herein. Tubes are loaded into carriage 332 by ramrod 362 through a loading port 368 formed in housing 330 of the unloading apparatus.

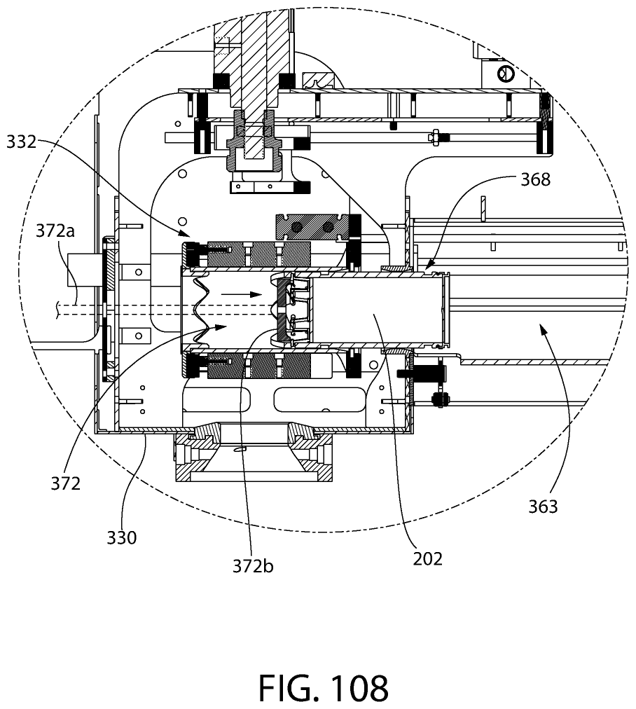

Unloading apparatus 304 further includes a sample tube ejector 370 and sample ejector 371. Tube ejector 370 is configured and operable to push the empty sample tube back out of the carriage tube receptacle 336 after the sample is unloaded. In one embodiment, the tube ejector comprises a horizontally movable piston-plunger 372 which may each be any suitable type commercially available electric linear rod actuator, pneumatic cylinder, or hydraulic cylinder with retractable/extendible operating rod 372a terminated with a diametrically enlarged cylindrical plug disk 372b (see, e.g., FIG. 108). In one embodiment, piston-plunger 372 is coaxially aligned with tube feed axis FX and arranged directly opposite ramrod 362 on the lateral side of carriage 332 opposite the lateral side where the ramrod is located. Ramrod 362 is also coaxially aligned with feed axis FX. Accordingly, in this embodiment, the sample tube 202 is both loaded into and unloaded from receptacle 336 of the tube carriage 332 along the same linear path coaxially with feed axis FX which passes through the geometric center of the receptacle interior space when in its horizontal position in the carriage. In operation, cylindrical plug disk 372b of piston-plunger 372 is configured to fit inside sample tube 202 to abuttingly engage push cap 204b and force the sample tube back out through loading port 368 in housing 330 of the unloading apparatus.

Sample ejector 371 is configured and operable to eject the sample from the sample tube 202 when in its inverted vertical position when the detachable end cap 204a is removed from the top end of the tube. Sample ejector 371 in one embodiment comprises a vertically movable piston-plunger 373 which may each be any suitable type commercially available electric linear rod actuator, pneumatic cylinder, or hydraulic cylinder with retractable/extendible operating rod 373a terminated with a diametrically enlarged cylindrical plug disk 373b. Plug disk 373b is configured for insertion into and through the sample tube 202 from end to end to eject and unload the agricultural sample therefrom.

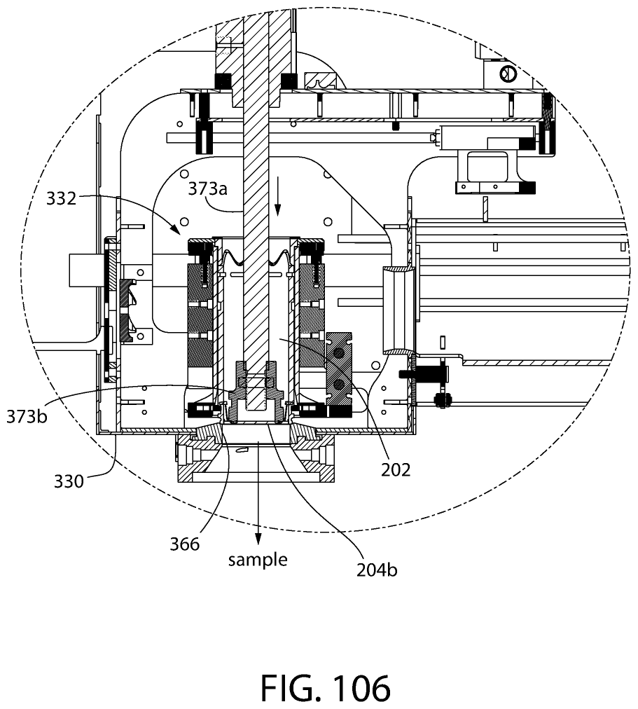

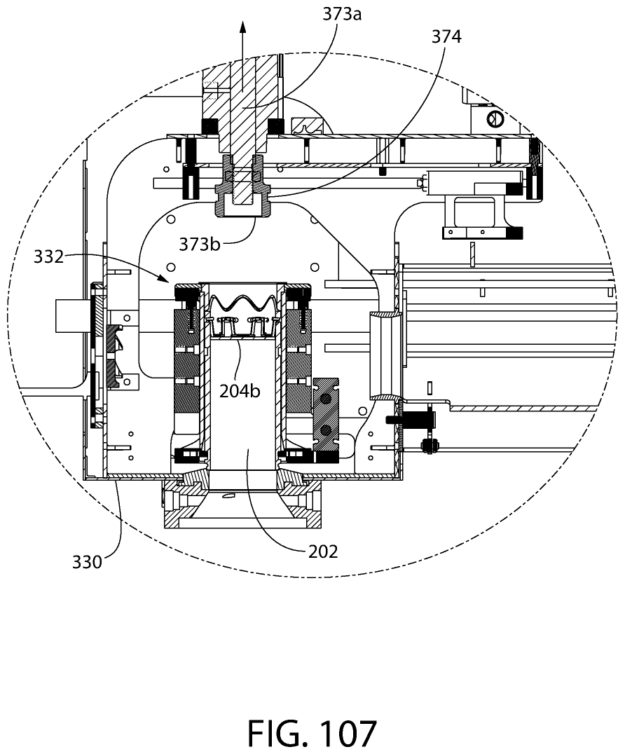

In one embodiment, plug disk 373b is configured to selectively and lockingly engage the slideable push cap 204b of sample tube 202. The plug disk includes a circumferential groove 374 on its exterior surface (see, e.g., FIG. 107) which is engageable with an inward projecting tab 205b on the spring-action retention protrusions 205 of cap 204b (see, e.g., FIG. 50). In one embodiment, tabs 205b are formed on the free terminal ends 205a of the retention protrusions opposite the outward projecting tabs 205c which engage the retention slots 202c in the sample tube wall 202a (see also FIG. 45). In operation, the sample ejector piston-plunger 373 is operable in a linear downward stroke to both push and slide tube cap 204b downwards inside the sample tube 202 when inverted in carriage 332 toward its top end 203a (see, e.g., FIG. 106), and then in a linear n upwards stroke pull and return the cap back upwards to re-lock the cap to the retention slots 202c in the tube (see, e.g., FIG. 107), as further described herein. Engagement between groove 374 of plug disk 373b and tab 205b of push cap 204b enables return of the cap back upwards with the plug disk. It bears noting that the retention protrusions 205 of push cap 204b are pressed and deflected radially inwards when the outward protruding locking tabs 205c are disengaged from retention slots 202c in sample tube 202 as the sample ejector plug disk 373b pushes downwards on the push cap. The inwardly deflected retention protrusions 205 can resiliently spring back outward since the outward protruding locking tabs 205c are maintained inwards by the inside wall surfaces of the sample tube. This in turn maintains between circumferential groove 374 of plug disk 373b and tab 205b of the push cap retention protrusions 205 as the cap slides downwards and upwards inside the sample tube 202 until the retention protrusions reach the tube retention slots 202c and are then resiliently biased back outwards.

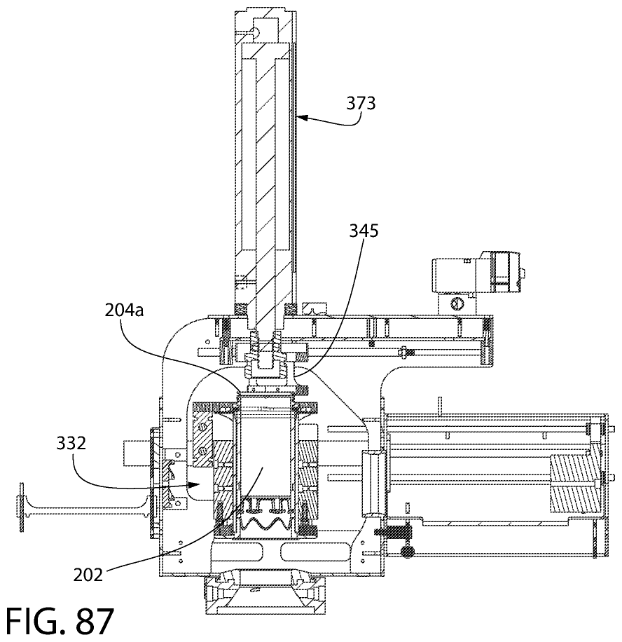

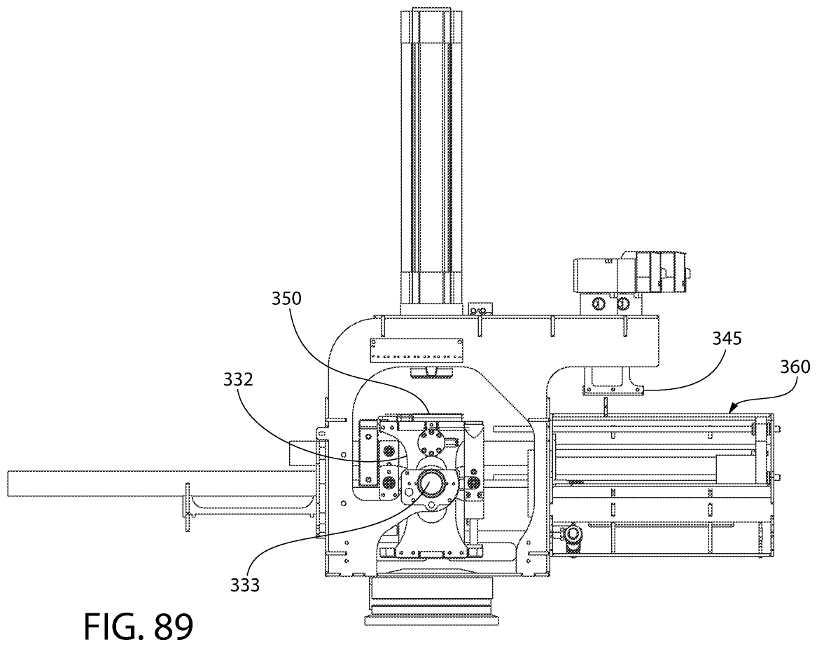

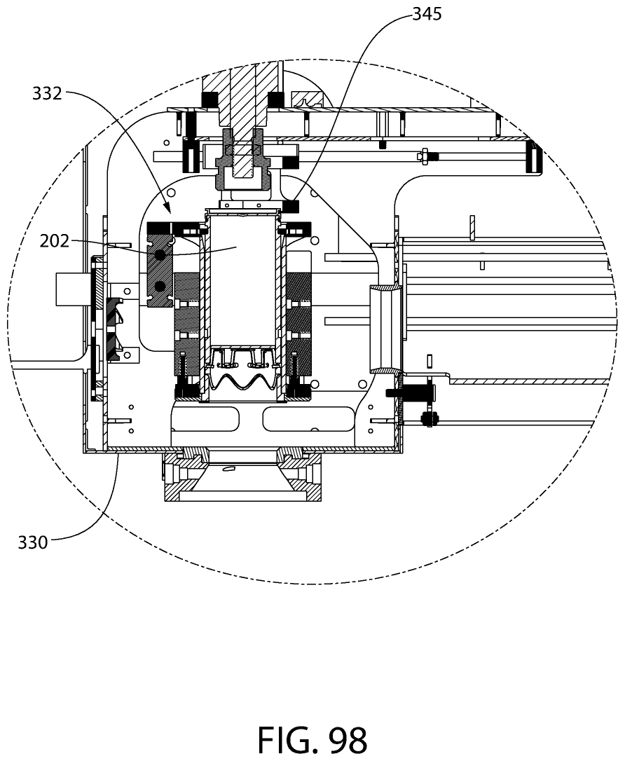

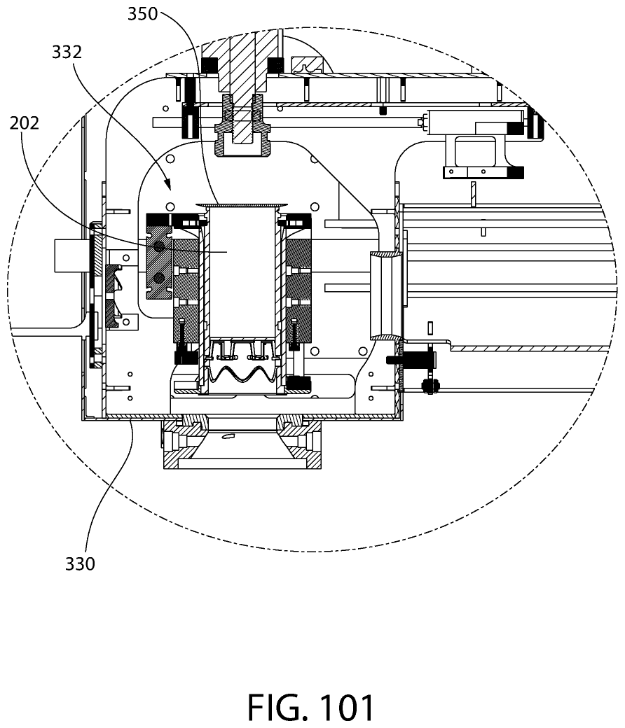

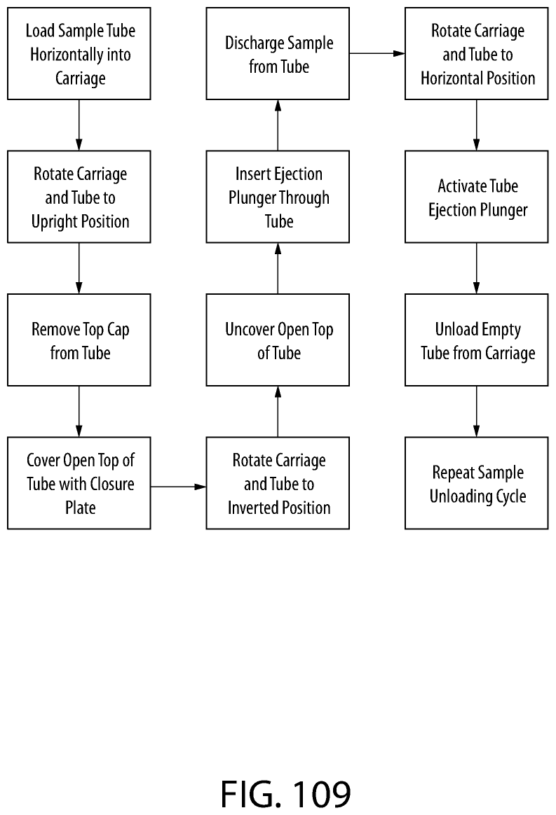

A method or process for unloading an agricultural sample container will now be described using unloading system 300 and various components thereof previously described disclosed herein. FIG. 109 is a high level flow chart showing the general steps in the method or process. Reference will also be made below to FIGS. 84-108, which show more detail of some of the sequential steps in processing and unloading the sample container. FIGS. 95-108 specifically are enlarged cross-sectional views of unloading apparatus 304 specifically showing the sample container (e.g., sample tube 202 in this embodiment) in receptacle 336 of the tube carriage 332 and its various rotational orientations and axial positions during the sample unloading process. References to rotational movement and directions are from the perspective of the equipment as viewed in these foregoing figures, for convenience of description.

Prior to the specific steps of the sample unloading process described below which are performed by unloading apparatus 304, the staging rack 302 and transfer mechanism 306 first perform their functions previously described herein to stage and then load a sample tube 202 onto feed chute 363 of the unloading apparatus 304. These tube staging and feed steps may include first (1) system controller 2820 executes the preprogrammed control logic/program instructions (software) to select the feed ramp 311 from which one of the sample tubes 202 is to be released for loading into sample unloading apparatus 304. (2) The feed gate actuator 314c actuates the rotatable feed gate 314 of the selected “feed ramp” to release the selected sample container to the sample tube elevator 320. (3) Elevator 320 carries the selected sample tube 202 to either the elevator tube lifting member 324, or in some embodiments a vertical position or location along the staging rack 302 where the sample ID on the sample tube can be scanned by the RFID, visual, or other type electronic reader (as described elsewhere herein). If the sample ID is recognized, the authenticated sample tube is loaded into unloading apparatus and the process continues to the first step of sample unloading process in the flow chart in FIG. 109 and as further described below starting with FIG. 95. If sample ID is not recognized for some reason, the sample tube is rejected and ejected from feed chute 363 of the tube feed mechanism 360 after being loaded into the unloading apparatus.

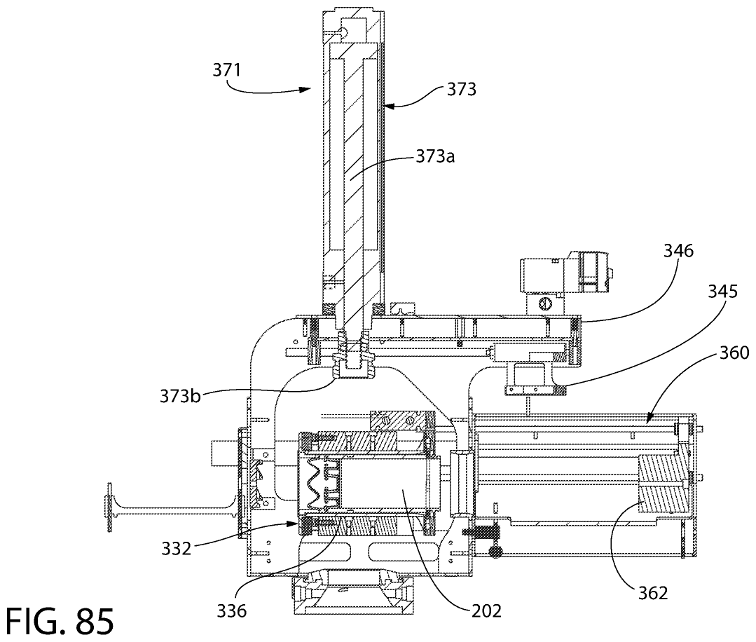

FIG. 95 now shows the accepted capped sample container 200 in the form of the authenticated sample tube 202 which has been loaded onto feed chute 363 of tube loading mechanism 360 from the staging rack 302 by transfer mechanism 306; each previously described herein. The sample tube contains the agricultural sample (e.g., soil or other) and oriented on the slide so that bottom end 203b is facing left towards carriage 332 of the apparatus. Carriage 332 and concomitantly receptacle 336 is rotated 90 degrees clockwise to the right in a horizontal position so that the open top 339 of the receptacle faces towards the feed chute and is readied for receiving the sample tube (see also FIG. 84). The sample tube is being pushed and loaded into receptacle 336 bottom end first by ramrod 362 of the tube loading mechanism 306. FIGS. 85 and 96 show the sample tube fully inserted in the carriage. Castellated support surface 337 in tube receptacle 336 engages complementary configured castellated bottom end 203b of the tube 202.

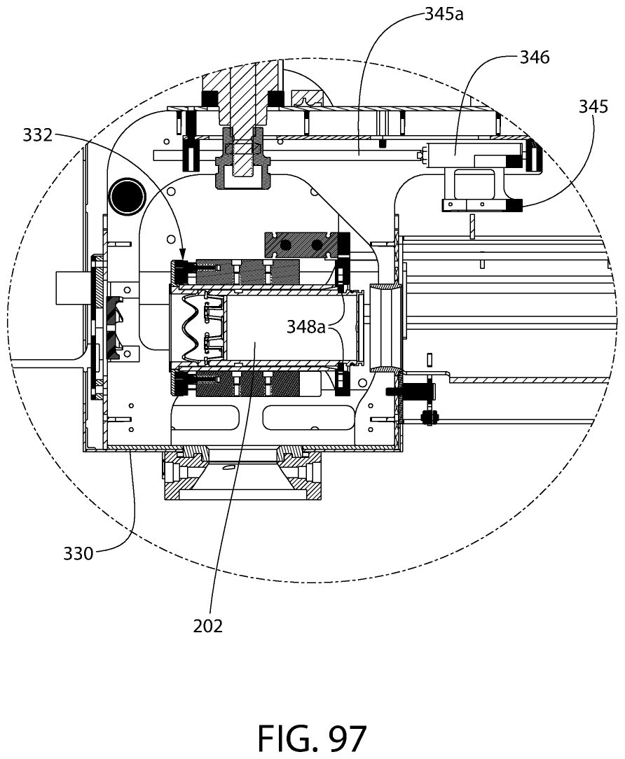

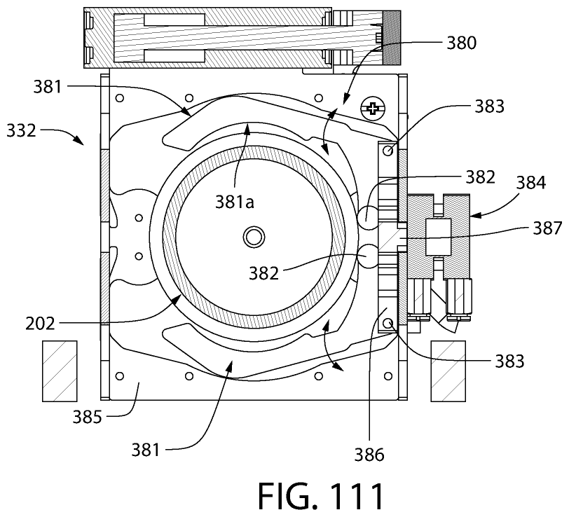

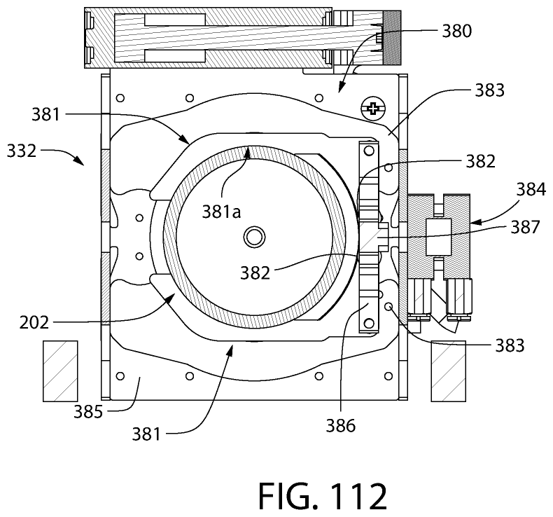

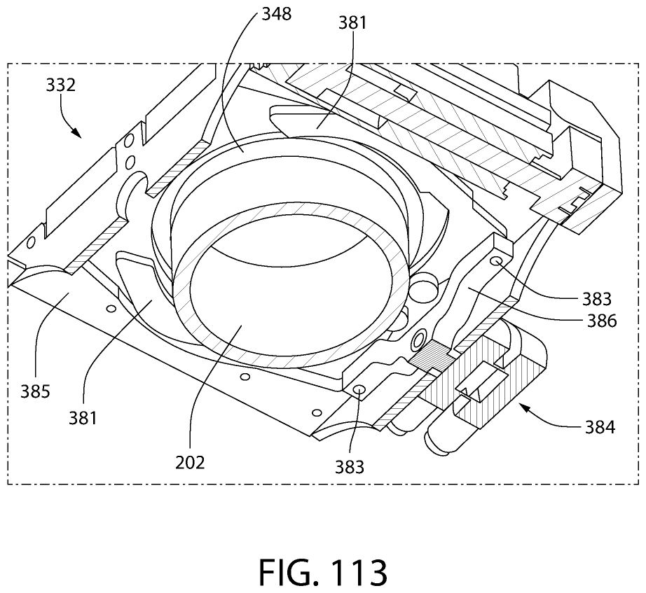

Next, with the sample tube 202 now docked in receptacle carriage 332, FIG. 97 shows movable gripping arms 381 of a sample container or tube gripper 380 of carriage 332 moving inwardly to engage circumferential retention groove 348 formed on the exterior surface of sample tube 202 via a pivotable clamping action of the arms. This retains and locks the tube in receptacle 336 of the carriage when inverted for unloading the sample. With additional reference to FIGS. 111-113 showing the gripper in more detail, each gripping arm 381 of tube gripper 380 is an elongated structure rotatably mounted to the flat top plate 385 of carriage 332 by a pivot bolt 382 (e.g., should bolt). Each gripping arm 381 of the gripping mechanism is further pivotably coupled to a separate pivot pin 383 carried by an elongated bar-shaped actuation member 386. Actuation member 386 is linearly movable inwards and outwards towards or away from sample tube 202 when positioned in tube receptacle 336 of the carriage 332 via operation of a linear actuator 384. Actuator 384 may be any suitable commercially-available electric linear rod actuator, pneumatic actuator, or hydraulic actuator with retractable/extendible operating rod 387 coupled to actuation member 386. Pivot pins 383 are located outboard of the pivot bolts 382 as shown such that moving actuation member 386 inwards and outwards concomitantly produces an openable/closeable claw-like action of the gripping arms 381. The gripper 380 is moveable via actuator 384 between (1) an inwards closed position in which the gripping arms simultaneously move inwards to engage retention groove 348 on the sample tube 202, and (2) an outward open position in which the gripping arms move outwards to disengage the sample tube. Each gripping arm 381 may include an inward facing and arcuately curved grip surface 381a engageable with groove 348 in the sample tube body to lock the tube in receptacle 336 of carriage 332.

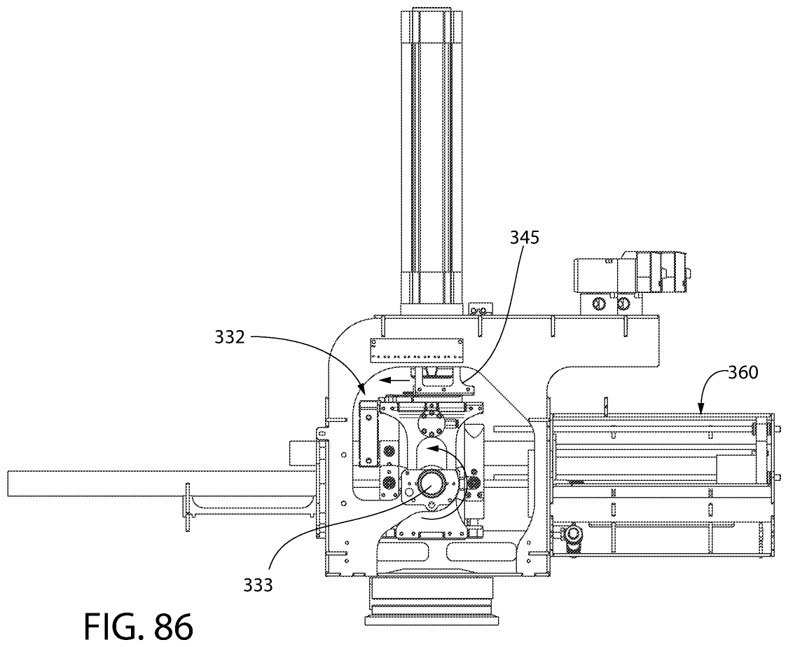

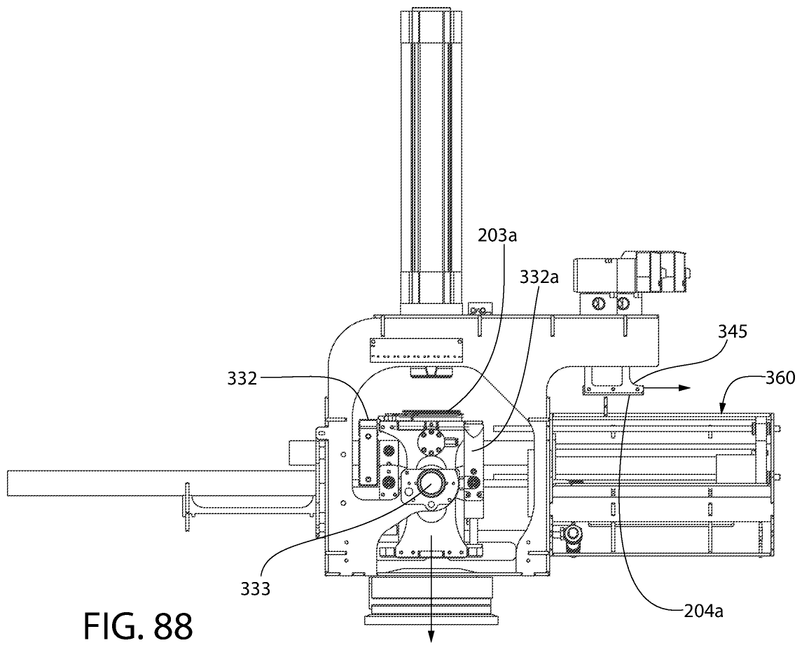

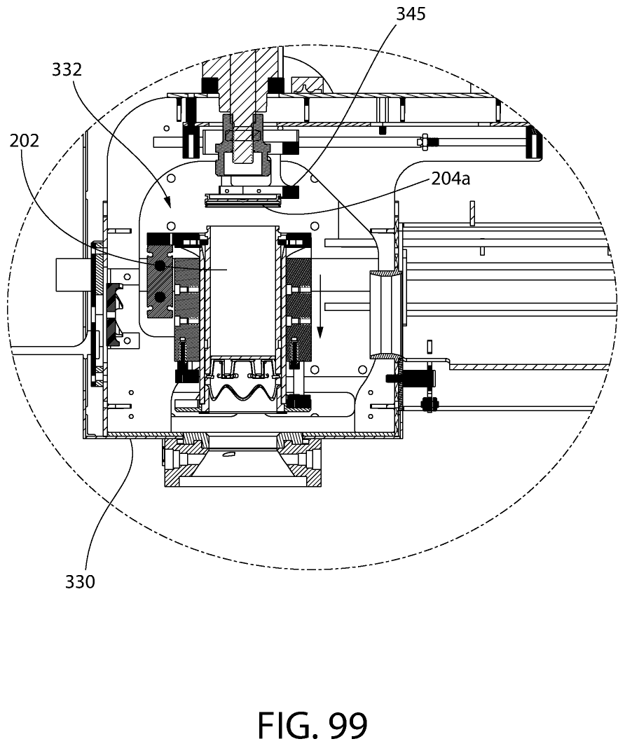

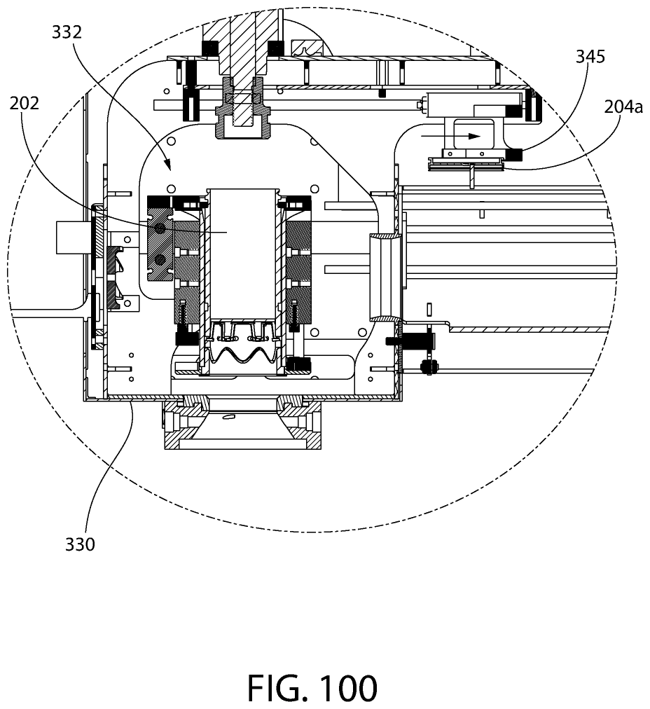

FIGS. 86-87 and 98 next shows the carriage which has been rotated 90 degrees counterclockwise to its upright vertical position. Detachable tube end cap 204a is now positioned at top of the sample tube 202 in the carriage. The decapper 345 has moved inwards to engage the end cap as shown. Rails 342 of the decapper slideably engage groove 347 in cap 204a as previously described herein. With the cap thus engaged, the tube carriage 332 and concomitantly sample tube 202 are moved downwards and inwards relative to carriage housing 330 and its rotating drive shaft 333 (FIGS. 88 and 99) via linear actuators 332a; one each coupled to each side of carriage 332 in one embodiment. Actuators 332a may be any suitable commercially-available electric actuator, pneumatic actuator, or hydraulic actuator configured and operable to slideably move carriage 332 inwards and outwards relative to carriage housing 330 and carriage drive shaft 333. The downward shift in position of the carriage relative to decapper 345 with cap 204a engaged thereto which remain stationary uncouples and “pops” the snap-fit cap 204a off the tube. The decapper 345 with cap 204a then moves back outwards from the carriage 332 with the cap (see, e.g., FIG. 100). The cap may be dropped into an available bin for reuse later during another sample collection run. Alternatively the reverse process could be conducted after the sample has been unloaded to re-install the cap back on the tube. This advantageously allows the operator to handle only the tube assembly without having to handle the caps separately, primarily but not limited to when tubes are being loaded.

To cover the now open top end 203a of sample tube 202 with exposed sample, closure plate 350 moves inwards over the top end 203a to temporarily enclose the tube and its contents (see, e.g., FIGS. 74A-B, 89, and 101 showing plate 350 moving from outward to inward position).

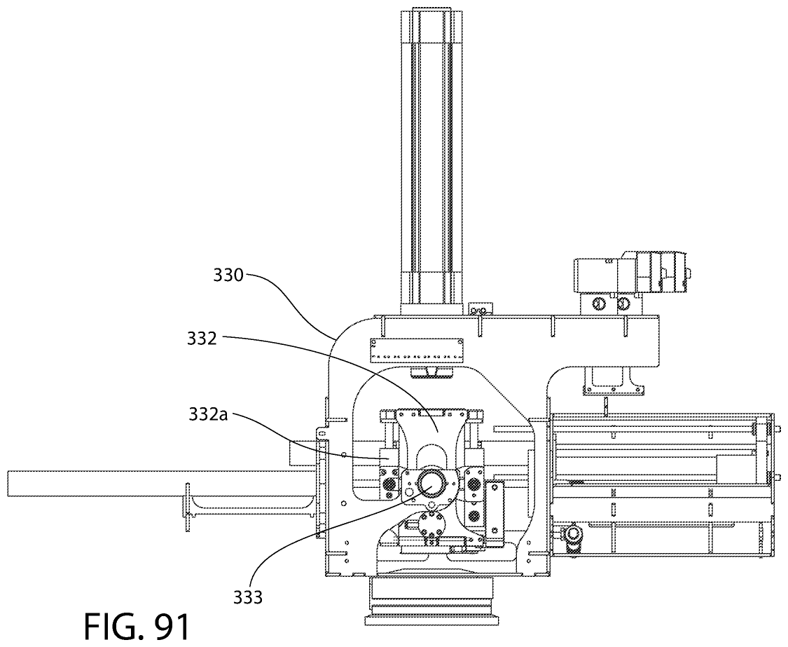

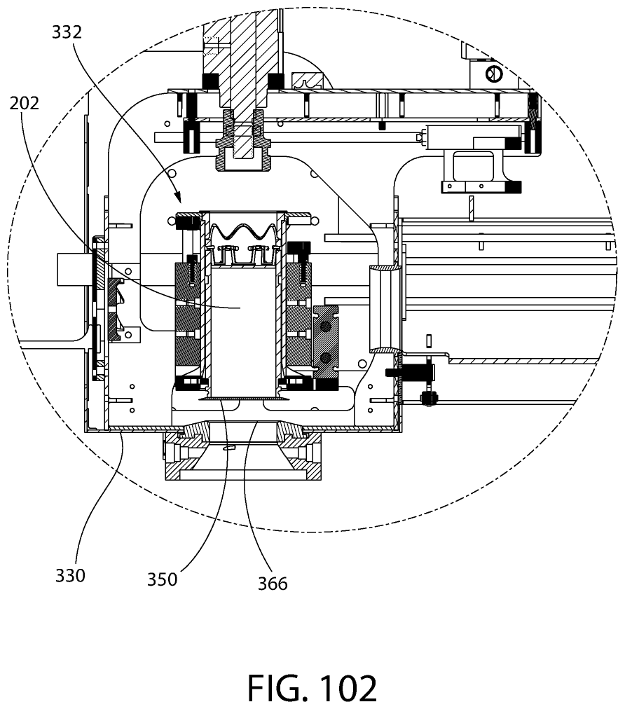

Carriage 332 is then rotated 180 clockwise from its upright vertical position to the inverted vertical position while closure plate 350 covers the now inverted sample tube 202 to prevent or minimize leakage of the sample from the receptacle 336. FIG. 90 shows carriage 332 part way through the rotation in an angled position relative to tube feed axis FX. The full inverted position is shown in FIGS. 91 and 102.

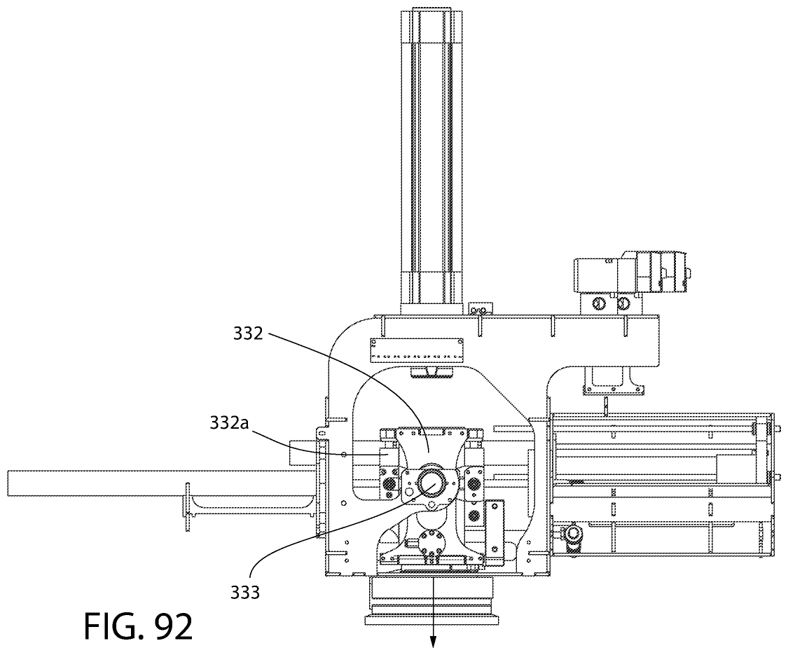

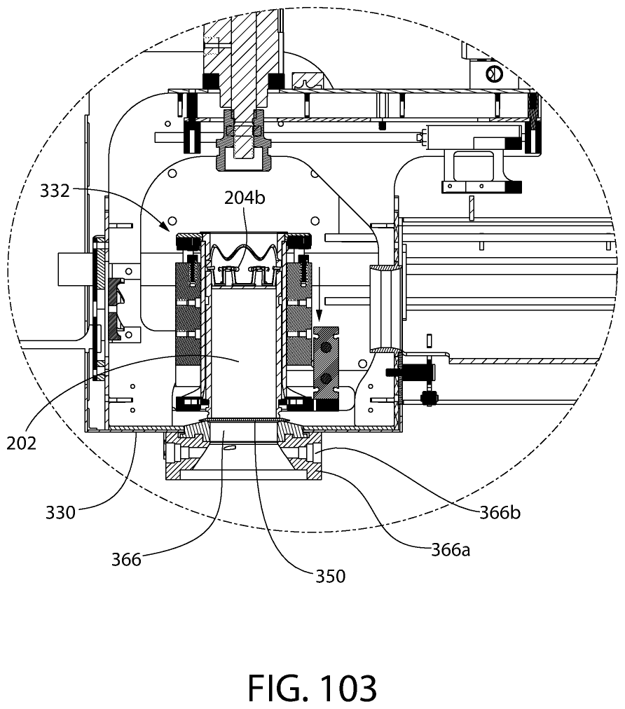

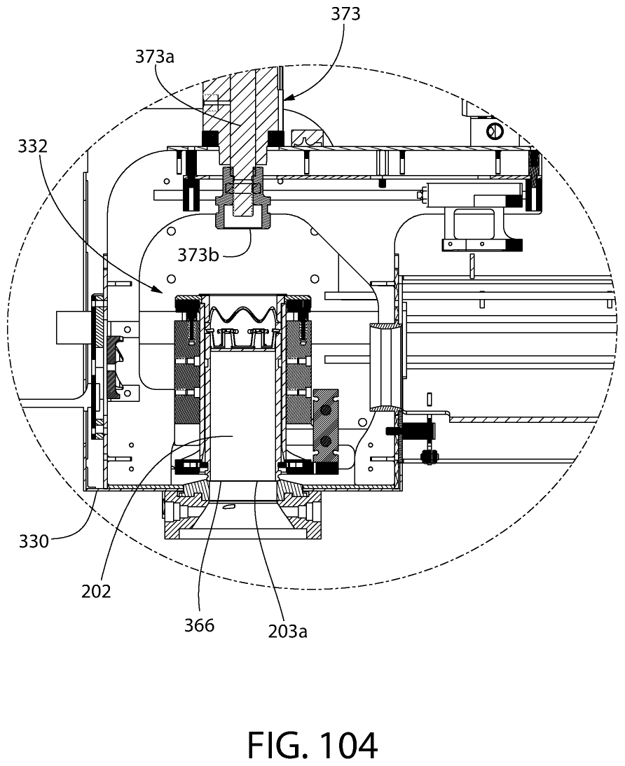

Next, the inverted carriage 332 is moved vertically downwards and outward relative to carriage housing 330 and drive shaft to position the concomitantly inverted top end 203a of sample tube 202 closer to and adjacent sample unloading port 366 at the bottom of the carriage housing 330 (see, e.g., FIGS. 92 and 103). Unloading port 366 is open upwards and downwards as shown. The unloading port 366 may be defined by a block structure 366a attached to the bottom of carriage housing 330 and may include one or more nozzles 366b configured for coupling to a water source to clean out soil from the port and associated passageway through the block structure between each sample unloading cycle. The closure plate 350 is next moved outwards to uncover the open end of the sample tube 202 which is now exposed to the unloading port 366 (see, e.g., FIG. 104).

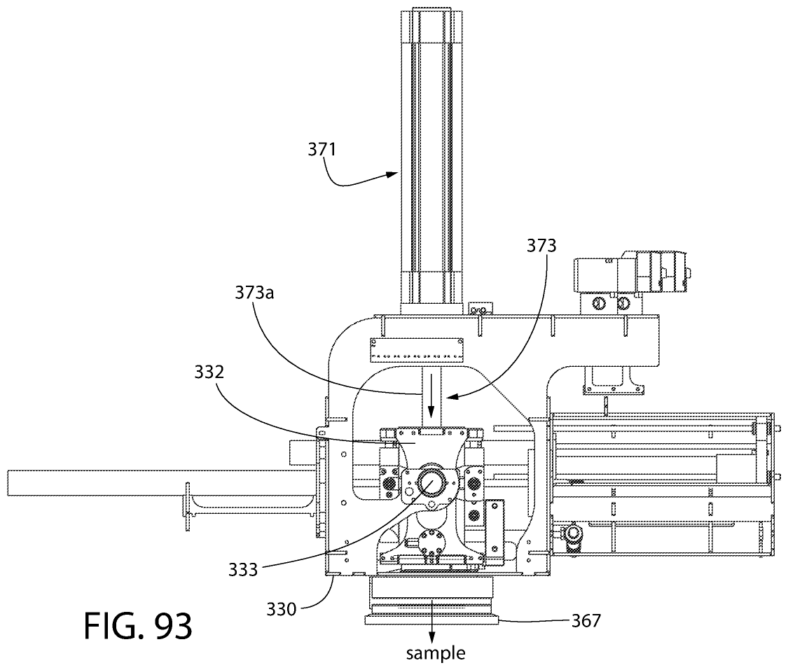

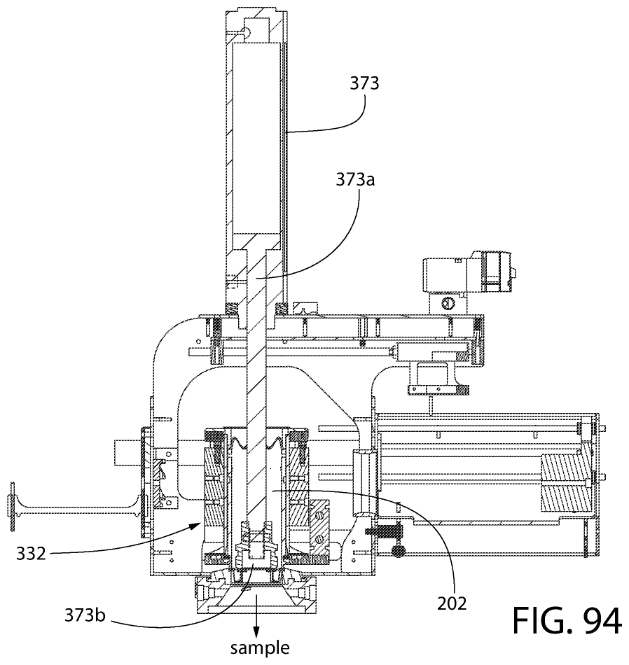

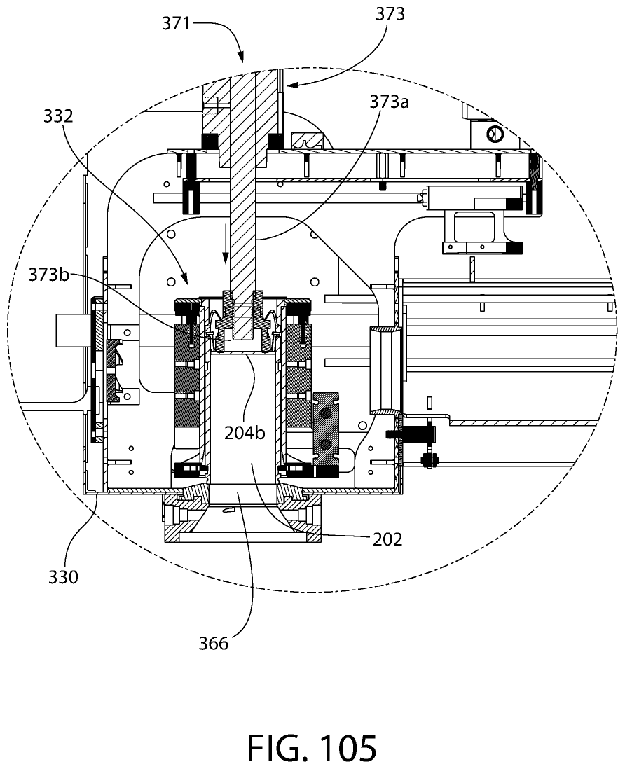

Next, the sample ejector 370 is activated to eject the sample from the sample tube (see, e.g., FIGS. 93 and 105). The sample ejector piston-plunger 373 moves vertically downwards and enters the bottom end 203b of the tube to lockingly engage slideably push cap 204b. Specifically, circumferential groove 374 in plug disk 373b of the piston-plunger lockingly engages the inward projecting tabs 205b on the spring-action retention protrusions 205 of cap 204b (FIG. 105). Engagement between the retention protrusions 205 and inside surfaces of the sample tube 202 forces the protrusions inwards which maintains locking engagement between groove 374 in the plug disk and tabs 205b. Piston-plunger 373 unseats the cap 204b from retention slots 202c in the sample tube walls 202a as it continues to move downward fully in the tube with the cap 204b to eject the entire sample from the tube (see, e.g., FIGS. 94 and 106). The piston-plunger may be cycled upwards and downwards within sample tube 202 two or more times to ensure the entire contents of the tube are emptied. After the last downward stroke, the piston-plunger draws the cap 204b back upwards and reseats/relocks the cap in the retention slots 202c of sample tube 202. The piston-plunger is completely withdrawn from the tube carriage 332 to allow the carriage to be rotated (see, e.g., FIG. 107).

The sample material ejected by the ejector piston-plunger 373 falls via gravity into the unloading funnel 367 positioned beneath unloading port 366. The funnel directs the sample to the sample preparation system (shown schematically by dashed lines FIG. 78), which may include a sample slurry preparation chamber 369 for preparing a slurry by combining the sample material with water via one or more water inlets 369a for further processing and eventual chemical analysis. Chamber 369 includes a slurry outlet 369b. If slurry preparation occurs below the funnel immediately after ejecting the sample, the closure plate 350 may be moved back inwards to re-cover the open-ended sample tube 202 and prevent water from splashing and entering the carriage. If the slurry preparation equipment has its own closure device or the slurry is prepared at a later time or remote from the funnel 367, the closure plate 350 may remain in the outward position.

In either of the above slurry preparation scenarios, the carriage 332 is then rotated 90 degrees counter-clockwise to horizontally align receptacle 336 and sample tube 202 with the tube feed axis FX (see, e.g., FIG. 108). If closure plate 350 is still inward and covering the open top end of the tube and receptacle, the closure plate is moved outwards to open them. Sample tube ejector 370 is then actuated to eject the empty tube. Piston-plunger 372 moves to the right along feed axis FX into carriage 332 through support ring 337 in receptacle 332 and pushes the empty tube outwards to the right and back onto tube feed chute 363 of the loading mechanism which is in a horizontal position. Feed chute 363 is then pivotably rotated to an angular position to dump the empty tube into an available bin or container for re-use. The feed chute is then rotated back to the normal horizontal tube feed position and ready for receiving and unloading the next filled sample tube by repeating the foregoing method/process.

Performance of the foregoing sample tube unloading method/process and operation associated equipment described of the sample unloading system 300 may be automatically controlled by the programmable system controller 2820 shown in FIG. 2 and already described in detail above. System 300 is operably coupled to controller 2820 via suitable wired and/or wireless communication links as shown. For example, once filled sample tubes 202 are manually loaded onto staging rack 302, the simple activation of the control system via a hard or “soft” (software) start button automatically feeds the tubes via transfer mechanism 306 previously described herein into the unloading apparatus 304 from the staging rack. Tubes are loaded into the unloading apparatus in a serial manner one at a time to unload the sample material according to a sequenced tube feed plan preprogrammed into the controller. The tube feed plan establishes the order in which the sample tubes from various feed ramps 311 are staged to be removed from staging rack 302 and fed into the unloading apparatus 304 to unload the samples. The controller thus directs the transfer mechanism 306 to conduct the tube feed and loading into the unloading apparatus according to the preprogrammed feed plan. In various tube feed scenarios of the plan, certain feed ramps 311 may be emptied completely for unloading the sample tubes before proceeding to the next feed ramp, or certain feed ramps may be partially emptied before moving to certain other feed ramps. This allows the user to preprogram the controller 2820 to prioritize processing and unloading of certain sample tubes first. It will therefore be appreciated that numerous different sample tube feeding scenarios and order of processing the tubes may be implemented for various reasons.

System controller 2820 then controls operation of the unloading apparatus 304 to process and unload the contents of each sample tube 202 in the manner described above and shown in FIGS. 84-109 via executing preprogrammed control logic/program instructions (e.g., software). It is well within the ambit of those skilled in the art to code the program instructions and steps disclosed herein to operate the unloading apparatus 304 in the manner described to unload the agricultural sample from the sample tube 202. All unloading apparatus associated equipment described herein may therefore be automatically controlled by the controller so no manual intervention is required other than the user or operator activating the hard or soft start button. In other possible embodiments, a combination of manual and automatically controlled operation and steps may be used.

### Examples

The following are non-limiting examples.

Example A1—A sample unloading system comprising: a sample staging rack 302 comprising at least one inclined feed ramp 311 configured for receiving an elongated sample tube 202 configured for holding the sample, the sample tube including a first end cap 204a and a second end cap 204b; an unloading apparatus 304 coupled to the staging rack, the unloading apparatus configured to receive the sample tube from the staging rack; and a transfer mechanism 306 operable to transfer the sample tube from the staging rack to the unloading apparatus.

Example A2—the system according to any one of Example A1, wherein the unloading apparatus 304 comprises a rotatable carriage 332 configured to hold and rotate the sample tube 202 in opposing directions.

Example A3—the system according to Example A2, wherein the unloading apparatus 304 comprises a loading mechanism 360 operable to load the sample tube 202 into the carriage 332.

Example A4—the system according to any one of Examples A2 or A3, wherein the carriage 332 is operable to rotate the sample tube 202 between an upright vertical position and an inverted vertical position.

Example A5—the system according to Example A4, wherein the carriage 332 is operable to rotate the sample tube 202: 90 degrees in a first direction to the upright vertical position; and 180 degrees therefrom in an opposite second direction to the inverted vertical position.

Example A6—the system according to Example A4, wherein the first end cap 204a is detachably coupled to a first end of the sample tube 202, and the second end cap 204b is slideably movable inside the sample tube towards and away from the first end cap.

Example A7—the system according to Example A6, wherein the unloading apparatus 304 further comprises a decapper 345 operable to remove the first end cap 204a from the sample tube 202 when in its upright vertical position.

Example A8—the system according to Example A7, wherein the unloading apparatus 304 comprises a sample ejector 371 configured to eject the sample from the sample tube 202 when in its inverted vertical position when the first end cap is removed.

Example A9—the system according to Example A8, wherein the sample ejector 371 comprises a vertically movable plunger 373 which engages and slides the second end cap 204b inside the sample tube 202 to eject the sample.

Example A10—the system according to any one of Examples A4-A9, wherein the unloading apparatus 304 comprises a movable closure plate 350 movable between an inward position operable to retain the sample in the sample tube 202 when in its inverted vertical position when the first end cap 204a is removed, and an outward position operable to release the sample from the sample tube when in its inverted vertical position.

Example A11—the system according to Example A10, wherein the closure plate 350 engages an open end of the sample tube 202 created when the first end cap is removed.

Example A12—the system according to Example A11, wherein the closure plate 350 is further operable to engage the open end of the sample tube 202 when in its upright vertical position.

Example A13—the system according to any one of Examples A1-A12, wherein the staging rack 302 comprises a plurality of inclined feed ramps 311 arranged in parallel relationship to each other.

Example A14—the system according to Example A13, wherein the transfer mechanism 306 comprises a rotary feed gate 314 configured to grasp the sample tube 202 on the staging rack and pass the sample tube towards the unloading apparatus.

Example A15—the system according to Example A14, wherein the rotary feed gate 314 is positioned at a proximal end of the inclined feed ramp 311 adjacent to the unloading apparatus.

Example A16—the system according to Examples A15, wherein the transfer mechanism 306 comprises a tube elevator 320 comprising a movable chain 321 with lifting member 324 configured to engage and load the sample tube 202 into the unloading apparatus 304 from the staging rack 302.

Example A17—the system according to Example A3, wherein the loading mechanism 360 comprises a ramrod 362 operable to push the sample tube 202 into a receptacle 336 of the carriage 332.

Example A18—the system according to Example A17, wherein the unloading mechanism 360 comprises an unloading plunger 372 operable to push the sample tube 202 out of the receptacle 336 of the carriage 332.

Example A19—the system according to Examples A17 or A18, wherein the unloading mechanism 304 comprises a feed chute 363 pivotably coupled to the unloading apparatus.

Example A20—the system according to any one of Examples A1-A19, further comprising a programmable controller 2820 operably coupled to and configured to control operation of the unloading apparatus 304 and transfer mechanism 306.

Example A21—the system according to any one of Examples A1-A20, wherein the sample is a soil sample.

Example A22—A method for unloading a sample container 200 comprising: inserting a capped sample tube 202 containing a sample into a rotatable carriage 332 of an unloading apparatus 304; rotating the sample tube 202 a first time to an upright vertical position; uncapping the sample tube which creates an open top end 203a; rotating the sample tube a second time to an inverted vertical position; and ejecting the sample from the sample tube.

Example A23—the method according to Example A22, wherein the carriage 332 includes an elongated receptacle 336 into which the sample tube 202 is inserted.

Example A24—the method according to Examples A22 or A23, further comprising after the uncapping step, a step of covering the uncapped sample tube 202 with a closure plate 350 before rotating the sample tube the second time.

Example A25—the method according to Example A24, further comprising before the ejecting step, a step of uncovering the uncapped sample tube 202 by removing the closure plate 350.

Example A26—the method according to Example A22, wherein the ejecting step comprises inserting a sample ejector piston-plunger 373 through the sample tube 202 in a downward stroke.

Example A27—the method according to Example A26, wherein the sample ejector piston-plunger 373 lockingly engages a push cap 204b slideably disposed inside the sample tube 202 and moves the push cap downwards towards the open top end 203a of the sample tube to eject the sample.

Example A28—the method according to Example A27, further comprising after the ejecting step, a step of moving the sample ejector piston-plunger 273 in an upward stroke which draws the push cap 204b back upwards in the sample tube 202.

Example A29—the method according to Example A23, wherein the uncapping step includes engaging a decapper 345 with a snap-fit cap 204a on the top end 203a of the sample tube 202, and moving the carriage 332 to lower the tube with the decapper engaged with the snap-fit cap to remove the cap.

Example A30—the method according to Example A23, wherein inserting step comprises engaging a castellated bottom end 203b of the sample tube 202 with a mating castellated surface 337 inside the receptacle 336.

Example A31—the method according to Examples A23 or A30, wherein the inserting step includes pushing the sample tube 202 into the receptacle 336 with a ramrod 362 slideably movable on the unloading apparatus 304.

Example A32—the method according to Example A31, wherein the ramrod 362 engages the sample tube 202 on a V-shaped feed chute 363 aligned with a loading port 368 of the unloading apparatus 304 to guide the sample tube into the receptacle 336.

Example A33—the method according to Example A23, further comprising after the ejecting steps of rotating the sample tube 202 a third time to a horizontal position, and pushing the sample tube out of the unloading apparatus 304 with a tube ejector piston-plunger 372.

Example A34—the method according to Example A33, wherein the sample tube 202 is both inserted into the unloading apparatus 304 and pushed out of the unloading apparatus in opposing linear directions along a common feed axis FX.

Example A35—the method according to any one of Examples A22-A34, further comprising a programmable controller 2820 operably coupled to the unloading apparatus 304 and configured to control operation of the unloading apparatus.

Example A36—the method according to Example A22, wherein the sample comprises agricultural sample material.

Example A37—the method according to Example A36, wherein the sample material comprises soil.

Example A38—the method according to Example A22, wherein the carriage 332 is both rotatable vertically to change the sample tube 202 between upright and inverted vertical positions, and linearly movable in a vertical direction to raise or lower the sample tube 202.

Example A39—the method according to Example A38, wherein the carriage 332 is rotatably supported by a horizontally oriented rotating drive shaft 333.

Example A40—the method according to Example A39, wherein the carriage 332 has a rotational range of motion of 360 degrees about the drive shaft 333.

Example A41—A method for unloading a sample container 200 comprising: inserting a capped sample tube 202 containing a sample into an unloading apparatus 304; rotating the sample tube 202 a first time to an upright vertical position; uncapping the sample tube which creates an open top end 203a; rotating the sample tube a second time to an inverted vertical position; and ejecting the sample from the sample tube, wherein the sample comprises a solid material or an agricultural sample material.

Example A42—the method of Example A41, wherein the sample is the solid material.

Example A43-method the of Example A41, wherein the sample is the agricultural sample material.

Example A44—the method of Example A41, wherein the sample is soil.

Example A45—A sample container comprising: an elongated tubular body defining a longitudinal axis LA2, a top end 203a, a bottom end 203b, and an internal cavity 207 extending between the ends configured for holding the sample; a first cap 204a detachably coupled to the top end; and a second cap 204b slideably disposed in the cavity, the second cap being movable in opposing directions between the first and second ends.

Example A46—the sample container according to Example A46, wherein the second cap 204b comprises a base 208 and a plurality of longitudinally-extending retention protrusions 205 extending downwards from the base.

Example A47—the sample container according to Examples A45 or A46, wherein the body further comprises a plurality of circumferentially spaced apart retention slots 202c configured to lockingly engage the retention protrusions 205.

Example A48—the sample container according to Example A47, wherein the retention protrusions 205 are outwardly flared and form outward protruding locking tabs 205c which engage the retention slots 202c.

Example A49—the sample container according to Examples A47 or A48, wherein retention slots 202c are located proximate to the bottom end 203b of the tubular body.

Example A50—the sample container according to Example A48, wherein the retention protrusions 205 each further comprise an inward projecting tab 205b.

Example A51—the sample container according to Example A45, wherein the tubular body defines a pair of longitudinally spaced apart circumferential grooves 348, 349 formed in an exterior surface of the tubular body.

Example A52—the sample container according to Example A45, wherein the first cap 204a comprises an external circumference groove 347.

Example A53—the sample container according to Example A52, wherein the first cap 204a is snap-fit to top end of the tubular body via an inwardly projecting annular snap protrusion 343 engageable with a circumferentially-extending snap groove 344 formed in the tubular body.

Example A54—the sample container according to Example A45, wherein the bottom end 206b has an undulated castellated configuration.

The systems, apparatuses, and methods disclosed herein are usable with and may form part of an overall agricultural sampling and analysis systems, such as but not limited to those described in U.S. Patent Application Publication No. 2018/0124992A1 and PCT Publication No. WO2020/012369, and other systems are described in U.S. Application Nos. 62/983,237, filed on 28 Feb. 2020; 63/017,789, filed on 30 Apr. 2020; 63/017,840, filed on 30 Apr. 2020; 63/018,120, filed on 30 Apr. 2020; 63/018,153, filed on 30 Apr. 2020; 63/191,147, filed on 20 May 2021; 63/191,159, filed on 20 May 2021; 63/191,166, filed on 20 May 2021; 63/191,172, filed on 20 May 2021; Ser. No. 17/326,050, filed on 20 May 2021; 63/191,186, filed on 20 May 2021; 63/191,189, filed on 20 May 2021; 63/191,195, filed on 20 May 2021; 63/191,199, filed on 20 May 2021; 63/191,204, filed on 20 May 2021; Ser. No. 17/343,434, filed on 9 Jun. 2021; 63/208,865, filed on 9 Jun. 2021; Ser. No. 17/343,536, filed on 9 Jun. 2021; 63/213,319, filed on 22 Jun. 2021; 63/260,772, filed on 31 Aug. 2021; 63/260,776, filed on 31 Aug. 2021; 63/260,777, filed on 31 Aug. 2021; 63/245,278, filed on 17 Sep. 2021; 63/264,059, filed on 15 Nov. 2021; 63/264,062, filed on 15 Nov. 2021; 63/264,065, filed on 15 Nov. 2021; 63/268,418, filed on 23 Feb. 2022; 63/268,419, filed on 23 Feb. 2022; 63/268,990, filed on 8 Mar. 2022; 63/269,060, filed 9 Mar. 2022; 63/269,064, filed 9 Mar. 2022; 63/365,243, filed 24 May 2022; 63/365,244, filed 24 May 2022; 63/366,673, filed 20 Jun. 2022; 63/366,674, filed 20 Jun. 2022; 63/369,722, filed 28 July 2022; 63/369,724, 28 Jul. 2022; 63/369,765, filed 28 Jul. 2022; 63/369,988, filed 1 Aug. 2022; 63/370,072, filed 1 Aug. 2022; 63/370,077, filed 1 Aug. 2022; 63/370,081, filed 1 Aug. 2022; and PCT/IB2021/051076, filed on 10 Feb. 2021; PCT Application Nos. PCT/IB2021/051077, filed on 10 Feb. 2021; PCT/IB2021/052872, filed on 7 Apr. 2021; PCT/IB2021/052874, filed on 7 Apr. 2021; PCT/IB2021/052875, filed on 7 Apr. 2021; PCT/IB2021/052876, filed on 7 Apr. 2021. Other sampling systems are described in U.S. Application Nos. 62/983,237, filed on 28 Feb. 2020; 63/017,789, filed on 30 Apr. 2020; 63/017,840, filed on 30 Apr. 2020; 63/018,120, filed on 30 Apr. 2020; 63/018,153, filed on 30 Apr. 2020; PCT/IB2021/051076, filed on 10 Feb. 2021; and PCT Application Nos. PCT/IB2021/051077, filed on 10 Feb. 2021; PCT/IB2021/052872, filed on 7 Apr. 2021; PCT/IB2021/052874, filed on 7 Apr. 2021; PCT/IB2021/052875, filed on 7 Apr. 2021; PCT/IB2021/052876, filed on 7 Apr. 2021.

While the foregoing description and drawings represent some example systems, it will be understood that various additions, modifications and substitutions may be made therein without departing from the spirit and scope and range of equivalents of the accompanying claims. In particular, it will be clear to those skilled in the art that embodiments of the present disclosure may be embodied in other forms, structures, arrangements, proportions, sizes, and with other elements, materials, and components, without departing from the spirit or essential characteristics thereof. In addition, numerous variations in the methods/processes described herein may be made. One skilled in the art will further appreciate that the embodiments of the present disclosure may be used with many modifications of structure, arrangement, proportions, sizes, materials, and components and otherwise, used in the practice of the embodiments of the present disclosure, which are particularly adapted to specific environments and operative requirements without departing from the principles of the present embodiments of the present disclosure. The presently disclosed embodiments are therefore to be considered in all respects as illustrative and not restrictive, the scope of the embodiments of the present disclosure being defined by the appended claims and equivalents thereof, and not limited to the foregoing description or embodiments. Rather, the appended claims should be construed broadly, to include other variants and embodiments, which may be made by those skilled in the art without departing from the scope and range of equivalents of the embodiments of the present disclosure.

1. A sample container comprising:
an elongated tubular body defining a longitudinal axis, a top end, a bottom end, and an internal cavity extending between the ends configured for holding the sample;
a first cap detachably coupled to the top end; and
a second cap slideably disposed in the cavity, the second cap being movable in opposing directions between the top and bottom ends.

2. The sample container according to claim 1, wherein the second cap comprises a base and a plurality of longitudinally-extending retention protrusions extending downwards from the base in a circumferentially spaced apart arrangement.

3. The sample container according to claim 1, wherein the tubular body further comprises a plurality of circumferentially spaced apart retention slots configured to lockingly engage the retention protrusions.

4. The sample container according to claim 3, wherein the retention protrusions are outwardly flared and terminated by outward protruding locking tabs which engage the retention slots.

5. The sample container according to claim 4, wherein the retention protrusions each comprise resiliently deformable legs which are radially moveable inwards and outwards relative to a longitudinal axis of the sample container.

6. The sample container according to claim 4, wherein retention slots are located proximate to the bottom end of the tubular body.

7. The sample container according to claim 5, wherein the retention protrusions each further comprise an inward projecting tab disposed opposite the outward protruding locking tabs.

8. The sample container according to claim 7, wherein the inward projecting tabs are configured for selective engagement by a plunger device insertable into the internal cavity of the tubular body.

9. The sample container according to claim 3, wherein the retention slots each comprises an arcuately curved circumferentially-extending opening formed in a cylindrical wall of the tubular body.

10. The sample container according to claim 1, wherein the tubular body defines at least one circumferential groove formed in an exterior surface of the tubular body.

11. The sample container according to claim 10, wherein the tubular body defines a pair of longitudinally spaced apart circumferential grooves formed in an exterior surface of the tubular body.

12. The sample container according to claim 1, wherein the first cap comprises an external circumference groove.

13. The sample container according to claim 1, wherein the first cap is snap-fit to the top end of the tubular body via an inwardly projecting annular snap protrusion engageable with a circumferentially-extending snap groove formed in the tubular body.

14. The sample container according to claim 1, wherein the bottom end of the tubular body comprises an anti-rotation feature.

15. The sample container according to claim 14, wherein the anti-rotation feature has an undulating castellated configuration.

16. The sample container according to claim 1, wherein the tubular body is cylindrical with a circular cross-sectional shape.

17. The sample container according to claim 1, wherein the second cap is spaced inwards within the internal cavity from the bottom end of the tubular body.

18. The sample container according to claim 4, wherein the second cap is a pushable cap configured for engagement by a plunger device insertable into the internal cavity of the tubular body and operable to move and disengage the locking tabs of the retention protrusions from the retention slots of the tubular body.

19. The sample container according to claim 4, wherein the locking tabs slideably engage interior walls of the tubular body inside the internal cavity when the second cap moves between the top and bottom ends.

20. The sample container according to claim 2, wherein the second cap has a castellated configuration.
